(19) United States
(12) Patent Application Publication
(10) Pub. No.: US 2024/0408195 A1
(43) Pub. Date: Dec. 12, 2024

(54) EPSTEIN-BARR VIRUS VACCINES

(71) Applicant: The USA, as represented by the Secretary, Dept, of Health and Human Services, Bethesda, MD (US)

(72) Inventors: Masaru KANEKIYO, North Bethesda, MD (US); Gary J. NABEL, Chestnut Hill, MA (US); Jeffrey COHEN, Silver Spring, MD (US); Wei BU, Potomac, MD (US)

(73) Assignee: The USA, as represented by the Secretary, Dept, of Health and Human Services, Bethesda, MD (US)

(21) Appl. No.: 18/659,639

(22) Filed: May 9, 2024

Related U.S. Application Data
(62) Division of application No. 16/922,322, filed on Jul. 7, 2020, now Pat. No. 12,005,115.
Application No. 16/922,322 is a division of application No. 15/028,655, filed on Apr. 11, 2016, now Pat. No. 10,744,199.
(60) Provisional application No. 61/921,284, filed on Dec. 27, 2013.
(60) Provisional application No. 61/889,840, filed on Oct. 11, 2013.

(51) Int. Cl. A61K 39/245; A61K 39/00; A61K 39/12; C07K 14/00; C07K 14/005; C07K 14/195; C07K 16/08; C12N 7/00

(52) U.S. Cl. CPC A61K 39/245; A61K 39/12; C07K 14/00; C07K 14/005; C07K 14/195; C07K 16/085; C12N 7/00; A61K 2039/55505; A61K 2039/575; A61K 2039/64; C07K 2317/33; C07K 2317/76; C07K 2319/00; C07K 2319/40; C07K 2319/735; C12N 2710/16222; C12N 2710/16234; C12N 2710/16271

(57) ABSTRACT

Vaccines are provided that elicit neutralizing antibodies to Epstein-Barr virus (EBV). Some vaccines comprise nanoparticles that display envelope proteins from EBV on their surface. The nanoparticles comprise fusion proteins comprising a monomeric subunit of a self-assembly protein, such as ferritin, joined to at least a portion of an EBV envelope protein. The fusion proteins self-assemble to form the envelope protein-displaying nanoparticles. Such vaccines can be used to vaccinate an individual against infection by different types of Epstein-Barr viruses as well as Epstein-Barr viruses that are antigenically divergent from the virus from which the EBV envelope protein was obtained. Also provided are fusion proteins and nucleic acid molecules encoding such proteins.

### CROSS REFERENCE TO RELATED APPLICATIONS

This application is a Division of U.S. application Ser. No. 16/922,322, filed Jul. 7, 2020, which is a divisional of U.S. patent application Ser. No. 15/028,655, filed Apr. 11, 2016, now U.S. Pat. No. 10,744,199, Issued on Aug. 18, 2020, which is a national stage application under 35 U.S.C. 371 and claims the benefit of PCT Application No. PCT/US2014/060142 having an international filing date of Oct. 10, 2014, which designated the United States, which PCT application claimed the benefit of U.S. Provisional Application No. 61/889,840 filed October 2013, and U.S. Provisional Application No. 61/921,284 filed Dec. 27, 2013, the disclosure of each of which are incorporated herein by reference.

### INCORPORATION BY REFERENCE OF SEQUENCE LISTING

The content of the electronically submitted sequence listing, file name: A298288_Substitute sequence listing as filed.xml; size: 385,800 bytes; and date of creation: Aug. 28, 2024, filed herewith, is incorporated herein by reference in its entirety.

### SUMMARY OF THE INVENTION

The present invention provides novel, nanoparticle-based vaccines for Epstein-Barr virus that are easily manufactured, potent, and which elicit neutralizing antibodies to Epstein-Barr virus. In particular, the present invention provides novel Epstein-Barr virus protein-ferritin nanoparticle (np) vaccines. Such nanoparticles comprise fusion proteins, each of which comprises a monomeric subunit of a self-assembly protein, such as ferritin, joined to an immunogenic portion of an Epstein-Barr virus envelope protein. Because such nanoparticles display Epstein-Barr virus proteins on their surface, they can be used to vaccinate an individual against Epstein-Barr virus.

One embodiment of the present invention is a nanoparticle that includes a first fusion protein that is joined to at least one immunogenic portion from a first Epstein-Barr virus envelope protein that is selected from the group consisting of gp350, gH, gL, gp42, gB and BMRF2. The first fusion protein includes at least 25 contiguous amino acids from a monomeric subunit protein capable of self-assembling into a nanoparticle. Further, the nanoparticle expresses the at least one immunogenic portion on its surface.

The monomeric subunit of the self-assembly protein can be selected from a monomeric ferritin subunit protein, a monomeric encapsulin protein, a monomeric 03-33 protein, a monomeric SOR protein, a monomeric LS protein and a monomeric PDC protein. In the embodiment of a monomeric ferritin subunit protein, it can be selected from a bacterial, plant, algal, insect, fungal, and mammalian ferritin. More specifically, the monomeric subunit protein can be selected from a monomeric subunit of a Helicobacter pylori ferritin protein, a monomeric subunit of a Escherichia coli protein and a monomeric subunit of a bullfrog ferritin protein. Also, the monomeric ferritin subunit protein can be a hybrid protein that includes at least a portion of a bullfrog ferritin protein joined to at least a portion of a ferritin protein that is selected from a Helicobacter pylori ferritin protein and Escherichia coli ferritin protein.

The monomeric subunit self-assembling protein can include at least 25 contiguous amino acids of, be at least about 80% identical to or comprise an amino acid sequence selected from SEQ ID NO:2, SEQ ID NO:5, SEQ ID NO: 8, SEQ ID NO: 11, SEQ ID NO: 14, SEQ ID NO: 17, SEQ ID NO:20, SEQ ID NO:23, SEQ ID NO:26 and SEQ ID NO:29.

The first Epstein-Barr virus envelope protein can be from Epstein-Barr virus type 1 or Epstein-Barr virus type 2. Also, the at least one immunogenic portion from the first Epstein-Barr virus envelope protein can include at least 100 amino acids from an amino acid sequence selected from SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO: 59, SEQ ID NO: 62, SEQ ID NO: 65, SEQ ID NO: 68, SEQ ID NO: 130, SEQ ID NO: 132, SEQ ID NO: 134 and SEQ ID NO: 136. Further, the at least one immunogenic portion can include at least one domain selected from EBV gp350 Domain I, EBV gp350) Domain II and EBV gp350 Domain III. In addition, the at least one immunogenic portion can include the amino acid sequences of EBV gp350 Domain I and Domain II. Further, the at least one immunogenic portion can include the EBV gp350 CR2-binding site.

In this embodiment, the first EBV envelope protein can include an amino acid sequence that is at least about 80% identical to, is identical to or can elicit an immune response to an amino acid sequence selected from SEQ ID NO:32, SEQ ID NO:35, SEQ ID NO: 38, SEQ ID NO:41, SEQ ID NO:44, SEQ ID NO:47, SEQ ID NO:50, SEQ ID NO:53, SEQ ID NO:56, SEQ ID NO:59, SEQ ID NO: 62, SEQ ID NO: 65, SEQ ID NO:68, SEQ ID NO: 130, SEQ ID NO: 132, SEQ ID NO: 134 and SEQ ID NO: 136.

The first fusion protein can comprise a linker sequence.

The nanoparticle can elicit an immune response against an Epstein-Barr virus, including a strain of Epstein-Barr virus that is heterologous to the strain Epstein-Barr virus from which the Epstein-Barr virus envelope protein was obtained, as well as to an Epstein-Barr virus that is antigenically divergent from the Epstein-Barr virus from which the Epstein-Barr virus envelope protein was obtained.

The first fusion protein can include an amino acid sequence that is at least about 80% identical to or is identical to a sequence selected from SEQ ID NO: 71, SEQ ID NO: 74, SEQ ID NO: 77, SEQ ID NO:80, SEQ ID NO:83, SEQ ID NO:86, SEQ ID NO:89, SEQ ID NO: 92, SEQ ID NO:95, SEQ ID NO:98, SEQ ID NO: 101, SEQ ID NO: 104, SEQ ID NO: 128, SEQ ID NO: 134, SEQ ID NO: 142, SEQ ID NO: 144 and SEQ ID NO: 146, wherein the nanoparticle elicits an immune response against an Epstein-Barr virus.

The nanoparticle can further include a second fusion protein that includes at least one immunogenic portion from a second Epstein-Barr virus envelope protein that is selected from gp350, gH, gL, gp42, gB and BMRF2. In this embodiment, the first and second Epstein-Barr virus envelope proteins are not the same and the nanoparticle expresses the at least one immunogenic portion from a second fusion protein on its surface.

A further embodiment of the present invention is a vaccine composition that includes any one of the foregoing described nanoparticles. The vaccine composition can further include at least one additional nanoparticle that includes a second fusion protein with at least 25 contiguous amino acids from a monomeric subunit protein that can self-assemble into a nanoparticle and that is joined to at least one immunogenic portion from a second Epstein-Barr envelope protein that can be gp350, gH, gL, gp42, gB and BMRF2. In this embodiment, the at least one immunogenic portion of the second fusion protein is from an Epstein-Barr virus envelope protein from a different strain of Epstein-Barr virus than the first Epstein-Barr virus envelope protein and the nanoparticle expresses the at least one immunogenic portion of the second fusion portion on its surface.

A further embodiment of the present invention is a method to produce a vaccine against Epstein-Barr virus. The method includes expressing a fusion protein that includes at least 25 contiguous amino acids from a monomeric subunit protein that can self-assemble into a nanoparticle and that is joined to at least one immunogenic portion from a first Epstein-Barr virus envelope protein selected from gp350, gH, gL, gp42, gB and BMRF2. The step of expressing is conducted under conditions such that the fusion protein forms a nanoparticle displaying the at least one immunogenic portion of an Epstein-Barr virus envelope protein on its surface. The method further includes recovering the nanoparticle.

A further embodiment of the present invention is a method to vaccinate an individual against Epstein-Barr virus that includes administering a nanoparticle to an individual such that the nanoparticle elicits an immune response against Epstein-Barr virus. The nanoparticle includes a fusion protein that includes at least 25 contiguous amino acids from a monomeric subunit protein that can self-assemble into a nanoparticle and that is joined to at least one immunogenic portion of a first Epstein-Barr virus protein selected from gp350, gH, gL, gp42, gB and BMRF2. In this embodiment, the nanoparticle displays the at least one immunogenic portion from an Epstein-Barr virus envelope protein on its surface. In this method to vaccinate, the nanoparticle can elicit an immune response to an Epstein-Barr virus strain that is heterologous to the strain of Epstein-Barr virus from which the envelope protein was obtained, or to an Epstein-Barr virus that is antigenically divergent from the Epstein-Barr virus from which the envelope protein was obtained.

The method to vaccinate can include administering to the individual a first vaccine composition and then at a later time administering a second vaccine composition that includes a nanoparticle that comprises a fusion protein comprising at least 25 contiguous amino acids from a monomeric subunit protein that can self-assemble into a nanoparticle and that is joined to at least one immunogenic portion of the first Epstein-Barr virus envelope protein selected from gp350, gH, gL, gp42, gB and BMRF2. In this embodiment, the nanoparticle displays the at least one immunogenic portion of an Epstein-Barr virus envelope protein on its surface. In this embodiment, the fusion protein can include an amino acid sequence that is at least about 80% identical to or is identical to a sequence that is selected from SEQ ID NO:71, SEQ ID NO:74, SEQ ID NO:77, SEQ ID NO:80, SEQ ID NO: 83, SEQ ID NO:86, SEQ ID NO:89, SEQ ID NO:92, SEQ ID NO:95, SEQ ID NO:98, SEQ ID NO:101 SEQ ID NO:104, SEQ ID NO:128, SEQ ID NO:134, SEQ ID NO:142, SEQ ID NO:144 and SEQ ID NO:146.

In this embodiment, the second vaccine composition may be administered between ten days and four weeks following administration of the first vaccine composition. In this embodiment, the second vaccine composition may be administered between ten days and two months following administration of the first vaccine composition. In one embodiment, a third vaccine composition may be administered six months after administration of the first vaccine composition.

A further embodiment of the present invention is a fusion protein that includes at least 25 contiguous amino acids from a monomeric subunit protein that can self-assemble into a nanoparticle and that is joined to at least one immunogenic portion from an Epstein-Barr virus envelope protein selected from gp350, gH, gL, gp42, gB and BMRF2. In this embodiment, the monomeric subunit can be selected from a monomeric ferritin subunit protein, a monomeric encapsulin protein, a monomeric 03-33 protein, a monomeric SOR protein, a monomeric LS protein and a monomeric PDC protein. The monomeric ferritin subunit protein can be selected from a bacterial, plant, algal, insect, fungal and mammalian ferritin. Further, the monomeric ferritin subunit protein can be selected from a monomeric subunit of a Helicobacter pylori ferritin protein, a monomeric subunit of an Escherichia coli ferritin protein and a monomeric subunit of a bullfrog ferritin protein. Further, the monomeric ferritin subunit protein can be a hybrid protein that includes at least a portion of a bullfrog ferritin protein joined to at least a portion of a Helicobacter pylori ferritin protein or an Escherichia coli ferritin protein. Still further, the monomeric ferritin subunit protein can comprise an amino acid sequence that is at least about 80% identical to or is identical to or comprises at least 25 contiguous amino acids selected from SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26 and SEQ ID NO:29, wherein the fusion protein can self-assemble into nanoparticles. The fusion protein can be an Epstein-Barr virus envelope protein type 1 or type 2.

The at least one immunogenic portion of the fusion protein can comprise at least 100 amino acids from an amino acid sequence selected from SEQ ID NO:32, SEQ ID NO:35. SEQ ID NO:38, SEQ ID NO:41, SEQ ID NO:44, SEQ ID NO:47, SEQ ID NO:50, SEQ ID NO: 53, SEQ ID NO:56, SEQ ID NO:59, SEQ ID NO:62, SEQ ID NO:65, SEQ ID NO:68. SEQ ID NO:130, SEQ ID NO:132 and SEQ ID NO:136. The at least one immunogenic portion can include at least one domain selected from EBV gp350 Domain I, EBV gp350 Domain II and EBV gp350 Domain III. Further, the at least one immunogenic portion can include the amino acid sequences of EBV gp350 Domain I and Domain II. Still further, the at least one immunogenic portion can include the EBV gp350 CR2-binding site.

The EBV envelope protein can include an amino acid sequence at least about 80% identical to or is identical to an amino acid sequence selected from SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:130, SEQ ID NO:132 and SEQ ID NO:136. The EBV envelope protein of the fusion protein can be capable of eliciting an immune response to the protein that includes an amino acid sequence selected from SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:130, SEQ ID NO:132 and SEQ ID NO:136.

The fusion protein can also include a linker sequence.

Further embodiments of the present invention include nucleic acid molecules that encode the foregoing described fusion proteins. The nucleic acid molecule can be functionally linked to a promoter.

A still further embodiment of the present invention is a recombinant cell that includes the foregoing nucleic acid molecule.

A further embodiment of the present invention is a recombinant virus that includes the foregoing described nucleic acid molecule.

### BACKGROUND

Epstein-Barr virus (EBV), also referred to as human herpesvirus 4 (HHV-4), is the principle etiological agent of infectious mononucleosis (IM) and is also associated with several human cancers, with more than 300,000 people being affected each year worldwide. The World Health Organization estimates that 95% of adults worldwide have been infected with EBV and are carriers of the virus. For the majority of individuals, EBV does not cause any symptoms and is indistinguishable from common, mild childhood illnesses. Currently, there is no vaccine for EBV. However, prevention of IM and EBV-associated malignancies through vaccination would have a substantial public health and economic benefit.

EBV has a linear, double-stranded DNA genome comprising approximately 192 kilobases (KB) of DNA, surrounded by a protein capsid. The capsid is surrounded by a protein tegument, which in turn is surrounded by an envelope. The EBV envelope contains several proteins, including glycoprotein gp350, gH, gB, gM, gp42, gL, gp78, gp150 and gN. The most abundant envelope glycoprotein is 350/220 (gp350), which binds complement receptor 2 (CR2 or CD21) enabling EBV infection of B cells, while glycoproteins gH and gp42 bind integrins and human leukocyte antigen class II molecules, respectively. Antibodies directed toward the putative CR2-binding site (CR2BS) on gp350 have been shown to potently inhibit EBV infections of B cells, and thus vaccine efforts against EBV have been largely focused on gp350.

In addition to infecting B cells, EBV also infects epithelial cells in the oropharynx where it is thought to spread to B cells. Current data suggests that infection of epithelial cells by EBV is initiated by attachment of EBV BMRF2 protein to epithelial cells followed by binding of EBV gH/gL to integrins, which serve as receptors for the virus on epithelial cells. Antibodies to gH/gL in human plasma blocks EBV infection of epithelial cells (Bu and Cohen, unpublished data) suggesting that a vaccine capable of inducing antibodies to EBV gH/gL may help to prevent infection or human disease due to EBV.

While work on EBV vaccines has continued, to date there is no efficacious EBV vaccine. For example, a recently completed phase 2 clinical trial of an adjuvanted recombinant gp350 protein vaccine showed that the vaccine did not protect against EBV infection but did reduce the incidence of IM by 78% (Sokal, E. M., et al. J Infect Dis. 196:1749-53,2007). Thus, there remains a need for an efficacious Epstein-Barr virus vaccine that provides robust protection against EBV. The present invention meets this need by providing a novel ENV-SA protein-based nanoparticle (ENV-SA np) vaccine that is easily manufactured, potent, and elicits neutralizing antibodies against EBV.

### BRIEF DESCRIPTION OF THE FIGURES

FIG. 1. Molecules essential for EBV infection of B cells and epithelial cells. Entry of EBV in B cells is initiated by attachment of EBV glycoprotein gp350 to its' cellular receptor CR2/CD21, followed by binding of viral glycoprotein gp42 (which also interacts with EBV gH/gL forming a trimer) to HLA class II. This triggers membrane fusion which is completed by glycoprotein gB. Entry of EBV into epithelial cells is thought to be initiated by binding of EBV BMRF2 to its cellular receptor integrins, followed by binding of gH/gL heterodimer to their cellular receptor integrins, and membrane fusion is subsequently triggered and completed by gB. (Figure from Longnecker R, Kieff E, and Cohen JI. Epstein-Barr virus. In: Fields Virology 2013).

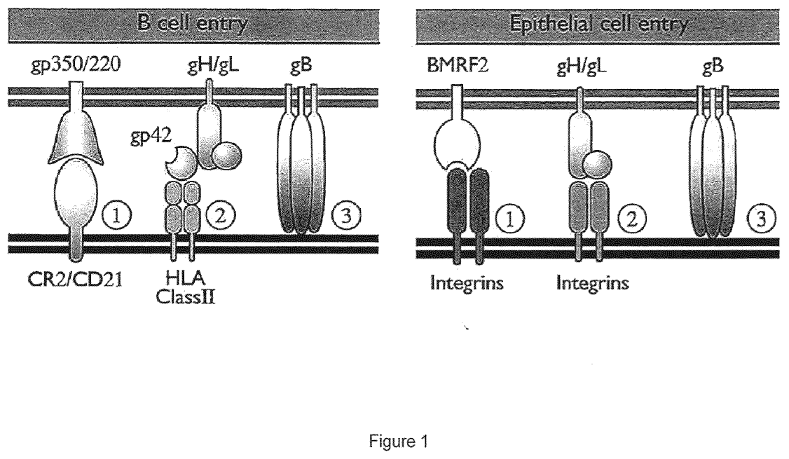

FIG. 2. Molecular design of truncated gp350 variants and gp350-based nanoparticles. (A) Molecular design of truncated gp350 containing CR2-binding site (CR2BS). Crystal structure of EBV gp350 receptor-binding domain (RBD) (PDB: 2h6o) is shown in surface representation (top). The N- and C-terminal residues in the crystal structures are indicated with amino acid number (B95-8 numbering) (left). Schematic representation of full length gp350 and its truncated variants (bottom). Gp350_{ecto} is truncated by removing the transmembrane (TM) and cytoplasmic tail (CT). Gp350_{RBD} is identical to the construct used for the crystallography study (Szakonyi, G., et al., Nat Struct Mol Biol. 13, 996-1001, 2006) and it contains intact domains I-III and an extended tail. Gp350_{DI/II/III} and gp350_{DI/II} are truncated further while retaining intact domains I-III and I-II, respectively. (B) Design of soluble monomeric gp350 and gp350-based nanoparticles. The soluble gp350 monomer constructs are made by genetically adding a modified bovine prolactin leader sequence (bPRL) and a poly histidine tag (HIS) at the N- and the C-terminus of gp350, respectively. The gp350-based ferritins are constructed by genetically fusing a bPRL sequence followed by gp350 variants to the N-terminus of ferritin (Helicobacter pylori-bullfrog hybrid (Hp) or Escherichia coli-bullfrog hybrid (Ec)) with a Ser-Gly linker between gp350 variants and ferritin. The gp350-based encapsulins are constructed by genetically fusing a human CD5 leader (hCD5) sequence and gp350 variants to the N- and the C-termini of encapsulin (Thermotoga maritima), respectively with a (Ser-Gly₃)₂ linker between gp350 variants and encapsulin. These fusion genes are then cloned into the mammalian expression vector (CMV8x/R). Soluble gp350_{ecto} and gp350_{DI/II/III} are designated as VRC 3796 and 3797, respectively. Gp350 variants fused with Ec ferritin, Hp ferritin and encapsulin are designated as VRC 3421-3424, 3425-3428 and 3429-3432. respectively.

FIG. 3. Molecular structure and design of gH/gL ferritin-based, gH/gL/gp42 ferritin-based and gH/gL/gp42 encapsulin-based nanoparticles. (A) Crystal structure of EBV gH/gL heterodimer (PDB: 3PHF). (B) Ribbon diagram of EBV gp42 (PDB: 3FD4). (C) Schematic representation of full-length gH, gH_{D1234}-ferritin fusion protein, gH _{D1234} soluble, full-length gL, full-length gp42, encapsulin-gp42 and gp42 soluble proteins, gH-ferritin fusion protein is generated by fusion of gH ectodomain (D₁₂₃₄) to the N-terminus of ferritin (Helicobacter pylori-bullfrog hybrid (Hp)). Soluble gH is constructed by deletion of transmembrane domain and cytoplasmic tail. gH _{D1234} is identical to the construct used for the crystallography study (Matsuura, H., et al. Proc. Natl. Acad. Sci. U.S.A. 107:22641-22646, 2010). EBV gL is the full-length wild type gL. The encapsulin-gp42 is constructed by fusing a human CD5 leader (hCD5) sequence to the N-termini of encapsulin (Thermotoga maritima), followed by (Ser-Gly₃)₂ linker, and followed by gp42 (with deletion of N-terminal amino acids 1-33). Soluble gp42 is constructed by fusing a human CD5 leader (hCD5)_sequence to the N-terminus truncated gp42 in place of gp42 amino 30 acids 1-33 which are deleted.

FIG. 4. Purification of truncated gp350 variants and gp350-based nanoparticles. (Left) Chromatograph resulting from size-exclusion chromatography (Superdex 200 10/300 GL) of immobilized metal ion (Ni⁺⁺) affinity chromatography purified, ferritin-based gp350) nanoparticles (gp350) ecto and gp350 DI/II/III) obtained from the supernatants of cells transfected with VRC 3796 and 3797 (Right) Chromatograph resulting from size-exclusion chromatography (Sephacryl S-500 16/60) of snowdrop lectin (Galanthus nivalis) affinity chromatography purified, encapsulin-based gp350 and ferritin-based gp350 nanoparticles obtained from the supernatants of cells transfected with VRC 3426 and 3430.

FIG. 5. Purification of gH/gL ferritin-based and gH/gL/gp42 ferritin-based nanoparticles. (A) Chromatograph resulting from size-exclusion chromatography (Sephacryl S-500 16/60 column) of snowdrop lectin (Galanthus nivalis) affinity chromatography purified, gH/gL and gH/gL/gp42 ferritin-based nanoparticles obtained from the supernatants of cells transfected with gH_{D1234}-ferritin and gL plasmids or gH_{D1234}-ferritin, gL, and soluble gp42 plasmids, respectively (B) Characterization of nanoparticles by SDS-PAGE. The bands corresponding to gH-ferritin, gp42, and gL are indicated. (C) Immunoprecipitation of gH/gL/gp42 encapsulin-based nanoparticle by mAbs. An anti-gp42 mAb (F2-1) and anti-gH/gL mAb (E1D1) were used to detect gH/gL/gp42 encapsulin-based nanoparticles. HC and LC denote antibody heavy and light chains, respectively.

FIG. 6. Electron microscopic (EM) analyses of gp350-based nanoparticles. Assembled ferritin and encapsulin nanoparticles are shown (left). The sites of fusion (N-termini on ferritin and C-termini on encapsulin) are shown as black dots (24 sites on a ferritin and 60 sites on a encapsulin). Negative stain transmission EM pictures of gp350-based nanoparticles (middle and right), using Hp ferritin (top) and encapsulin (bottom) platforms. Gp350-based nanoparticles are derived from VRC 3426, 3427, 3430 and 3431.

FIG. 7. Electron microscopic (EM) analysis of gH/gL ferritin and gH/gL/gp42 ferritin-based nanoparticles. Negative stain transmission EM images of gH/gL ferritin-based nanoparticles (left) and gH/gL/gp42 ferritin-based nanoparticles (right) are shown.

FIG. 8. Antigenic characterization of truncated gp350 variants and gp350-based nanoparticles. Binding properties of purified gp350 variants and gp350-based nanoparticles to anti-gp350 monoclonal antibodies (mAbs). Endpoint concentrations of binding mAbs were measured by ELISA (left). Immunoprecipitation of gp350-based nanoparticles by mAbs (right). A neutralizing anti-CR2BS mAb (72A1) and a non-neutralizing anti-gp350 (not specified, not anti-CR2BS) mAb (2L10) were used to detect gp350 variants and gp350-based nanoparticles. An anti-Influenza HA mAb (C179) was used as an isotype control. HC and LC denote antibody heavy and light chains, respectively. Soluble gp350 variants and gp350-based nanoparticles are derived from VRC 3796, 3797, 3426 and 3430.

FIG. 9. Comparison of immunogenicity of different gp350-variants and nanoparticle platforms. Immunogenicity of gp350 D₁₂₃-based nanoparticles (A) and gp350 RBD-based nanoparticles (B). BALB/c mice (n=5) were immunized intramuscularly with 5 μg of indicated gp350-based nanoparticles mixed with a Ribi adjuvant at weeks 0 and 3. Immune sera were collected 2 weeks after the first (1) and the second (2) immunization. Immune sera were analyzed by measuring antibody binding titer against soluble gp350_{ecto} by ELISA (left) and neutralizing titer (right). The endpoint titers of anti-gp350_{ecto} are shown (left). The neutralization assay was based on the Raji B cell line and a GFP reporter virus (Sashihara J., et al., Virology. 391, 249-256, 2009) and the titer is shown as a dilution of serum needed to inhibit viral entry by 50% (IC₅₀). Each dot represents individual mouse. Bar indicates mean and s.d. *, p<0.05: **, p<0.01: ***, p<0.001: ****, p<0.0001. Gp350-based nanoparticles used for immunization are derived from VRC 3422, 3423, 3426, 3427, 3430 and 3431.

FIG. 10. Comparison of immunogenicity of soluble gp350 variants and gp350-based nanoparticle. Immune sera were analyzed by measuring anti-gp350 antibody binding titer by ELISA (left) and LIPS (center) assay and neutralization IC₅₀ titer (right). LIPS assay was performed as previously described (Sashihara J., et al., Virology. 391, 249-256, 2009). Each dot represents individual mouse. Bar indicates mean and s.d. *, p<0.05: **, p<0.01: ***, p<0.001: ****, p<0.0001. Gp350 variants and gp350-based nanoparticles used for immunization are derived from VRC 3796, 3797, 3426 and 3430.

FIG. 11. Detection of CR2BS-directed antibodies in gp350-based nanoparticle-immune sera. (A) Surface plasmon resonance-based cross-competition assay of immune sera with an anti-CR2BS (72A1), an anti-gp350 (2L10, non-CR2BS directed) and an isotype control (C179, anti-influenza) mAbs. Each curve represents individual mouse. Cross-competition of immune sera by 72Al (top) and 2L10 (bottom) were shown by different immunization groups. (B) Relative percentages of CR2BS-directed and 2L10-like antibodies in the immune sera. Each dot represents individual mouse and bar indicates mean and s.d. *, p<0.05: **, p<0.01: ***, p<0.001: ****, p<0.0001. Gp350 ectodomain and gp350-based nanoparticles used for immunization are derived from VRC 3796, 3426 and 3430.

FIG. 12. Kinetics of serum neutralization titers after immunization with soluble gp350 and gp350 based nanoparticles. Groups of BALB/c mice (n=5) were immunized intramuscularly with 5 μg (upper panels) or 0.5 μg (lower panels) of soluble gp350 ectodomain (left), D₁₂₃-ferritin (center) or D₁₂₃-encapsulin (right) mixed with a Ribi adjuvant at weeks 0, 3, and 16. Immune sera were collected periodically after immunization and serum neutralization IC₅₀ titers were determined. gp350 ectodomain and gp350-based nanoparticles used for immunization are derived from VRC 3796, 3426 and 3430.

FIG. 13. Comparison of serum neutralization titers in EBV-positive human individuals and gp350-based nanoparticle-immunized mice. (left panel) Comparison of serum neutralization titer (IC₅₀) between a combination of EBV-seropositive individuals and persons with EBV-positive mononucleosis (n=18) and gp350-based nanoparticle-immunized mice (titers at 2 weeks after the second immunization, combined groups shown in FIGS. 9 and 10, n=35). (right panel) Comparison of serum neutralization titer (IC₅₀) between EBV-seropositive infectious mononucleosis human individuals (peak neutralization titers of each individual, n=15) and D₁₂₃-ferritin-immunized mice (titers at 2 weeks after the second immunization, combined groups shown in FIG. 6, n=10). Each dot represents individual serum sample and bar indicates mean and s.d. Gp350-based nanoparticles used for immunization are derived from VRC 3422, 3423, 3426, 3427, 3430 and 3431.

FIG. 14. Generation of bullfrog-E. coli and bullfrog-H. pylori hybrid ferritins. Crystal structure of ferritin subunit monomers of human (light chain), bullfrog (red cell lower subunit) and bacterial (E. coli non-heme ferritin, FtnA) are shown (left). The extended N-terminal parts (circled) in human and bullfrog ferritins are exposed on a surface of assembled ferritin nanoparticles and the corresponding region is missing in E. coli (and H. pylori) ferritin (circled with dashed line). To make hybrid ferritins the N-terminal extended part of bullfrog ferritin was transplanted to either E. coli or H. pylori ferritin. The plasmids encoding hybrid E. coli-bullfrog and H. pylori-bullfrog ferritins are designated as VRC 3384 and 3419, respectively.

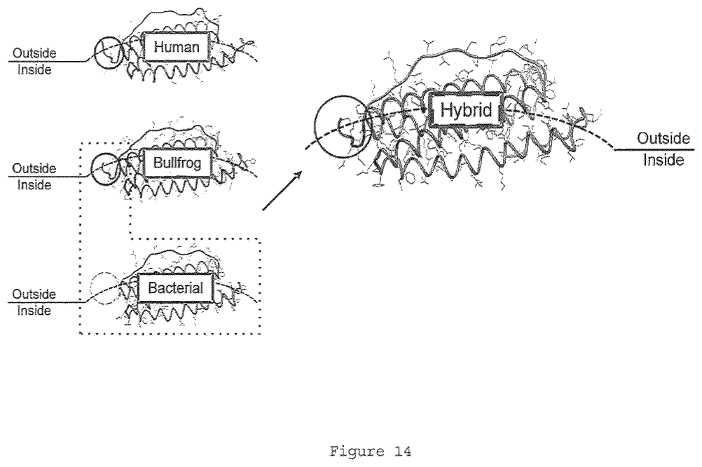

FIG. 15. Comparison of antibody titers of soluble gH/gL, soluble gH/gL/gp42, gH/gL ferritin-based nanoparticle and gH/gL/gp42 ferritin-based nanoparticles in immunized mouse sera. BALB/c mice (n=5) were immunized intramuscularly with 0.5 μg of the indicated proteins with a Ribi adjuvant at week 0 and 3. The antibody titers to gH/gL (A) and gp42 (B) in immune sera at week 5 were measured by a luciferase immunoprecipitation system (LIPS) assay. LIPS assay was preformed as previously described (Sashihara J., et al., Virology. 391, 249-256, 2009). Bar indicates the median with range. *, p<0.05: **, p<0.01: ***, p<0.001: ns, no significant difference.

FIG. 16. Comparison of neutralization of soluble gH/gL, soluble gH/gL/gp42, ferritin-based gH/gL nanoparticle and ferritin-based gH/gL/gp42 nanoparticles in B cells (A) and epithelial cells (B). Groups of BALB/c mice (n=5) were immunized with 0.5 μg indicated proteins with a Ribi adjuvant at week 0 and 3. (A) The neutralization assay was based on infection of B cells with GFP reporter virus (Sashihara J., et al., Virology. 391, 249-256, 2009) and the titer is shown as the dilution of serum capable to inhibit virus infection by 50% (IC₅₀). (B) The neutralization of EBV infection of epithelial cells was performed by incubation of mouse sera serially diluted in a 2-fold steps with GFP reporter virus for 2 hours. The mixture was added to SVK-CR2 cells (an epithelial cell line that expresses CR2, a receptor for EBV) in a 96-well plate and incubated for 3 days at 37° C. Cells were washed with 1× PBS, trypsinized, and fixed in 2% paraformaldehyde in PBS.GFP positive cells were quantified and the titer is shown as the dilution of serum able to inhibit virus infection by 50% (IC₅₀). Each dot represents an individual mouse. Bar indicates the median with range. *, p<0.05: **, p<0.01: ***, p<0.001, ns, no significant difference.

FIG. 17. (A) Immunization and sampling schedule. Comparison of kinetics of gH/gL antibody titers in sera from mice immunized with soluble gH/gL or gH/gL ferritin-based-nanoparticles (B) and soluble gH/gL/gp42 or gH/gL/gp42 ferritin-based-nanoparticles (C).

FIG. 18. (A) Immunization and sampling schedule. (B) Comparison of kinetics of gp42 antibody titers in sera from mice immunized with either soluble gH/gL/gp42 or gH/gL/gp42 ferritin-based-nanoparticles.

FIG. 19. (A) Immunization and sampling schedule. Comparison of kinetics of B cell neutralizing antibody titers in mice immunized with soluble gH/gL or gH/gL ferritin-based-nanoparticles (B) and soluble gH/gL/gp42 or gH/gL/gp42 ferritin-based-nanoparticles (C).

FIG. 20. (A) Immunization and sampling schedule. Comparison of kinetics of epithelial cell neutralizing antibody titers in mice immunized with soluble gH/gL or gH/gL ferritin-based-nanoparticles (B) and soluble gH/gL/gp42 or gH/gL/gp42 ferritin-based-nanoparticles (C).

FIG. 21. (A) B cell neutralizing antibody and (B) epithelial cell neutralizing antibody titers after the 3rd dose in sera of mice immunized with soluble proteins or ferritin-based-nanoparticles compared to sera from naturally infected humans. Each dot represents one individual. The median titers with the range are plotted. N. S. indicates that the difference was not statistically significant. **, p <0.01, ***, p <0.001, and *** p<0.0001.

FIG. 22. (A) Design of single polypeptides expressing either gH/gL-nanoparticles or gH/gL/gp42-nanoparticles. (B) SDS-PAGE analysis of purified gH/gL nanoparticles and gH/gL/gp42 nanoparticles by size exclusion chromatography (B). Lanes show proteins purified from cells co-transfected with plasmids expressing individual proteins (cotransfection) or one plasmid expressing a single polypeptide that is spontaneously cleaved inside the cell.

FIG. 23. Immunogenicity of EBV gp350-nanoparticles in cynomolgus monkeys. (A) Immunization schedule. (B) Titer of neutralizing virus in plasma from monkeys immunized with 50 μg of soluble gp350 ectodomain protein (left four bars), 25 μg of gp350 D123-ferritin (center four bars) or 25 μg of gp350 D123-encapsulin (right four bars) using the Sigma Adjuvant System.

FIG. 24. Survival curve for EBV gp350 immunized mice after challenge with recombinant vaccinia virus expressing EBV gp350. Mice were either not immunized (control) or immunized 3 times with 0.5 μg (left) or 5.0 μg (right) of gp350 ectodomain, gp350 D123-ferritin, or gp350 D123-encapsulin. Five mice were immunized in each group.

FIG. 25. Immunogenicity of EBV gp350-nanoparticles with no adjuvant, aluminum phosphate gel (alum), or Sigma Adjuvant System (SAS) adjuvant. Mice were immunized with 5 μg of gp350 D123-ferritin (left) or gp350 D123-encapsulin (right) at weeks 0, 4 and 16. Blood samples were collected 2 weeks after the final immunization and virus neutralization titers were measured.

### DETAILED DESCRIPTION OF THE INVENTION

The present invention relates to a novel vaccine for Epstein-Barr virus (EBV). More specifically, the present invention relates to novel fusion proteins comprising EBV envelope proteins, wherein the fusion proteins self-assemble into nanoparticles that display immunogenic portions of the EBV envelope proteins on their surface. Such nanoparticles are useful for vaccinating individuals against EBV. Accordingly, the present invention also relates to fusion proteins for producing such nanoparticles and nucleic acid molecules encoding such proteins. Additionally, the present invention relates to, methods of producing nanoparticles of the present invention, and methods of using such nanoparticles to vaccinate individuals against EBV.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the claims.

It must be noted that as used herein and in the appended claims, the singular forms “a,” “an,” and “the” include plural referents unless the context clearly dictates otherwise. For example, a nucleic acid molecule refers to one or more nucleic acid molecules. As such, the terms “a”, “an”, “one or more” and “at least one” can be used interchangeably. Similarly the terms “comprising”, “including” and “having” can be used interchangeably. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In addition to the above, unless specifically defined otherwise, the following terms and phrases, which are common to the various embodiments disclosed herein, are defined as follows:

As used herein, the term immunogenic refers to the ability of a specific protein, or a specific region thereof, to elicit an immune response to the specific protein, or to proteins comprising an amino acid sequence having a high degree of identity with the specific protein. According to the present invention, two proteins having a high degree of identity have amino acid sequences at least 80% identical, at least 85% identical, at least 87% identical, at least 90% identical, at least 92% identical, at least 94% identical, at least 96% identical, at least 98% identical or at least 99% identical.

As used herein, an immune response to a vaccine, or nanoparticle, of the present invention is the development in a subject of a humoral and/or a cellular immune response to an EBV envelope protein present in the vaccine. For purposes of the present invention, a “humoral immune response” refers to an immune response mediated by antibody molecules, including secretory (IgA) or IgG molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A cellular immune response also refers to the production of cytokines, chemokines and other such molecules produced by activated T-cells and/or other white blood cells, including those derived from CD4+ and CD8+ T-cells.

Thus, an immunological response may be one that stimulates CTLs, and/or the production or activation of helper T-cells. The production of chemokines and/or cytokines may also be stimulated. The vaccine may also elicit an antibody-mediated immune response. Hence, an immunological response may include one or more of the following effects: the production of antibodies (e.g., IgA or IgG) by B-cells; and/or the activation of suppressor, cytotoxic, or helper T-cells and/or T-cells directed specifically to a protein present in the vaccine. These responses may serve to neutralize infectivity, and/or mediate antibody-complement, or antibody dependent cell cytotoxicity (ADCC) to provide protection to an immunized individual. Such responses can be determined using standard immunoassays and neutralization assays, well known in the art.

According to the present invention all nomenclature used to describe EBV, and components thereof, is that commonly used by those skilled in the art. Thus, EBV (or HHV-4) refers to all Epstein-Barr viruses including, but not limited to, EBV Type I, EBV Type II, EBV strain B95-8, EBV strain Cao and EBV strain RAJI. A TYPE of EBV refers to either a TYPE I EBV or a TYPE II EBV. Methods of classifying Epstein-Barr viruses are known to those skilled in the art.

As used herein, neutralizing antibodies are antibodies that prevent EBV from infecting a cell, completing one round of replication or establishing latency. As defined herein, one round of replication refers the life cycle of the virus, starting with attachment of the virus to a host cell and ending with budding of newly formed virus from the host cell. This life cycle includes, but is not limited to, the steps of attaching to a cell, entering a cell, production of viral proteins, formation of new viral particles and budding of viral particles from the host cell membrane.

As used herein, broadly neutralizing antibodies are antibodies that neutralize more than one type and/or strain of EBV. For example, broadly neutralizing antibodies elicited against an envelope protein from a Type I EBV may neutralize a Type II virus.

As used herein, an EBV envelope protein refers to a full-length EBV envelope protein or any portion thereof, which is capable of eliciting an immune response. An epitope of a full-length EBV envelope protein refers to a portion of such protein that can elicit a neutralizing antibody response against the homologous EBV strain, i.e., a strain from which the EBV envelope protein is derived. In some embodiments, such an epitope can also elicit a neutralizing antibody response against a heterologous strain of EBV, i.e., a strain having an envelope protein that is not identical to the envelope protein of the immunogen.

As used herein, a variant refers to a protein, or nucleic acid molecule, the sequence of which is similar, but not identical to, a reference sequence, wherein the activity of the variant protein (or the protein encoded by the variant nucleic acid molecule) is not significantly altered. These variations in sequence can be naturally occurring variations or they can be engineered through the use of genetic engineering technique know to those skilled in the art. Examples of such techniques are found in Sambrook J, Fritsch E F. Maniatis T et al., in Molecular Cloning—A Laboratory Manual, 2nd Edition, Cold Spring Harbor Laboratory Press, 1989, pp. 9.31-9.57), or in Current Protocols in Molecular Biology, John Wiley & Sons, N.Y. (1989), 6.3.1-6.3.6, both of which are incorporated herein by reference in their entirety.

With regard to variants, any type of alteration in the amino acid, or nucleic acid, sequence is permissible so long as the resulting variant protein retains the ability to elicit neutralizing antibodies against an Epstein-Barr virus. Examples of such variations include, but are not limited to, deletions, insertions, substitutions and combinations thereof. For example, with regard to proteins, it is well understood by those skilled in the art that one or more (e.g., 2, 3, 4, 5, 6, 7, 8, 9 or 10), amino acids can often be removed from the amino and/or carboxy terminal ends of a protein without significantly affecting the activity of that protein. Similarly, one or more (e.g., 2, 3, 4, 5, 6, 7, 8, 9 or 10) amino acids can often be inserted into a protein without significantly affecting the activity of the protein.

As noted, variant proteins of the present invention can contain amino acid substitutions relative to the proteins disclosed herein. Any amino acid substitution is permissible so long as the activity of the protein is not significantly affected. In this regard, it is appreciated in the art that amino acids can be classified into groups based on their physical properties. Examples of such groups include, but are not limited to, charged amino acids, uncharged amino acids, polar uncharged amino acids, and hydrophobic amino acids. Preferred variants that contain substitutions are those in which an amino acid is substituted with an amino acid from the same group. Such substitutions are referred to as conservative substitutions.

Naturally occurring residues may be divided into classes based on common side chain properties:

1) hydrophobic: Met, Ala, Val, Leu, Ile;

2) neutral hydrophilic: Asn, Gln, Cys, Ser, Thr;

3) acidic: Asp, Glu;

4) basic: His, Lys, Arg;

5) residues that influence chain orientation: Gly, Pro; and

6) aromatic: Trp, Tyr, Phe.

For example, non-conservative substitutions may involve the exchange of a member of one of these classes for a member from another class.

In making amino acid changes, the hydropathic index of amino acids may be considered. Each amino acid has been assigned a hydropathic index on the basis of its hydrophobicity and charge characteristics. The hydropathic indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The importance of the hydropathic amino acid index in conferring interactive biological function on a protein is generally understood in the art (Kyte et al., 1982, J. Mol. Biol. 157:105-31). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those within ±1 are particularly preferred, and those within ±0.5 are even more particularly preferred.

It is also understood in the art that the substitution of like amino acids can be made effectively on the basis of hydrophilicity, particularly where the biologically functionally equivalent protein or peptide thereby created is intended for use in immunological invention, as in the present case. The greatest local average hydrophilicity of a protein, as governed by the hydrophilicity of its adjacent amino acids, correlates with its immunogenicity and antigenicity, i.e., with a biological property of the protein. The following hydrophilicity values have been assigned to these amino acid residues: arginine (+3.0); lysine (+3.0); aspartate (+3.0±1): glutamate (+3.0±1); serine (+0.3); asparagine (+0.2): glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, the substitution of amino acids whose hydrophilicity values are within ±2 is preferred, those within ±1 are particularly preferred, and those within ±0.5 are even more particularly preferred. One may also identify epitopes from primary amino acid sequences on the basis of hydrophilicity.

Desired amino acid substitutions (whether conservative or non-conservative) can be determined by those skilled in the art at the time such substitutions are desired. For example, amino acid substitutions can be used to identify important residues of the EBV envelope protein or the monomeric subunit protein, or to increase or decrease the immunogenicity, solubility or stability of the proteins described herein. Exemplary amino acid substitutions are shown below in Table 1.

TABLE 1
Amino Acid Substitutions

| Original | Exemplary |
|---|---|
| Amino Acid | Substitutions |
| Ala | Val, Leu, Ile |
| Arg | Lys, Gln, Asn |
| Asn | Gln |
| Asp | Glu |
| Cys | Ser, Ala |
| Gln | Asn |
| Glu | Asp |
| Gly | Pro, Ala |
| His | Asn, Gln, Lys, Arg |
| Ile | Leu, Val, Met, Ala |
| Leu | Ile, Val, Met, Ala |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

As used herein, the phrase significantly affect a proteins activity refers to a decrease in the activity of a protein by at least 10%, at least 20%, at least 30%, at least 40% or at least 50%. With regard to the present invention, such an activity may be measured, for example, as the ability of a protein to elicit neutralizing antibodies against an EBV virus or to self-assemble into a nanoparticle. Such activity may be measured by measuring the titer of such antibodies against EBV, or by measuring the types or strains of virus neutralized by the elicited antibodies. Methods of determining antibody titers and methods of performing virus neutralization assays are known to those skilled in the art. In addition to the activities described above, variant EBV proteins may also be analyzed for their ability to bind receptors (e.g., complement receptor 2) or other proteins. For example, it is understood that EBV gH binds EBV gL to form dimers. Likewise, it is understood that EBV gH binds EBV gL and gp42 to form trimers. Thus, variant EBV proteins may be analyzed for their ability to bind to one another. Methods of measuring such activities are known to those skilled in the art.

As used herein, a fusion protein is a recombinant protein containing amino acid sequence from at least two unrelated proteins that have been joined together, via a peptide bond, to make a single protein. The unrelated amino acid sequences can be joined directly to each other or they can be joined using a linker sequence. As used herein, proteins are unrelated, if their amino acid sequences are not normally found joined together via a peptide bond in their natural environment(s) (e.g., inside a cell). For example, the amino acid sequences of monomeric subunits that make up ferritin, and the amino acid sequences of ENV envelope proteins are not normally found joined together via a peptide bond.

The terms individual, subject, and patient are well-recognized in the art, and are herein used interchangeably to refer to any human or non-human primate susceptible to infection with EBV. Examples include, but are not limited to, humans and other primates, including non-human primates such as chimpanzees and other apes and monkey species. The terms individual, subject, and patient by themselves, do not denote a particular age, sex, race, and the like. Thus, individuals of any age, whether male or female, are intended to be covered by the present disclosure and include, but are not limited to the elderly, adults, children, babies, infants, and toddlers. Likewise, the methods of the present invention can be applied to any race, including, for example, Caucasian (white), African-American (black), Native American, Native Hawaiian, Hispanic, Latino, Asian, and European. An infected subject is a subject that is known to have EBV in their body.

As used herein, the terms exposed, exposure, and the like, indicate the subject has come in contact with a person of animal that is known to be infected with EBV.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

According to the present invention, vaccines are provided that elicit a neutralizing immune response against Epstein-Barr virus envelope proteins. Some vaccines disclosed herein may elicit an immune response against the entire envelope protein, while others may elicit an immune response against a specific region or portion of an envelope protein. Moreover, the inventors have discovered that specific fusion proteins comprising portions of envelope protein are useful for eliciting an immune response against Epstein-Barr viruses. Each of these embodiments will now be disclosed in detail below.

The inventors have discovered that fusion of an EBV envelope (ENV) protein with a self-assembly (SA) protein, to produce an ENV-SA fusion protein, results in a vaccine that elicits a robust immune response to EBV virus. Such ENV-SA fusion proteins self-assemble into nanoparticles that display immunogenic portions of the EBV protein on their surface. These nanoparticles are useful for vaccinating individuals against EBV. Thus, one embodiment of the present invention is an ENV-SA fusion protein comprising a self-assembling subunit protein disclosed herein joined to an EBV envelope (ENV) protein disclosed herein, wherein the ENV-SA fusion protein is capable of self-assembling into nanoparticles.

According to the present invention, a self-assembling (SA) subunit protein of the present invention is a full length, monomeric polypeptide, or any portion thereof, which is capable of directing self-assembly of monomeric self-assembling subunit proteins into a nanoparticle. Examples of self-assembly proteins of the present invention include ferritin, encapsulin, sulfur oxygenase reductase (SOR), lumazine synthase (LS) and pyruvate dehydrogenase complex (PDC) dihydrolipoamide acetyltransferase (E2). Representative examples of such proteins are listed below in Table 2.

TABLE 2

| SEQ | | |
|---|---|---|
| ID | | |
| NO | Organism | Comments |
| | | FERRITIN |
| 1 | Helicobacter pylori | Coding sequence for ferritin monomeric subunit protein from H. pylori |
| 2 | Helicobacter pylori | Amino acid sequence encoded by SEQ ID NO: 1 |
| 3 | Helicobacter pylori | Complement of SEQ ID NO1 |
| 4 | Escherichia coli | Coding sequence for ferritin monomeric subunit protein from E. coli |
| | | (gi 446839951_WP_000917207.1) |
| 5 | Escherichia coli | Amino acid sequence encoded by SEQ ID NO: 4 |
| 6 | Escherichia coli | Complement of SEQ ID NO4 |
| 7 | Rana catesbeiana | Coding sequence for bullfrog ferritin monomeric subunit |
| | | protein (gi 13675 gb AAA49524.1) |
| 8 | Rana catesbeiana | Amino acid sequence encoded by SEQ ID NO: 7 |
| 9 | Rana catesbeiana | Complement of SEQ ID NO: 7 |
| | | FERRITIN PROTEINS |
| 10 | Artificial Sequence | Coding sequence for H. pylori-ferritin/bullfrog-ferritin fusion protein |
| 11 | Artificial Sequence | Amino acid sequence encoded by SEQ ID NO: 10 |
| 12 | Artificial Sequence | Complement of SEQ ID NO10 |
| 13 | Artificial Sequence | Coding sequence for E. coli-ferritin/bullfrog-ferritin fusion protein |
| 14 | Artificial Sequence | Amino acid sequence encoded by SEQ ID NO: 13 |
| 15 | Artificial Sequence | Complement of SEQ ID NO: 13 |
| | | OTHER SELF-ASSEMBING MONOMERIC SUBUNITS |
| 16 | Thermotoga maritime | Coding sequence for encapsulin protein |
| 17 | Thermotoga maritime | Amino acid sequence encoded by SEQ ID NO: 16 |
| 18 | Thermotoga maritime | Complement of SEQ ID NO: 16 |
| 19 | Artificial Sequence | Coding sequence for Salmonella enteritis 03-33 protein |
| | | (gi 390136278 pdb 3VCD) |
| 20 | Artificial Sequence | Amino acid sequence encoded by SEQ ID NO: 19 |
| 21 | Artificial Sequence | Complement of SEQ ID NO: 19 |
| 22 | Acidianus ambivalens | Coding sequence for sulfur oxygenase reductase protein from |
| | | Acidianus ambivalens (gi 93279016 pdb 2CB2) |
| 23 | Acidianus ambivalens | Amino acid sequence encoded by SEQ ID NO: 22 |
| 24 | Acidianus ambivalens | Complement of SEQ ID NO: 22 |
| 25 | Aquifex aeolicus | Coding sequence for lumazine synthase protein from Aquifex aeolicus |
| | | (gi 18159011 pdb1HQK) |
| 26 | Aquifex aeolicus | Amino acid sequence encoded by SEQ ID NO: 25 |
| 27 | Aquifex aeolicus | Complement of SEQ ID NO: 25 |
| 28 | Bacillus | Coding sequence for dihydrolipoamide acetyltransferase (E2p) protein |
| | stearothermophilus | from Bacillus stearothermophilus (gi 4558102 pdb1B5S |
| 29 | Bacillus | Amino acid sequence encoded by SEQ ID NO: 28 |
| | stearothermophilus | |
| 30 | Bacillus | Complement of SEQ ID NO: 28 |
| | stearothermophilus | |
| | | EBV PROTEINS |
| 31 | Epstein Barr Virus | Coding sequence for EBV gp350 protein |
| 32 | Epstein Barr Virus | Amino acid sequence encoded by SEQ ID NO: 31 |
| 33 | Epstein Barr Virus | Complement of SEQ ID NO: 31 |
| 34 | Epstein Barr Virus | Coding sequence for EBV gp350 protein (2-860) ecto domain |
| 35 | Epstein Barr Virus | Amino acid sequence encoded by SEQ ID NO: 34 |
| 36 | Epstein Barr Virus | Complement of SEQ ID NO: 34 |
| 37 | Epstein Barr Virus | Coding sequence for EBV gp350 protein (2-470) RBD domain |
| 38 | Epstein Barr Virus | Amino acid sequence encoded by SEQ ID NO: 37 |
| 39 | Epstein Barr Virus | Complement of SEQ ID NO: 37 |
| 40 | Epstein Barr Virus | Coding sequence for EBV gp350 protein (4-153) Domain I |
| 41 | Epstein Barr Virus | Amino acid sequence encoded by SEQID NO: 40 |
| 42 | Epstein Barr Virus | Complement of SEQ ID NO: 40 |
| 43 | Epstein Barr Virus | Coding sequence for EBV gp350 protein (165-305) Domain II |
| 44 | Epstein Barr Virus | Amino acid sequence encoded by SEQ ID NO: 43 |
| 45 | Epstein Barr Virus | Complement of SEQ ID NO: 43 |
| 46 | Epstein Barr Virus | Coding sequence for EBV gp350 protein (317-426) Domain III |
| 47 | Epstein Barr Virus | Amino acid sequence encoded by SEQ ID NO: 46 |
| 48 | Epstein Barr Virus | Complement of SEQ ID NO: 46 |
| 49 | Epstein Barr Virus | Coding sequence for EBV gp350 protein (2-317) Domains I/II |
| 50 | Epstein Barr Virus | Amino acid sequence encoded by SEQ ID NO: 49 |
| 51 | Epstein Barr Virus | Complement of SEQ ID NO: 49 |
| 52 | Epstein Barr Virus | Coding sequence for EBV gp350 protein (2-425) Domains I/II/III |
| 53 | Epstein Barr Virus | Amino acid sequence encoded by SEQ ID NO: 52 |
| 54 | Epstein Barr Virus | Complement of SEQ ID NO: 52 |
| 55 | Epstein Barr Virus | Coding sequence for soluble EBV gp350 ectodomain protein |
| 56 | Epstein Barr Virus | Amino acid sequence encoded by SEQ ID NO: 55 |
| 57 | Epstein Barr Virus | Compliment of SEQ ID NO: 55 |
| 58 | Epstein Barr Virus | Coding sequence for soluble EBV gp350 Domains I/II/III |
| 59 | Epstein Barr Virus | Amino acid sequence encoded by SEQ ID NO: 58 |
| 60 | Epstein Barr Virus | Complement of SEQ ID NO: 58 |
| 61 | Epstein Barr Virus | Coding sequence for EBV gH protein |
| 62 | Epstein Barr Virus | Amino acid sequence encoded by SEQ ID NO: 61 |
| 63 | Epstein Barr Virus | Complement of SEQ ID NO: 61 |
| 64 | Epstein Barr Virus | Coding sequence for EBV gL protein |
| 65 | Epstein Barr Virus | Amino acid sequence encoded by SEQ ID NO: 64 |
| 66 | Epstein Barr Virus | Complement of SEQ ID NO: 64 |
| 67 | Epstein Barr Virus | Coding sequence for EBV gp42 protein |
| 68 | Epstein Barr Virus | Amino acid sequence encoded by SEQ ID NO: 67 |
| 69 | Epstein Barr Virus | Complement of SEQ ID NO: 67 |
| | | EBV FUSION PROTEINS |
| 70 | Artificial Sequence | Nucleic acid sequence encoding EBV gp350 (2-860) ecto-ferritin |
| | | (E. coli ferritin/bullfrog ferritin) fusion protein |
| 71 | Artificial Sequence | Amino acid sequence encoded by SEQ ID NO: 70 |
| 72 | Artificial Sequence | Complement of SEQ ID NO: 70 |
| 73 | Artificial Sequence | Nucleic acid sequence encoding EBV gp350 (2-470) RBD-ferritin |
| | | (E. coli ferritin/bullfrog ferritin) fusion protein |
| 74 | Artificial Sequence | Amino acid sequence encoded by SEQ ID NO: 73 |
| 75 | Artificial Sequence | Complement of SEQ ID NO: 73 |
| 76 | Artificial Sequence | Nucleic acid sequence encoding EBV gp350 (2-317) Domain |
| | | I/II-ferritin (E. coli ferritin/bullfrog ferritin) fusion protein |
| 77 | Artificial Sequence | Amino acid sequence encoded by SEQ ID NO: 76 |
| 78 | Artificial Sequence | Complement of SEQ ID NO: 76 |
| 79 | Artificial Sequence | Nucleic acid sequence encoding EBV gp350 (2-425) Domain |
| | | I/II/III-ferritin (E. coli ferritin/bullfrog ferritin) fusion protein |
| 80 | Artificial Sequence | Amino acid sequence encoded by SEQ ID NO: 79 |
| 81 | Artificial Sequence | Complement of SEQ ID NO: 79 |
| 82 | Artificial Sequence | Nucleic acid sequence encoding EBV gp350 (2-860) ecto-ferritin |
| | | (H. pylori ferritin/bullfrog ferritin) fusion protein |
| 83 | Artificial Sequence | Amino acid sequence encoded by SEQ ID NO: 82 |
| 84 | Artificial Sequence | Complement of SEQ ID NO: 82 |
| 85 | Artificial Sequence | Nucleic acid sequence encoding EBV gp350 (2-317) Domain |
| | | VII-ferritin (H. pylori ferritin/bullfrog ferritin) fusion protein |
| 86 | Artificial Sequence | Amino acid sequence encoded by SEQ ID NO: 85 |
| 87 | Artificial Sequence | Complement of SEQ ID NO: 85 |
| 88 | Artificial Sequence | Nucleic acid sequence encoding EBV gp350 (2-425) Domain |
| | | I/II/III-ferritin (H. pylori ferritin/bullfrog ferritin) fusion protein |
| 89 | Artificial Sequence | Amino acid sequence encoded by SEQ ID NO: 88 |
| 90 | Artificial Sequence | Complement of SEQ ID NO: 88 |
| 91 | Artificial Sequence | Nucleic acid sequence encoding EBV gp350 (2-470) RBD- |
| | | ferritin (H. pylori ferritin/bullfrog ferritin) fusion protein |
| 92 | Artificial Sequence | Amino acid sequence encoded by SEQ ID NO: 91 |
| 93 | Artificial Sequence | Complement of SEQ ID NO: 91 |
| 94 | Artificial Sequence | Nucleic acid sequence encoding EBV gp350 (2-860) ecto- |
| | | encapsulin fusion protein |
| 95 | Artificial Sequence | Amino acid sequence encoded by SEQ ID NO: 94 |
| 96 | Artificial Sequence | Complement of SEQ ID NO: 94 |
| 97 | Artificial Sequence | Nucleic acid sequence encoding EBV gp350 (2-470) RBD- |
| | | encapsulin fusion protein |
| 98 | Artificial Sequence | Amino acid sequence encoded by SEQ ID NO: 97 |
| 99 | Artificial Sequence | Complement of SEQ ID NO: 97 |
| 100 | Artificial Sequence | Nucleic acid sequence encoding EBV gp350 (317) Domain I/II- |
| | | encapsulin fusion protein |
| 101 | Artificial Sequence | Amino acid sequence encoded by SEQ ID NO: 100 |
| 102 | Artificial Sequence | Complement of SEQ ID NO: 100 |
| 103 | Artificial Sequence | Nucleic acid sequence encoding EBV gp350 (2-425) Domain |
| | | I/II/III-encapsulin fusion protein |
| 104 | Artificial Sequence | Amino acid sequence encoded by SEQ ID NO: 103 |
| 105 | Artificial Sequence | Complement of SEQ ID NO: 103 |
| 106 | Artificial Sequence | Nucleic acid sequence of VRC 3421 |
| 107 | Artificial Sequence | Nucleic acid sequence of VRC 3422 |
| 108 | Artificial Sequence | Nucleic acid sequence of VRC 3423 |
| 109 | Artificial Sequence | Nucleic acid sequence of VRC 3424 |
| 110 | Artificial Sequence | Nucleic acid sequence of VRC 3425 |
| 111 | Artificial Sequence | Nucleic acid sequence of VRC 3426 |
| 112 | Artificial Sequence | Nucleic acid sequence of VRC 3427 |
| 113 | Artificial Sequence | Nucleic acid sequence of VRC 3428 |
| 114 | Artificial Sequence | Nucleic acid sequence of VRC 3429 |
| 115 | Artificial Sequence | Nucleic acid sequence of VRC 3430 |
| 116 | Artificial Sequence | Nucleic acid sequence of VRC 3431 |
| 117 | Artificial Sequence | Nucleic acid sequence of VRC 3432 |
| 118 | Artificial Sequence | Nucleic acid sequence of VRC 3384 |
| 119 | Artificial Sequence | Nucleic acid sequence of VRC 3419 |
| 120 | Artificial Sequence | Nucleic acid sequence of VRC 3420 |
| 121 | Artificial Sequence | Nucleic acid sequence of VRC 3361 |
| 122 | Artificial Sequence | Nucleic acid sequence of VRC 3796 |
| 123 | Artificial Sequence | Nucleic acid sequence of VRC 3797 |
| 124 | Artificial Sequence | Nucleic acid sequence of VRC 2194 |
| 125 | Artificial Sequence | Nucleic acid sequence of VRC 2195 |
| 126 | Artificial Sequence | Nucleic acid sequence of VRC 2196 |
| 127 | Artificial Sequence | Nucleic acid sequence encoding gH-ferritin protein |
| 128 | Artificial Sequence | Protein encoded by SEQ ID NO: 127 |
| 129 | Artificial Sequence | Nucleic acid sequence encoding soluble gp42 protein |
| 130 | Artificial Sequence | Protein encoded by SEQ ID NO: 129 |
| 131 | Epstein Barr Virus | Nucleic acid sequence encoding gL protein |
| 132 | Epstein Barr Virus | Protein encoded by SEQ ID NO: 131 |
| 133 | Artificial Sequence | Nucleic acid sequence encoding encapsulin-gp42 |
| 134 | Artificial Sequence | Protein encoded by SEQ ID NO: 133 |
| 135 | Artificial Sequence | Nucleic acid sequence encoding soluble gH |
| 136 | Artificial Sequence | Protein encoded by SEQ ID NO: 135 |
| 137 | Picornvirus | Nucleic acid molecule encoding picornavirus 2A protease cleavage site |
| 138 | Picornavirus | Peptide encoded by SEQ ID NO: 137 (picornavirus 2A protease |
| | | cleavage site) |
| 139 | Human | Nucleic acid sequence encoding human CD5 leader peptide sequence |
| 140 | Human | Amino acid sequence encoded by SEQ ID NO: 139 (human CD5 |
| | | leader peptide sequence) |
| 141 | Artificial Sequence | Nucleic acid sequence encoding ferritin-gH-F2A-gL polyprotein |
| 142 | Artificial Sequence | Amino acid sequence encoded by SEQ ID NO: 141 (ferritin-gH- |
| | | F2A-gL polyprotein) |
| 143 | Artificial Sequence | Nucleic acid sequence encoding ferritin-gH-F2A-gL-F2A-gp42 |
| | | polyprotein |
| 144 | Artificial Sequence | Amino acid sequence encoded by SEQ ID NO: 143 (ferritin-gH- |
| | | F2A-gL-F2A-gp42 polyprotein) |
| 145 | Artificial Sequence | Nucleic acid sequence encoding ferritin-gH fusion protein having |
| | | SGGG linker |
| 146 | Artificial Sequence | Amino acid sequence encoded by SEQ ID NO: 145 (ferritin-gH |
| | | fusion protein having SGGG linker) |

Thus one embodiment of the present invention is an ENV-SA fusion protein comprising a self-assembling subunit protein selected from the group consisting of ferritin, encapsulin, sulfur oxygenase reductase, lumazine synthase and dihydrolipoamide acetyltransferase (E2), joined to an EBV envelope (ENV) protein disclosed herein, wherein the ENV-SA fusion protein is capable of self-assembling into nanoparticles.

In one embodiment, the self-assembly protein is ferritin. Ferritin forms a spherical protein found in all animals, bacteria, and plants, that acts primarily to control the rate and location of polynuclear Fe(III)₂O₃ formation through the transportation of hydrated iron ions and protons to and from a mineralized core. The spherical form of ferritin is made up of monomeric subunit proteins (also referred to as monomeric ferritin subunits), which are polypeptides having a molecule weight of approximately 17-20 kDa. An example of the sequence of one such monomeric ferritin subunit is represented by SEQ ID NO:2. Each monomeric ferritin subunit has the topology of a helix bundle which includes a four antiparallel helix motif, with a fifth shorter helix (the c-terminal helix) lying roughly perpendicular to the long axis of the 4 helix bundle. According to convention, the helices are labeled ‘A, B, C, and D & E’ from the N-terminus respectively. The N-terminal sequence lies adjacent to the capsid three-fold axis and extends to the surface, while the E helices pack together at the four-fold axis with the C-terminus extending into the particle core. The consequence of this packing creates two pores on the capsid surface. It is expected that one or both of these pores represent the point by which the hydrated iron diffuses into and out of the capsid. Following production, these monomeric ferritin subunit proteins self-assemble into the spherical ferritin protein. Thus, the spherical form of ferritin comprises 24 monomeric, ferritin subunit proteins, and has a capsid-like structure having 432 symmetry.

According to the present invention, a monomeric ferritin subunit of the present invention is a full length, single polypeptide of a ferritin protein, or any portion thereof, which is capable of directing self-assembly of monomeric ferritin subunits into the spherical form of the protein. Amino acid sequences from monomeric ferritin subunits of any known ferritin protein can be used to produce fusion proteins of the present invention, so long as the monomeric ferritin subunit is capable of directing self-assembly of the fusion protein into a nanoparticle displaying an EBV ENV protein on its surface. In one embodiment, the monomeric subunit is from a ferritin protein selected from the group consisting of a bacterial ferritin protein, a plant ferritin protein, an algal ferritin protein, an insect ferritin protein, a fungal ferritin protein and a mammalian ferritin protein. In one embodiment, the ferritin protein is from Helicobacter pylori. In one embodiment, the ferritin protein is from E. coli. In one embodiment, the ferritin protein is bullfrog ferritin. In one embodiment, the ferritin protein comprises amino acid sequences from one or more ferritin proteins selected from the group consisting of H. pylori ferritin, E. coli ferritin and bullfrog ferritin. Amino acid sequences from representative ferritin proteins of the present invention are disclosed herein as SEQ ID NO:2 (H. pylori ferritin), SEQ ID NO:5 (E. coli ferritin), SEQ ID NO:8 (bullfrog ferritin), SEQ ID NO:11 (H. pylori ferritin-bullfrog ferritin fusion) and SEQ ID NO:14 (E. coli ferritin-bullfrog ferritin fusion).

In one embodiment, the self-assembly protein is encapsulin. According to the present invention, a monomeric encapsulin subunit of the present invention is a full length, single polypeptide of an encapsulin protein, or any portion thereof, which is capable of directing self-assembly of monomeric encapsulin subunits into a nanoparticle. Amino acid sequences from monomeric encapsulin subunits of any known encapsulin protein can be used to produce fusion proteins of the present invention, so long as the monomeric encapsulin subunit is capable of directing self-assembly of the fusion protein into a nanoparticle displaying an EBV ENV protein on its surface. The amino acid sequence of a representative encapsulin protein is disclosed herein as SEQ ID NO:17.

In one embodiment, the self-assembly protein is artificially designed Salmonella enteritis 03-33 subunit protein. According to the present invention, a monomeric 03-33 subunit of the present invention is a full length, single polypeptide of an 03-33 protein, or any portion thereof, which is capable of directing self-assembly of monomeric 03-33 subunits into a nanoparticle. Amino acid sequences from monomeric 03-33 subunits of any known 03-33 protein can be used to produce fusion proteins of the present invention, so long as the monomeric 03-33 subunit is capable of directing self-assembly of the fusion protein into a nanoparticle displaying an EBV ENV protein on its surface. The amino acid sequence of a representative 03-33 protein is disclosed herein as SEQ ID NO:20.

In one embodiment, the self-assembly protein is sulfur oxygenase reductase (SOR). According to the present invention, a monomeric SOR subunit of the present invention is a full length, single polypeptide of an SOR protein, or any portion thereof, which is capable of directing self-assembly of monomeric SOR subunits into a nanoparticle. Amino acid sequences from monomeric SOR subunits of any known SOR protein can be used to produce fusion proteins of the present invention, so long as the monomeric SOR subunit is capable of directing self-assembly of the fusion protein into a nanoparticle displaying an EBV ENV protein on its surface. The amino acid sequence of a representative SOR protein is disclosed herein as SEQ ID NO:23.

In one embodiment, the self-assembly protein is lumazine synthase (LS). According to the present invention, a monomeric LS subunit of the present invention is a full length, single polypeptide of an LS protein, or any portion thereof, which is capable of directing self-assembly of monomeric LS subunits into a nanoparticle. Amino acid sequences from monomeric LS subunits of any known LS protein can be used to produce fusion proteins of the present invention, so long as the monomeric LS subunit is capable of directing self-assembly of the fusion protein into a nanoparticle displaying an EBV ENV protein on its surface. The amino acid sequence of a representative LS protein is disclosed herein as SEQ ID NO: 26.

In one embodiment, the self-assembly protein is pyruvate dehydrogenase complex (PDC) dihydrolipoamide acetyltransferase (E2p). According to the present invention, a monomeric E2p subunit of the present invention is a full length, single polypeptide of an E2p protein, or any portion thereof, which is capable of directing self-assembly of monomeric E2p subunits into a nanoparticle. Amino acid sequences from monomeric E2p subunits of any known E2p protein can be used to produce fusion proteins of the present invention, so long as the monomeric E2p subunit is capable of directing self-assembly of the fusion protein into a nanoparticle displaying an EBV ENV protein on its surface. The amino acid sequence of a representative E2p protein is disclosed herein as SEQ ID NO:29.

ENV-SA fusion proteins of the present invention need not comprise the full-length sequence of a monomeric subunit polypeptide of a self-assembly (SA) protein. Portions, or regions, of the monomeric SA subunit protein can be utilized so long as the portion comprises an amino acid sequence that directs self-assembly of the EBV-SA fusion protein into a nanoparticle. One example of such a portion is located between amino acids 5 and 167 of the Helicobacter pylori ferritin protein. More specific regions of the ferritin protein are described in Zhang, Y. Self-Assembly in the Ferritin Nano-Cage Protein Super Family. 2011, Int. J. Mol. Sci., 12, 5406-5421, which is incorporated herein by reference in its entirety. One embodiment of the present invention is an ENV-SA fusion protein comprising an Epstein-Barr virus ENV protein of the present invention joined to at least 25 contiguous amino acids, at least 50 contiguous amino acids, at least 75 contiguous amino acids, at least 100 contiguous amino acids, or at least 150 contiguous amino acids from a protein selected from the group consisting of ferritin, encapsulin, sulfur oxygenase reductase, lumazine synthase and pyruvate dehydrogenase complex (PDC) dihydrolipoamide acetyltransferase (E2), wherein the ENV-SA fusion protein is capable of self-assembling into nanoparticles. One embodiment of the present invention is an ENV-SA fusion protein comprising an ENV protein of the present invention joined to at least 25 contiguous amino acids, at least 50contiguous amino acids, at least 75 contiguous amino acids, at least 100 contiguous amino acids, or at least 150 contiguous amino acids from an amino acid sequence selected from the group consisting of SEQ ID NO:2, SEQ ID NO:5 SEQ ID NO:8, SEQ ID NO:11, SEQ ID NO:14, SEQ ID NO:17, SEQ ID NO:20, SEQ ID NO:23, SEQ ID NO:26 and SEQ ID NO:29, wherein the ENV-SA fusion protein is capable of self-assembling into nanoparticles. In one embodiment of the present, the ENV-SA fusion protein comprises an ENV-protein of the present invention joined to at least 25 contiguous amino acids, at least 50 contiguous amino acids, at least 75 contiguous amino acids, at least 100 contiguous amino acids, or at least 150 contiguous amino acids from amino acid residues 5-167 of SEQ ID NO: 2, SEQ ID NO:5 or SEQ ID NO:8, wherein the ENV-SA fusion protein is capable of self-assembling into nanoparticles.

As has been previously discussed, it is well-known in the art that some variations can be made in the amino acid sequence of a protein without affecting the activity of that protein. Such variations include insertion of amino acid residues, deletions of amino acid residues, and substitutions of amino acid residues. Thus, in one embodiment, the sequence of a SA protein subunit is divergent enough from the sequence of a SA protein subunit found in nature, such that when the variant SA protein subunit is introduced into an animal, such as a mouse, it does not result in the production of antibodies that react with the natural SA protein. According to the present invention, such a monomeric subunit is referred to as immunogenically neutral. One embodiment of the present invention is an ENV-SA fusion protein comprising an ENV protein of the present invention joined to an amino acid sequence at least 80%, at least 85%, at least 90%, at least 95%, and at least 97% identical to the amino acid sequence of a monomeric SA protein subunit that is responsible for directing self-assembly of the monomeric ferritin subunits into a nanoparticle, wherein the ENV-SA fusion protein is capable of self-assembling into nanoparticles. In one embodiment, the ENV-SA fusion protein comprises a polypeptide sequence identical in sequence to a monomeric SA protein subunit selected from the group consisting of ferritin, encapsulin, sulfur oxygenase reductase, lumazine synthase and pyruvate dehydrogenase complex (PDC) dihydrolipoamide acetyltransferase (E2). One embodiment of the present invention is an ENV-SA fusion protein comprising an ENV protein of the present invention joined to an amino acid sequence at least 80%, at least 85%, at least 90%, at least 95%, and at least 97% identical to the amino acid sequence of a monomeric SA protein subunit selected from the group consisting of ferritin, encapsulin, sulfur oxygenase reductase, lumazine synthase and pyruvate dehydrogenase complex (PDC) dihydrolipoamide acetyltransferase (E2), wherein the ENV-SA fusion protein is capable of self-assembling into nanoparticles. One embodiment of the present invention is an ENV-SA fusion protein comprising an ENV protein of the present invention joined to an amino acid sequence at least 80%, at least 85%, at least 90%, at least 95%, and at least 97% identical to a sequence selected from the group consisting of SEQ ID NO:2, SEQ ID NO:5 SEQ ID NO:8, SEQ ID NO:11, SEQ ID NO:14,SEQ ID NO:17, SEQ ID NO:20, SEQ ID NO:23, SEQ ID NO:26 and SEQ ID NO:29,wherein the ENV-SA fusion protein is capable of self-assembling into nanoparticles. One embodiment of the present invention is an ENV-SA fusion protein comprising an ENV protein of the present invention joined to an amino acid sequence at least 80%, at least 85%, at least 90%, at least 95%, and at least 97% identical to amino acid 5-167 from SEQ ID NO:2, SEQ ID NO:5 or SEQ ID NO:8, wherein the ENV-SA fusion protein is capable of self-assembling into nanoparticles.

In some embodiments, it may be useful to engineer mutations into the amino acid sequences of proteins of the present invention. For example, it may be useful to alter sites such as enzyme recognition sites or glycosylation sites in the monomeric ferritin subunit, the trimerization domain, or linker sequences, in order to give the fusion protein beneficial properties (e.g., stability, solubility, half-life, mask portions of the protein from immune surveillance). For example, it is known that the monomeric subunit of ferritin is not glycosylated naturally. However, it can be glycosylated if it is expressed as a secreted protein in mammalian or yeast cells. Thus, in one embodiment, potential N-linked glycosylation sites in the amino acid sequences from the monomeric ferritin subunit are mutated so that the mutated ferritin subunit sequences are no longer glycosylated at the mutated site.

According to the present invention, the EBV envelope protein portion of ENV-SA fusion proteins of the present invention can be from any EBV virus, so long as the ENV-SA fusion protein elicits an immune response against Epstein-Barr virus. Thus, one embodiment of the preset invention is an ENV-SA fusion protein comprising a self-assembly (SA) protein of the present invention joined to an amino acid sequence from an EBV envelope protein, wherein the ENV-SA fusion protein is capable of self-assembling into nanoparticles. One embodiment of the preset invention is an ENV-SA fusion protein comprising a self-assembly (SA) protein of the present invention joined to an amino acid sequence from a Type I EBV envelope protein, wherein the ENV-SA fusion protein is capable of self-assembling into nanoparticles. One embodiment of the preset invention is an ENV-SA fusion protein comprising a self-assembly (SA) protein of the present invention joined to an amino acid sequence from a Type II EBV envelope protein, wherein the ENV-SA fusion protein is capable of self-assembling into nanoparticles. One embodiment of the preset invention is an ENV-SA fusion protein comprising a SA protein of the present invention joined to an amino acid sequence from an EBV ENV protein listed in Table 2. One embodiment of the preset invention is an ENV-SA fusion protein comprising a SA protein of the present invention joined to an amino acid sequence from a protein selected from the group consisting of EBV gp350 protein, EBV gH protein, EBV gL protein and EBV gp42 protein. One embodiment of the present invention is an ENV-SA fusion protein comprising a SA protein of the present invention joined to an amino acid sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68 and SEQ ID NO:132.

Preferred ENV envelope proteins to use in constructing ENV-SA fusion proteins of the present invention are those that elicit an immune response against Epstein-Barr virus. Even more preferred EBV ENV proteins are those that are capable of eliciting antibodies to EBV. One embodiment of the present invention is an ENV-SA fusion protein that elicits antibodies to a Type I or Type II Epstein-Barr virus. One embodiment of the present invention is an ENV-SA fusion protein that elicits antibodies to an EBV ENV protein listed in Table 2. Preferred antibodies elicited by ENV-SA fusion proteins of the present invention are those that neutralize an Epstein-Barr virus. Thus, one embodiment of the present invention is an ENV-SA fusion protein that elicits neutralizing antibodies to a Type I or Type II EBV.

Neutralizing antibodies elicited by an ENV-SA fusion protein of the present invention can neutralize viral infections by affecting any step in the life cycle of the virus. Thus, in one embodiment of the present invention, an ENV-SA fusion protein elicits neutralizing antibodies that prevent EBV from attaching to the host cell. In one embodiment of the present invention, an ENV-SA fusion protein elicits neutralizing antibodies that prevent fusion of the viral envelope with the host cell membrane.

It will be understood by those skilled in the art that particularly useful ENV-SA proteins of the present invention are those comprising an immunogenic portion of an EBV envelope protein. Thus, one embodiment of the present invention is an ENV-SA fusion protein comprising a SA protein of the present invention joined to at least one immunogenic portion of an EBV ENV protein. One embodiment of the present invention is an ENV-SA protein comprising a SA protein of the present invention joined to at least one immunogenic portion of an ENV protein selected from the group consisting of EBV gp350 protein, EBV gH protein, EBV gL protein and EBV gp42 protein. One embodiment of the present invention is an ENV-SA protein comprising a SA protein of the present invention joined to at least one immunogenic portion of an ENV protein from the ENV proteins listed in Table 2. In one embodiment, an ENV-SA fusion protein comprising an immunogenic portion of an ENV protein elicits the production of neutralizing antibodies against EBV.

Immunogenic portions of proteins comprise epitopes, which are clusters of amino acid residues that are recognized by the immune system, thus eliciting an immune response. Such epitopes may consist of contiguous amino acids residues (i.e., amino acid residues that are adjacent to one another in the protein), or they may consist of non-contiguous amino acid residues (i.e., amino acid residues that are not adjacent one another in the protein) but which are in close special proximity in the finally folded protein. It is well understood by those skilled in the art that such epitopes require a minimum of six amino acid residues in order to be recognized by the immune system. Thus, one embodiment of the present invention is an ENV-SA fusion protein comprising an immunogenic portion from the ENV protein, wherein the immunogenic portion comprises at least one epitope.

It is known in the art that some variation in a protein sequence can be tolerated without significantly affecting the activity of the protein. Thus, one embodiment of the present invention is an ENV-SA fusion protein comprising a SA protein of the present invention joined to an amino acid sequence that is a variant of an ENV protein from a Type I or Type II Epstein-Barr virus. One embodiment of the present invention is an ENV-SA fusion protein comprising a ferritin protein of the present invention joined to an amino acid sequence at least about 80%, at least about 85%, at least about 90%, at least about 95%, at least about 97% or at least about 99% identical to the sequence of an ENV protein from a Type I or Type II Epstein-Barr virus, wherein the ENV-SA fusion protein elicits the production of neutralizing antibodies against EBV. One embodiment of the present invention is an ENV-SA fusion protein comprising an SA protein of the present invention joined to an amino acid sequence at least about 80%, at least about 85%, at least about 90%, at least about 95%, at least about 97% or at least about 99% identical to the sequence of an

ENV protein from those listed in Table 2, wherein the ENV-SA fusion protein elicits the production of neutralizing antibodies against EBV. One embodiment of the present invention is an ENV-SA fusion protein comprising an SA protein of the present invention joined to an amino acid sequence at least about 80%, at least about 85%, at least about 90%. at least about 95%, at least about 97% or at least about 99% identical to the sequence of an ENV protein selected from the group consisting of consisting of EBV gp350 protein. EBV gH protein. EBV gL protein and EBV gp42 protein. wherein the ENV-SA fusion protein elicits the production of neutralizing antibodies against EBV. One embodiment of the present invention is an ENV-SA fusion protein comprising an SA protein of the present invention joined to amino acid sequence at least about 80%, at least about 85%. at least about 90%, at least about 95%, at least about 97% or at least about 99% identical to a sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53,SEQ ID NO:56, SEQ ID NO:59, SEQ ID NO:62, SEQ ID NO:65, SEQ ID NO:68, SEQ ID NO:130, SEQ ID NO:132 and SEQ ID NO:136, wherein the ENV-SA fusion protein elicits the production of neutralizing antibodies against EBV. One embodiment of the present invention is an ENV-SA fusion protein comprising a SA protein of the present invention joined to amino acid sequence selected from the group consisting of SEQ ID NO:32, SEQ ID NO:35, SEQ ID NO:38, SEQ ID NO:41, SEQ ID NO:44, SEQ ID NO:47, SEQ ID NO 50, SEQ ID NO:53, SEQ ID NO:56, SEQ ID NO:59, SEQ ID NO:62, SEQ ID NO:65, SEQ ID NO:68, SEQ ID NO:130, SEQ ID NO:132 and SEQ ID NO:136.

One embodiment of the present invention is an ENV-SA fusion protein comprising an amino acid sequence at least 80%, at least about 85%, at least about 90%, at least about 95%, at least about 97% or at least about 99% identical to a sequence selected from the group consisting of SEQ ID NO:71, SEQ ID NO:74, SEQ ID NO:77, SEQ ID NO:80, SEQ ID NO:83, SEQ ID NO:86, SEQ ID NO:89, SEQ ID NO:92, SEQ ID NO:95, SEQ ID NO:98, SEQ ID NO:101, SEQ ID NO:104, SEQ ID NO:128, SEQ ID NO:130, SEQ ID NO:134 and SEQ ID NO:146. One embodiment of the present invention is an ENV-SA fusion protein comprising an amino acid sequence selected from the group consisting of SEQ ID NO:71, SEQ ID NO:74, SEQ ID NO:77, SEQ ID NO:80, SEQ ID NO:83, SEQ ID NO:86, SEQ ID NO:89, SEQ ID NO:92, SEQ ID NO:95, SEQ ID NO:98, SEQ ID NO:101, SEQ ID NO:104, SEQ ID NO:128, SEQ ID NO:130, SEQ ID NO:134 and SEQ ID NO:146.

It is known in the art that the EBV ENV proteins have various regions, or domains, each possessing specific activities. For example, EBV gp350 has an ectodomain that extends out from the viral membrane and comprises the receptor binding domain (RBD). Thus, it will be understood by those skilled in the art that ENV-SA fusion proteins of the present invention need not comprise the entire sequence of the EBV ENV protein. Instead, an ENV-SA fusion protein can comprise only those portions, regions, domains, and the like, that contain the necessary activities for practicing the present invention. For example, an ENV-SA fusion protein may contain only those amino acid sequences from the ENV protein that contain antigenic sites, epitopes, immunodominant epitopes, and the like.

One embodiment of the present invention is an ENV-SA fusion protein comprising a SA protein of the present invention joined to at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids from a protein from a Type I or Type II EBV, wherein the ENV-SA fusion protein elicits the production of neutralizing antibodies against EBV. One embodiment of the present invention is an ENV-SA fusion protein comprising a SA protein of the present invention joined to at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids from an ENV protein from those listed in Table 2, wherein the ENV-SA fusion protein elicits the production of neutralizing antibodies against EBV. One embodiment of the present invention is an ENV-SA fusion protein comprising a SA protein of the present invention joined to at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids from an ENV protein selected from the group consisting of consisting of EBV gp350 protein, EBV gH protein, EBV gL protein and EBV gp42 protein, wherein the ENV-SA fusion protein elicits the production of neutralizing antibodies against EBV. One embodiment of the present invention is an ENV-SA fusion protein comprising a SA protein of the present invention joined to at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids from a protein consisting of a sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:130, SEQ ID NO:132 and SEQ ID NO:136.

One embodiment of the present invention is an ENV-SA fusion protein comprising a SA protein of the present invention joined to at least one domain from an EBV gp350 protein, wherein the domain is selected from the group consisting of an ectodomain, an RDB domain, Domain I, Domain II and Domain III. According to the present invention, an ectodomain of an EBV gp350 protein refers to the portion of the gp350 protein that lies outside its transmembrane domain. One embodiment of the present invention is an ENV-SA fusion protein comprising a SA protein of the present invention joined a sequence selected from the group consisting of SEQ ID NO:35, SEQ ID NO:38, SEQ ID NO:41, SEQ ID NO:44, SEQ ID NO:47, SEQ ID NO:50, SEQ ID NO:53, SEQ ID NO:55 and SEQ ID NO:58.

ENV-SA proteins of the present invention are constructed by joining a SA protein of the present invention with an ENV protein of the present invention. In some embodiments, joining of the various proteins and/or domains can be done such that the sequences are directly linked. In other embodiments, it may be necessary to employ linkers (also referred to as a spacer sequences) between the various proteins and/or domains so that the so that they are in the proper orientation. More specifically, linker sequence can be inserted so that the ENV protein is positioned in such a way to maintain the ability to elicit an immune response against EBV. Linker sequences of the present invention comprise amino acids. Preferable amino acids to use are those having small side chains and/or those which are not charged. Such amino acids are less likely to interfere with proper folding and activity of the fusion protein. Accordingly, preferred amino acids to use in linker sequences, either alone or in combination are serine, glycine and alanine. Examples of such linker sequences include, but are not limited to, SGG, SGGG, GSG, GG, NGTGGSG and iterations thereof. Amino acids can be added or subtracted as needed. Those skilled in the art are capable of determining appropriate linker sequences for proteins of the present invention.

In accordance with the invention, suitable portions of the ENV protein can be joined to the SA protein by fusion with the N-terminal sequence, as an endocapsid product by fusion with the C-terminus, or a combination thereof. In one embodiment, the ENV portion of the fusion protein is joined to the N-terminal sequence of the SA portion of the fusion protein. In one embodiment, the ENV portion of the fusion protein is joined to the C-terminal sequence of the SA portion of the fusion protein.

The present inventors have also discovered that the production of nanoparticles of the present invention can be facilitated by using constructs expressing a fusion protein comprising multiple EBV proteins. Thus, one embodiment of the present invention is an ENV-SA fusion protein comprising a self-assembly (SA) protein of the present invention joined to amino acid sequences from two or more EBV envelope (ENV) proteins, wherein the ENV-SA fusion protein is capable of self-assembling into nanoparticles. In one embodiment, the two or more EBV envelope proteins are from Type I and/or Type II EBV. In one embodiment, the amino acid sequences are from two or more ENV envelope proteins listed in Table 2. In one embodiment, the ENV-SA fusion protein comprises a self-assembly protein joined to immunogenic portions from two or more EBV envelope proteins. In one embodiment, the two or more EBV envelope proteins are selected from the group consisting of EBV gp350 protein, EBV gH protein, EBV gL protein and EBV gp42 protein. In one embodiment, the two or more EBV envelope proteins comprise amino acid sequences selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68 and SEQ ID NO:132.

One embodiment of the present invention is an ENV-SA fusion protein comprising a self-assembly (SA) protein of the present invention joined to amino acid sequences from two or more an EBV envelope (ENV) proteins, wherein the ENV-SA fusion protein is capable of self-assembling into nanoparticles and wherein the two or more EBV envelope proteins are capable of eliciting antibodies to a Type I and/or Type II EBV. In one embodiment, the two or more EBV envelope proteins are capable of eliciting antibodies to at least one EBV envelope protein listed in Table 2. In a preferred embodiment the ENV-SA fusion protein elicits neutralizing antibodies to a Type I and/or a Type II EBV.

One embodiment of the present invention is an ENV-SA fusion protein comprising a self-assembly (SA) protein of the present invention joined to amino acid sequences from two or more an EBV envelope (ENV) proteins, wherein the ENV-SA fusion protein is capable of self-assembling into nanoparticles, and wherein the amino acid sequence from each of the two or more EBV envelope proteins comprises an immunogenic portion of an EBV envelope protein. In one embodiment, at least one of the immunogenic portions is capable of eliciting antibodies to a Type I and/or Type II EBV. In a preferred embodiment, the antibodies are neutralizing antibodies. In one embodiment, the immunogenic portions are from an EBV envelope protein listed in Table 2. In one embodiment, the immunogenic portions are from an EBV envelope protein comprising an amino acid sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56,SEQ ID NO:59, SEQ ID NO:62, SEQ ID NO:65, SEQ ID NO:68 and SEQ ID NO:132.

One embodiment of the present invention is an ENV-SA fusion protein comprising a self-assembly (SA) protein of the present invention joined to amino acid sequences from two or more an EBV envelope (ENV) proteins, wherein the two or more EBV envelope proteins are variants of EBV envelope proteins from a Type I and/or Type II EBV, and wherein the ENV-SA fusion protein is capable of self-assembling into nanoparticles. In one embodiment, the two or more EBV envelope proteins comprise amino acid sequences at least about 80%, at least about 85%, at least about 90%, at least about 95%, at least about 97% or at least about 99% identical to the sequence of an EBV envelope protein from a Type I and/or a Type II EBV. In one embodiment, the two or more EBV envelope proteins comprise amino acid sequences at least about 80%, at least about 85%, at least about 90%, at least about 95%, at least about 97% or at least about 99% identical to the sequence of an EBV envelope protein selected from the group consisting of EBV gp350 protein, EBV gH protein, EBV gL protein and EBV gp42 protein. In one embodiment, the two or more EBV envelope proteins comprise amino acid sequences at least about 80%, at least about 85%, at least about 90%, at least about 95%, at least about 97% or at least about 99% identical to the sequence of an EBV envelope protein listed in Table 2. In one embodiment, the two or more EBV envelope proteins comprise amino acid sequences at least about 80%, at least about 85%, at least about 90%, at least about 95%, at least about 97% or at least about 99% identical to a sequence selected from the group consisting of SEQ ID NO:32, SEQ ID NO:35, SEQ ID NO:38, SEQ ID NO:41, SEQ ID NO:44, SEQ ID NO:47, SEQ ID NO:50,SEQ ID NO:53, SEQ ID NO:56, SEQ ID NO:59, SEQ ID NO:62, SEQ ID NO:65, SEQ ID NO:68 and SEQ ID NO:132.

One embodiment of the present invention is an ENV-SA fusion protein comprising a self-assembly (SA) protein of the present invention joined to amino acid sequences from two or more EBV envelope (ENV) proteins, wherein each amino acid sequence from the two or more EBV envelope proteins is at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids in length, and wherein the ENV-SA fusion protein is capable of self-assembling into nanoparticles. In one embodiment, at least one of the amino acid sequences is capable of eliciting antibodies to a Type I and/or Type II EBV. In a preferred embodiment, the antibodies are neutralizing antibodies. In one embodiment, each amino acid sequence from the two or more EBV envelope proteins comprises at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids from an EBV envelope protein selected from the group consisting of EBV gH protein, EBV gL protein, EBV gp42 protein and EBV gp350) protein. In one embodiment, each amino acid sequence from the two or more EBV envelope proteins comprises at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids from an EBV envelope protein listed in Table 2. In one embodiment, each amino acid sequence from the two or more EBV envelope proteins comprises at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids from a protein having an amino acid sequence at least about 80%, at least about 85%, at least about 90%, at least about 95%, at least about 97% or at least about 99% identical to a sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68 and SEQ ID NO:132. In one embodiment, each amino acid sequence from the two or more EBV envelope proteins comprises at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids from a protein having an amino acid sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68 and SEQ ID NO:132.

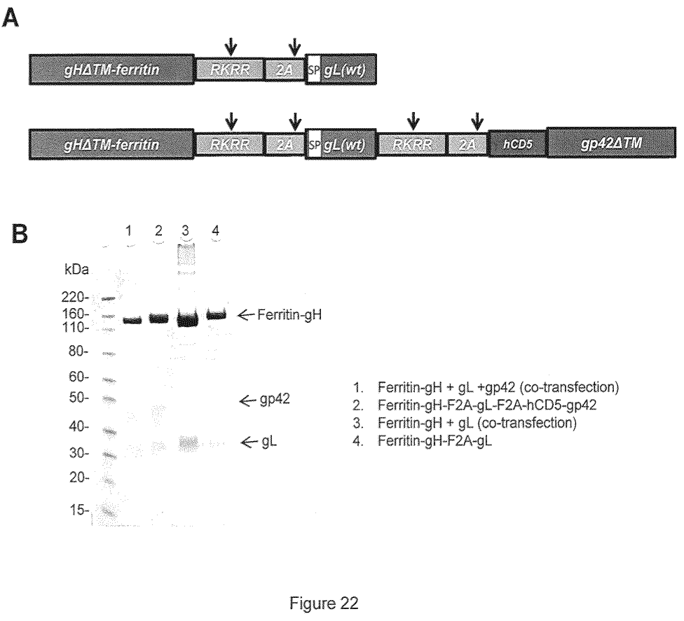

In certain embodiments, sequences within the fusion protein are directly joined. In alternative embodiments, it is useful to employ linkers, spacers or other types of sequences in order to obtain desired results. Such sequences can be inserted between specific fusion elements in order to, for example, maintain stoichiometry (or molecular ratio) of the final proteins, maintain proper orientation of domains in the final fusion protein, to facilitate transport of the final protein within or out of a cell, or to allow cleavage of the final protein. Thus, examples of useful sequences to utilize include, but are not limited to, linker sequences, spacer sequences, binding sequences, cleavage sequences, leader sequences and secretion signal sequences. Examples of constructs utilizing such sequences are shown in FIG. 22. Thus, one embodiment of the present invention is a ENV-SA fusion protein comprising one or more sequences selected from the group consisting of spacer sequences, binding sequences, cleavage sequences, leader sequences and secretion signal sequences. In one embodiment, the ENV-SA fusion protein comprises one or more protease cleavage sequence. In one embodiment, the cleavage site is a self-cleavage site. In one embodiment, the cleavage sequence is a picornavirus protease cleavage sequence. In one embodiment, the cleavage sequence is a furin cleavage sequence (Arg-Lys-Arg-Arg). In one embodiment, the ENV-SA fusion protein comprises a sequence that directs secretion of the protein. In one embodiment, the fusion protein comprises a leader sequence from human CD5. In one embodiment, the fusion protein comprises one or more sequences selected from the group consisting of SEQ ID NO:138, SEQ ID NO:140, RKRR and functional variants thereof.

One embodiment of the present invention is a ENV-SA fusion protein comprising a self-assembly (SA) protein of the present invention joined to amino acid sequences from two or more EBV envelope (ENV) proteins, wherein the fusion protein comprises at least one cleavage site positioned such that cleavage, including self-cleavage, at the cleavage site results in a SA-gH fusion protein. In one embodiment, the SA-gH fusion protein is capable of forming a dimer with EBVgL. In one embodiment, the SA-gH fusion protein is capable of forming a trimer with EBV gL and EBV gp42. In one embodiment, the ENV-SA fusion protein comprises an EBV gH protein and one or more of EBV gL and EBV gp42 protein. In one embodiment, the fusion protein comprises additional cleavage sites such that cleavage, including self-cleavage, of the fusion protein results in a SA-gH fusion protein and one or more of an EBV gL protein and EBV gp42 protein. One embodiment of the present invention is a fusion protein comprising an amino acid sequence at least about 80%, at least about 85%, at least about 90%, at least about 95%, at least about 97% or at least about 99% identical to a sequence selected from the group consisting of SEQ ID NO:142 and SEQ ID NO:144. One embodiment of the present invention is a fusion protein comprising SEQ ID NO:142 or SEQ ID NO:144.

Proteins of the present invention are encoded by nucleic acid molecules of the present invention. In addition, they are expressed by nucleic acid constructs of the present invention. As used herein a nucleic acid construct is a recombinant expression vector, i.e., a vector linked to a nucleic acid molecule encoding a protein such that the nucleic acid molecule can effect expression of the protein when the nucleic acid construct is administered to, for example, a subject or an organ, tissue or cell. The vector also enables transport of the nucleic acid molecule to a cell within an environment, such as, but not limited to, an organism, tissue, or cell culture. A nucleic acid construct of the present disclosure is produced by human intervention. The nucleic acid construct can be DNA, RNA or variants thereof. The vector can be a DNA plasmid, a viral vector, or other vector. In one embodiment, a vector can be a cytomegalovirus (CMV), retrovirus, adenovirus, adeno-associated virus, herpes virus, vaccinia virus, poliovirus, sindbis virus, or any other DNA or RNA virus vector. In one embodiment, a vector can be a pseudotyped lentiviral or retroviral vector. In one embodiment, a vector can be a DNA plasmid. In one embodiment, a vector can be a DNA plasmid comprising viral components and plasmid components to enable nucleic acid molecule delivery and expression. Methods for the construction of nucleic acid constructs of the present disclosure are well known. See, for example, Molecular Cloning: a Laboratory Manual. 3^{rd} edition, Sambrook et al. 2001 Cold Spring Harbor Laboratory Press, and Current Protocols in Molecular Biology, Ausubel et al. eds., John Wiley & Sons, 1994. In one embodiment, the vector is a DNA plasmid, such as a CMV/R plasmid such as CMV/R or CMV/R 8 KB (also referred to herein as CMV/R 8 kb). Examples of CMV/R and CMV/R 8 kb are provided herein. CMV/R is also described in U.S. Pat. No. 7,094,598 B2, issued Aug. 22, 2006.

As used herein, a nucleic acid molecule comprises a nucleic acid sequence that encodes a SA monomeric subunit, an ENV protein, and/or an ENV-ferritin SA protein of the present invention. A nucleic acid molecule can be produced recombinantly, synthetically, or by a combination of recombinant and synthetic procedures. A nucleic acid molecule of the disclosure can have a wild-type nucleic acid sequence or a codon-modified nucleic acid sequence to, for example, incorporate codons better recognized by the human translation system. In one embodiment, a nucleic acid molecule can be genetically-engineered to introduce, or eliminate, codons encoding different amino acids, such as to introduce codons that encode an N-linked glycosylation site. Methods to produce nucleic acid molecules of the disclosure are known in the art, particularly once the nucleic acid sequence is know. It is to be appreciated that a nucleic acid construct can comprise one nucleic acid molecule or more than one nucleic acid molecule. It is also to be appreciated that a nucleic acid molecule can encode one protein or more than one protein.

Preferred nucleic acid molecules are those that encode a SA monomeric subunit, an ENV protein, and/or an ENV-SA fusion protein comprising a monomeric subunit of a SA protein joined to an EBV ENV protein. Thus, one embodiment of the present invention is a nucleic acid molecule comprising a nucleic acid sequence encoding a protein that comprises a monomeric subunit of a SA protein joined to an EBV ENV protein. In one embodiment, the monomeric subunit of an SA protein comprises an amino acid sequence at least 80%, at least 85%, at least 90%, at least 92%, at least 94%, at least 96%, at least 98% or at least 99% identical to a sequence selected from the group consisting of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26 and SEQ ID NO:29. In one embodiment, the monomeric subunit comprises an amino acid sequence selected from the group consisting of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26 and SEQ ID NO:29. In one embodiment the EBV ENV protein comprises an amino acid sequence at least 80%, at least 85%, at least 90%, at least 92%, at least 94%, at least 96%, at least 98% or at least 99% identical to a sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:130, SEQ ID NO:132 and SEQ ID NO:136. In one embodiment the EBV ENV protein comprises a sequence selected from the group consisting of SEQ ID NO:32, SEQ ID NO:35, SEQ ID NO:38, SEQ ID NO:41, SEQ ID NO:44, SEQ ID NO:47. SEQ ID NO:50, SEQ ID NO:53, SEQ ID NO:56, SEQ ID NO:59, SEQ ID NO:62, SEQ ID NO:65, SEQ ID NO:68, SEQ ID NO:130, SEQ ID NO:132 and SEQ ID NO:136. In one embodiment the EBV ENV protein comprises at least 25 amino acids, at least 50 amino acids, at least 75 amino acids, at least 100 amino acids, at least 150 amino acids, or at least 200 amino acids from a sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65,SEQ ID NO:68, SEQ ID NO:130, SEQ ID NO:132 and SEQ ID NO:136.

One embodiment of the present invention is a nucleic acid molecule comprising a nucleic sequence encoding a protein comprising an amino acid sequence at least 80%, at least 85%. at least 90%, at least 92%, at least 94%, at least 96%, at least 98% or at least 99% identical to a sequence selected from the group consisting of SEQ ID NO:71, SEQ ID NO:74, SEQ ID NO:77, SEQ ID NO:80, SEQ ID NO:83, SEQ ID NO:86, SEQ ID NO:89.SEQ ID NO:92, SEQ ID NO:95, SEQ ID NO:98, SEQ ID NO:101, SEQ ID NO:104. SEQ ID NO:128, SEQ ID NO:130, SEQ ID NO:134 and SEQ ID NO:146. One embodiment of the present invention is a nucleic acid molecule comprising a nucleic sequence encoding an amino acid sequence selected from the group consisting of SEQ ID NO:71, SEQ ID NO:74,SEQ ID NO:77, SEQ ID NO:80, SEQ ID NO:83, SEQ ID NO:86, SEQ ID NO:89, SEQ ID NO:92, SEQ ID NO:95, SEQ ID NO:98, SEQ ID NO:101, SEQ ID NO:104, SEQ ID NO 128, SEQ ID NO:130, SEQ ID NO:134 and SEQ ID NO:146.

Also embodied in the present invention are nucleic acid sequences that are variants of nucleic acid sequence encoding protein of the present invention. Such variants include nucleotide insertions, deletions, and substitutions, so long as they do not affect the ability of fusion proteins of the present invention to self-assemble into nanoparticles, or significantly affect the ability of the EBV envelope portion of fusion proteins to elicit an immune response to an Epstein-Barr virus. Thus, one embodiment of the present invention is a nucleic acid molecule encoding a fusion protein of the present invention, wherein the monomeric subunit of the SA protein is encoded by a nucleotide sequence at least 85%, at least 90%, at least 95%, or at least 97% identical to a nucleotide sequence selected from the group consisting of SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:2 and SEQ ID NO:28.One embodiment of the present invention is a nucleic acid molecule encoding an ENV-SA fusion protein of the present invention, wherein the ENV protein is encoded by a nucleotide sequence at least 85%. at least 90%, at least 95%, at least 97% identical or at least 99% identical to a nucleic acid sequence encoding an amino acid sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:130, SEQ ID NO:132and SEQ ID NO:136.

One embodiment of the present invention is a nucleic acid molecule comprising a nucleic acid sequence at least about 80%, at least about 85%, at least about 90%, at least about 95%, at least about 97% or at least about 99% identical to a nucleic acid sequence selected from the group consisting of SEQ ID NO:10, SEQ ID NO:13 and SEQ ID NO:16. One embodiment of the present invention is a nucleic acid molecule comprising a nucleic acid sequence selected from the group consisting of SEQ ID NO:10, SEQ ID NO:13 and SEQ ID NO:16.

One embodiment of the present invention is a nucleic acid molecule comprising a nucleic acid sequence at least about 80%, at least about 85%, at least about 90%, at least about 95%, at least about 97% or at least about 99% identical to a nucleic acid sequence selected from the group consisting of SEQ ID NO:70, SEQ ID NO:73, SEQ ID NO:76, SEQ ID NO:79, SEQ ID NO:82, SEQ ID NO:85, SEQ ID NO:88 and SEQ ID NO:92. One embodiment of the present invention is a nucleic acid molecule comprising a nucleic acid sequence selected from the group consisting of SEQ ID NO:70, SEQ ID NO:73, SEQ ID NO:76, SEQ ID NO:79, SEQ ID NO:82, SEQ ID NO:85, SEQ ID NO:88, SEQ ID NO:91, SEQ ID NO:94, SEQ ID NO:97, SEQ ID NO:100, SEQ ID NO:103, SEQ ID NO:127, SEQ ID NO:129, SEQ ID NO:133 and SEQ ID NO:145.

One embodiment of the present invention is a nucleic acid molecule encoding an ENV-SA fusion protein comprising a self-assembly (SA) protein of the present invention joined to amino acid sequences from two or more an EBV envelope (ENV) proteins, wherein the ENV-SA fusion protein is capable of self-assembling into nanoparticles. In one embodiment, the two or more EBV envelope proteins are from Type I and/or Type II EBV. In one embodiment, the amino acid sequences are from two or more ENV envelope proteins listed in Table 2. In one embodiment, the ENV-SA fusion protein comprises a self-assembly protein joined to immunogenic portions from two or more EBV envelope proteins. In one embodiment, the two or more EBV envelope proteins are selected from the group consisting of EBV gp350 protein, EBV gH protein, EBV gL protein and EBV gp42 protein. In one embodiment, the two or more EBV envelope proteins comprise amino acid sequences selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68 and SEQ ID NO:132.

One embodiment of the present invention is nucleic acid molecule encoding a ENV-SA fusion protein comprising a self-assembly (SA) protein of the present invention joined to amino acid sequences from two or more EBV envelope (ENV) proteins, wherein the ENV-SA fusion protein is capable of self-assembling into nanoparticles and wherein the two or more EBV envelope proteins are capable of eliciting antibodies to a Type I and/or Type II EBV. In one embodiment, the two or more EBV envelope proteins are capable of eliciting antibodies to at least one EBV envelope protein listed in Table 2. In a preferred embodiment the ENV-SA fusion protein elicits neutralizing antibodies to a Type I and/or a Type II EBV.

One embodiment of the present invention is a nucleic acid molecule encoding a ENV-SA fusion protein comprising a self-assembly (SA) protein of the present invention joined to amino acid sequences from two or more EBV envelope (ENV) proteins, wherein the ENV-SA fusion protein is capable of self-assembling into nanoparticles, and wherein the amino acid sequence from each of the two or more EBV envelope proteins comprises an immunogenic portion of an EBV envelope protein. In one embodiment, at least one of the immunogenic portions is capable of eliciting antibodies to a Type I and/or Type II EBV. In a preferred embodiment, the antibodies are neutralizing antibodies. In one embodiment, the immunogenic portions are from an EBV envelope protein listed in Table 2. In one embodiment, the immunogenic portions are from an EBV envelope protein comprising an amino acid sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68 and SEQ ID NO:132.

One embodiment of the present invention is a nucleic acid molecule encoding a ENV-SA fusion protein comprising a self-assembly (SA) protein of the present invention joined to amino acid sequences from two or more EBV envelope (ENV) proteins, wherein the two or more EBV envelope proteins are variants of EBV envelope proteins from a Type I and/or Type II EBV, and wherein the ENV-SA fusion protein is capable of self-assembling into nanoparticles. In one embodiment, the two or more EBV envelope proteins comprise amino acid sequences at least about 80%, at least about 85%, at least about 90%, at least about 95%, at least about 97% or at least about 99% identical to the sequence of an EBV envelope protein from a Type I and/or a Type II EBV. In one embodiment, the two or more EBV envelope proteins comprise amino acid sequences at least about 80%, at least about 85%, at least about 90%, at least about 95%, at least about 97% or at least about 99% identical to the sequence of an EBV envelope protein selected from the group consisting of EBV gp350 protein, EBV gH protein, EBV gL protein and EBV gp42 protein. In one embodiment, the two or more EBV envelope proteins comprise amino acid sequences at least about 80%, at least about 85%, at least about 90%, at least about 95%, at least about 97% or at least about 99% identical to the sequence of an EBV envelope protein listed in Table 2. In one embodiment, the two or more EBV envelope proteins comprise amino acid sequences at least about 80%, at least about 85%, at least about 90%, at least about 95%, at least about 97% or at least about 99% identical to a sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68 and SEQ ID NO:132.

One embodiment of the present invention is a nucleic acid molecule encoding a ENV-SA fusion protein comprising a self-assembly (SA) protein of the present invention joined to amino acid sequences from two or more EBV envelope (ENV) proteins, wherein each amino acid sequence from the two or more EBV envelope proteins is at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids in length, and wherein the ENV-SA fusion protein is capable of self-assembling into nanoparticles. In one embodiment, at least one of the amino acid sequences is capable of eliciting antibodies to a Type I and/or Type II EBV. In a preferred embodiment, the antibodies are neutralizing antibodies. In one embodiment, each amino acid sequence from the two or more EBV envelope proteins comprises at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids from an EBV envelope protein selected from the group consisting of EBV gH protein, EBV gL protein, EBV gp42 protein and EBV gp350 protein. In one embodiment, each amino acid sequence from the two or more EBV envelope proteins comprises at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids from an EBV envelope protein listed in Table 2. In one embodiment, each amino acid sequence from the two or more EBV envelope proteins comprises at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids from a protein having an amino acid sequence at least about 80%, at least about 85%, at least about 90%, at least about 95%, at least about 97% or at least about 99% identical to a sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68 and SEQ ID NO:132. In one embodiment, each amino acid sequence from the two or more EBV envelope proteins comprises at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids from a protein having an amino acid sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68 and SEQ ID NO:132.

One embodiment of the present invention is a nucleic acid molecule comprising a nucleotide sequence at least about 80%, at least about 85%, at least about 90%, at least about 95%, at least about 97% or at least about 99% identical to a sequence selected from the group consisting of SEQ ID NO:141 and SEQ ID NO:143. One embodiment of the present invention is a nucleic acid molecule comprising SEQ ID NO:141 or SEQ ID NO:143.

Also encompassed by the present invention are expression systems for producing fusion proteins of the present invention. In one embodiment, nucleic acid molecules of the present invention are operationally linked to a promoter. As used herein, operationally linked means that proteins encoded by the linked nucleic acid molecules can be expressed when the linked promoter is activated. Promoters useful for practicing the present invention are known to those skilled in the art. One embodiment of the present invention is a recombinant cell comprising a nucleic acid molecule of the present invention. One embodiment of the present invention is a recombinant virus comprising a nucleic acid molecule of the present invention.

As indicated above, the recombinant production of the ENV-SA fusion proteins of the present invention can take place using any suitable conventional recombinant technology currently known in the field. For example, molecular cloning of a construct expressing a fusion protein, such as a SA protein of the present invention with a suitable protein such as a recombinant EBV ENV protein, can be carried out via expression in E. coli. The construct may then be transformed into protein expression cells, grown to suitable size, and induced to produce the fusion protein.

As has been described, because ENV-SA fusion proteins of the present invention comprise a monomeric self-assembly (SA) protein, they can self-assemble. According to the present invention, the supramolecule resulting from such self-assembly is referred to as an ENV-expressing, SA protein-based nanoparticle. For ease of discussion, the ENV-expressing, SA protein-based nanoparticle will simply be referred to as a, or the, nanoparticle (np). Nanoparticles of the present invention comprise fusion proteins comprising a SA monomeric subunit joined to an EBV ENV protein. Such nanoparticles display at least a portion of the ENV protein on their surface. In such a construction, the ENV protein is accessible to the immune system and thus can elicit an immune response. Thus, one embodiment of the present invention is a nanoparticle comprising an ENV-SA fusion protein, wherein the fusion protein comprises a monomeric SA subunit joined to an EBV ENV protein. In one embodiment, the nanoparticle is an octahedron. In one embodiment, the ENV protein is capable of eliciting neutralizing antibodies to EBV. In one embodiment, the monomeric SA subunit comprises at least 50 amino acids, at least 100 amino acids, or at least 150 amino acids from an amino acid sequence selected from the group consisting of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26 and SEQ ID NO 29. In one embodiment, the monomeric SA subunit comprises an amino acid sequence at least 85%, at least 90%, at least 95%, at least 97% at least 99% identical to an amino acid sequence selected from the group consisting of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26 and SEQ ID NO:29. In one embodiment, the monomeric SA subunit comprises an amino acid sequence selected from the group consisting of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26 and SEQ ID NO:29.

In one embodiment, the ENV protein comprises at least one epitope from an EBV ENV protein listed in Table 2. In one embodiment, the ENV protein comprises at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids from an ENV protein listed in Table 2. In one embodiment, the ENV protein comprises at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids from a protein consisting of a sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:130, SEQ ID NO:132 and SEQ ID NO:136. In one embodiment, the ENV protein comprises a sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68.

In one embodiment, the ENV protein comprises an amino acid sequence at least 80%, at least 85%, at least 90%, at least 95%, at least 97%, at least 99% identical to the sequence of an ENV protein listed in Table 2. In one embodiment, the ENV protein comprises an amino acid sequence at least 80%, at least 85%, at least 90%, at least 95%, at least 97%, at least 99% identical to a protein sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:130, SEQ ID NO:132 and SEQ ID NO:136.

In one embodiment, the ENV-SA fusion protein comprises an amino acid sequence at least 80%, at least 85%, at least 90%, at least 95%, at least 97%, at least 99% identical to a protein sequence selected from the group consisting of SEQ ID NO:71, SEQ ID NO:74,SEQ ID NO:77, SEQ ID NO:80, SEQ ID NO:83, SEQ ID NO:86, SEQ ID NO:89, SEQ ID NO:92, SEQ ID NO:95, SEQ ID NO:98, SEQ ID NO:101, SEQ ID NO:104, SEQ ID NO:128, SEQ ID NO:130, SEQ ID NO:134 and SEQ ID NO:146. In one embodiment, the ENV-SA fusion protein comprises an amino acid sequence selected from the group consisting of SEQ ID NO:71, SEQ ID NO:74, SEQ ID NO:77, SEQ ID NO:80, SEQ ID NO:83, SEQ ID NO:86, SEQ ID NO:89, SEQ ID NO:92, SEQ ID NO:95, SEQ ID NO:98, SEQ ID NO:101, SEQ ID NO:104, SEQ ID NO:128, SEQ ID NO:130, SEQ ID NO:134 and SEQ ID NO:146.

Nanoparticles of the present invention may comprise more than one type of fusion protein. That is, a nanoparticle of the present invention may comprise at least two types of fusion proteins, each of which comprises amino acid sequences from different EBV ENV proteins (e.g., gp350) and gH). Moreover, the different types of fusion proteins may comprise amino acid sequences from the same or different SA protein (i.e., ferritin and/or encapsulin). Furthermore, in addition to at least one ENV-SA fusion protein, nanoparticles of the present invention may comprise proteins that are not fused to a SA protein. For example, in addition to comprising an ENV-SA fusion protein (e.g., EBV gH-ferritin protein) a nanoparticle of the present invention may also comprise one or more proteins comprising an amino acid sequence from other EBV ENV proteins, or portions or variants thereof. Examples of such proteins include, but are not limited to gp350, gH, gL and gp42. Such additional one or more proteins may, but need not, form a complex with each other or with the ENV-SA fusion protein.

Because ENV-SA fusion proteins and nanoparticles of the present invention can elicit an immune response to an Epstein-Barr virus, they can be used as vaccines to protect individuals against infection by EBV. According to the present invention a vaccine can be an ENV-SA fusion protein, or a nanoparticle of the present invention. Thus, one embodiment of the present invention is a vaccine comprising an ENV-SA fusion protein or a nanoparticle of the present invention. Vaccines of the present invention can also contain other components such as adjuvants, buffers and the like. Although any adjuvant can be used, preferred embodiments can contain: chemical adjuvants such as aluminum phosphate, benzyalkonium chloride, ubenimex, and QS21: genetic adjuvants such as the IL-2 gene or fragments thereof, the granulocyte macrophage colony-stimulating factor (GM-CSF) gene or fragments thereof, the IL-18 gene or fragments thereof, the chemokine (C-C motif) ligand 21 (CCL21) gene or fragments thereof, the IL-6 gene or fragments thereof, CpG, LPS, TLR agonists, and other immune stimulatory genes: protein adjuvants such IL-2 or fragments thereof, the granulocyte macrophage colony-stimulating factor (GM-CSF) or fragments thereof, IL-18 or fragments thereof, the chemokine (C-C motif) ligand 21 (CCL21) or fragments thereof, IL-6 or fragments thereof, CpG, LPS, TLR agonists and other immune stimulatory cytokines or fragments thereof: lipid adjuvants such as cationic liposomes, N3 (cationic lipid), monophosphoryl lipid A (MPL1): other adjuvants including cholera toxin, enterotoxin, Fms-like tyrosine kinase-3 ligand (Flt-3L), bupivacaine, marcaine, and levamisole.

One embodiment of the disclosure is a SA protein-based nanoparticle vaccine that includes more than one EBV ENV protein. Such a vaccine can include a combination of different EBV envelope proteins, either in a single nanoparticle or as a mixture of nanoparticles, at least two of which have unique EBV ENV proteins. A multivalent vaccine can comprise as many EBV envelope proteins as necessary in order to result in production of the desired immune response. In one embodiment, the vaccine comprises ENV proteins from at least two different Types of EBV (bi-valent). In one embodiment, the vaccine comprises a ENV protein from at least three different Epstein-Barr viruses (tri-valent).

One embodiment of the present invention is a method to vaccinate an individual against EBV, the method comprising administering a nanoparticle to an individual such that an immune response against EBV is produced in the individual, wherein the nanoparticle comprises a monomeric subunit from an SA protein of the present invention joined to an EBV envelope protein of the present invention protein, and wherein the nanoparticle displays the EBV envelope on its surface. In one embodiment, the nanoparticle is a monovalent nanoparticle. In one embodiment, the nanoparticle is multivalent nanoparticle. Another embodiment of the present invention is a method to vaccinate an individual against infection with EBV, the method comprising:

a) obtaining a nanoparticle comprising monomeric subunits, wherein the monomeric subunits comprise an SA protein joined to an EBV ENV protein, and wherein the nanoparticle displays the EBV ENV protein on its surface; and,

b) administering the nanoparticle to an individual such that an immune response against EBV is produced.

One embodiment of the present invention is a method to vaccinate an individual against EBV, the method comprising administering a vaccine of the embodiments to an individual such that an immune response against EBV is produced in the individual, wherein the vaccine comprises at least one nanoparticle comprising a monomeric subunit of an SA protein of the present invention joined to an EBV envelope protein of the present invention protein, and wherein the nanoparticle displays the EBV ENV protein on its surface. In one embodiment, the vaccine is a nanoparticle. In one embodiment, the vaccine is a monovalent vaccine. In one embodiment, the vaccine is multivalent vaccine. Another embodiment of the present invention is a method to vaccinate an individual against infection with EBV, the method comprising:

a) obtaining a vaccine comprising at least one nanoparticle comprising an ENV-SA fusion protein, wherein the fusion protein comprises an SA protein joined to an EBV ENV protein, and wherein the nanoparticle displays the EBV ENV on its surface; and,

b) administering the vaccine to an individual such that an immune response against EBV is produced.

In one embodiment, the nanoparticle is a monovalent nanoparticle. In one embodiment, the nanoparticle is multivalent nanoparticle. In one embodiment, the nanoparticle is an octahedron. In one embodiment, the EBV ENV protein is capable of eliciting neutralizing antibodies to EBV. In one embodiment, the SA portion of the fusion protein comprise at least 50 amino acids, at least 100 amino acids, or at least 150 amino acids from an amino acid sequence selected from the group consisting of SEQ ID NO:2.SEQ ID NO:5, SEQ ID NO:8, SEQ ID NO:11, SEQ ID NO:14, SEQ ID NO:17, SEQ ID NO:20, SEQ ID NO:23, SEQ ID NO:26 and SEQ ID NO:29. In one embodiment, the SA portion of the fusion protein comprises an amino acid sequence at least 85%, at least 90%, at least 95%, at least 97% at least 99% identical to an amino acid sequence selected from the group consisting of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26 and SEQ ID NO:29. In one embodiment, the ENV portion of the fusion protein comprises at least one epitope from an ENV protein listed in Table 2. In one embodiment, the ENV portion of the fusion protein comprises at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids from an ENV listed in Table 2. In one embodiment, the ENV portion of the fusion protein comprises at least 25amino acids, at least 50 amino acids, at least 75 amino acids, at least 100 amino acids, at least 150 amino acids, at least 200 amino acids, at least 300 amino acids, at least 400 amino acids, or at least 500 amino acids from a protein consisting of a sequence selected from the group consisting of SEQ ID NO:32, SEQ ID NO:35, SEQ ID NO:38, SEQ ID NO:41, SEQ ID NO: 44, SEQ ID NO:47, SEQ ID NO:50, SEQ ID NO:53, SEQ ID NO:56, SEQ ID NO 59, SEQ ID NO:62, SEQ ID NO:65, SEQ ID NO:68, SEQ ID NO:130, SEQ ID NO:132and SEQ ID NO:136. In one embodiment, the ENV portion of the fusion protein comprises an amino acid sequence at least 80%, at least 85%, at least 90%, at least 95%, at least 97%, at least 99% identical to the sequence of an ENV protein listed in Table 2. In one embodiment, the ENV portion of the fusion protein comprises an amino acid sequence at least 80%, at least 85%, at least 90%, at least 95%, at least 97%, at least 99% identical to a sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53,SEQ ID NO:56, SEQ ID NO:59, SEQ ID NO:62, SEQ ID NO:65, SEQ ID NO:68, SEQ ID NO:130, SEQ ID NO:132 and SEQ ID NO:136. In one embodiment, the ENV-SA fusion protein comprises an amino acid sequence at least 80%, at least 85%, at least 90%, at least 95%, at least 97%, at least 99% identical to a protein sequence selected from the group consisting of SEQ ID NO:71, SEQ ID NO:74, SEQ ID NO:77, SEQ ID NO:80, SEQ ID NO:83, SEQ ID NO:86, SEQ ID NO:89, SEQ ID NO:92, SEQ ID NO:95, SEQ ID NO:98, SEQ ID NO:101, SEQ ID NO:104, SEQ ID NO:128, SEQ ID NO:130 and SEQ ID NO:134. In one embodiment, the ENV-SA fusion protein comprises an amino acid sequence selected from the group consisting of SEQ ID NO:71, SEQ ID NO:74, SEQ ID NO:77, SEQ ID NO:80, SEQ ID NO:83, SEQ ID NO:86, SEQ ID NO:89, SEQ ID NO:92, SEQ ID NO:95, SEQ ID NO:98, SEQ ID NO:101, SEQ ID NO:104, SEQ ID NO:128, SEQ ID NO:130 and SEQ ID NO:134.

Vaccines of the present invention can be used to vaccinate individuals using a prime/boost protocol. Such a protocol is described in U.S. Patent Publication No. 20110177122, which is incorporated herein by reference in its entirety. In such a protocol, a first vaccine composition may be administered to the individual (prime) and then after a period of time, a second vaccine composition may be administered to the individual (boost). Administration of the boosting composition may be performed days, weeks or months after administration of the priming composition, preferably about 10 days, about two weeks, about three weeks, about 4 weeks, about 8 weeks, about 16 weeks, about 20 weeks, about 24 weeks, about 28 weeks, or about 32 weeks. In one embodiment, the boosting composition is formulated for administration about 1 week, about 2 weeks, about 3 weeks, about 4 weeks, about 5 weeks, about 6 weeks, about 7 weeks, about 8 weeks, about 9 weeks, about 16 weeks, about 20 weeks, about 24 weeks, about 28 weeks, or about 32 weeks after administration of the priming composition. In one embodiment, a second boosting composition (i.e., third vaccine composition) is administered at some period of time following administration of the first boosting composition. For example, a second boosting composition may be administered at a time about 8 weeks, about 9 weeks, about 10 weeks, about 12 weeks, about 16 weeks, about 20 weeks, about 24 weeks, about or 32 weeks after administration of the priming composition. In one embodiment, a second boosting composition is administered 6 months after administration of the priming composition. As used herein, and with specific regard to the timing of administration of a vaccine composition, the term about refers to a variation of no more than 10%. Thus for example, about 10 days specifies a time period of 9-11 days. Likewise, for example, about 6 months specifies a time period of 162-196 days.

The first and second vaccine compositions can be, but need not be, the same composition. Thus, in one embodiment of the present invention, the step of administering the vaccine comprises administering a first vaccine composition, and then at a later time, administering a second vaccine composition. In one embodiment, the first vaccine composition comprises a nanoparticle comprising an ENV-SA fusion protein of the present invention. In one embodiment, the first vaccine composition comprises a nanoparticle comprising EBV ENV protein. In one embodiment, the ENV of the first vaccine composition comprises an amino acid sequence at least about 80% identical. at least 85% identical, at least 90% identical, at least 95% identical, at least 97% identical or at least 99% identical to an amino acid sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:130, SEQ ID NO:132, SEQ ID NO:136 and SEQ ID NO:146.In one embodiment, the first vaccine composition comprises an ENV-SA fusion protein comprising an amino acid sequence at least 80% identical, at least 85% identical, at least 90% identical, at least 95% identical, at least 97% identical or at least 99% identical to a sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:130, SEQ ID NO:132, SEQ ID NO:136 and SEQ ID NO:146, wherein the nanoparticle elicits an immune response against EBV. In one embodiment, the first vaccine composition comprises an ENV-SA fusion protein comprising an amino acid sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, and SEQ ID NO:68. In one embodiment, second vaccine composition comprises a nanoparticle comprising an ENV-SA fusion protein of the present invention. In one embodiment, the individual has been exposed to EBV. As used herein, the terms exposed, exposure, and the like, indicate the subject has come in contact with a person of animal that is known to be infected with an EBV. Vaccines of the present invention may be administered using techniques well known to those in the art. Techniques for formulation and administration may be found, for example, in “Remington's Pharmaceutical Sciences”, 18^{th} ed., 1990, Mack Publishing Co., Easton, PA. Vaccines may be administered by means including, but not limited to, traditional syringes, needleless injection devices, or microprojectile bombardment gene guns. Suitable routes of administration include, but are not limited to, parenteral delivery, such as intramuscular, intradermal, subcutaneous, intramedullary injections, as well as, intrathecal, direct intraventricular, intravenous, intraperitoneal, intranasal, or intraocular injections, just to name a few. For injection, the compounds of one embodiment of the invention may be formulated in aqueous solutions, preferably in physiologically compatible buffers such as Hanks' solution, Ringer's solution, or physiological saline buffer.

In one embodiment, vaccines, or nanoparticles, of the present invention can be used to protect an individual against infection by heterologous EBV. That is, a vaccine made using an ENV protein from one Type of EBV is capable of protecting an individual against infection by a different Type of EBV. For example, a vaccine made using one or more ENV protein from Type I EBV, may be used to protect an individual against infection by a Type II EBV.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1

### Design of EBV Surface Protein-Based Nanoparticles

This Example describes the construction of nanoparticles comprising EBV surface proteins and ferritin or encapsulin.

Two potential platforms were considered for construction of self-assembling nanoparticles capable of displaying antigens on their surface: ferritin (Cho, et al. J Mol Biol, 2009; Stillman, et al. J Mol Biol, 2001) and encapsulin (Sutter, et al. Nat Struct Mol Biol, 2008). Ferritin forms a 4-3-2 point octahedron consisting of 24 subunits while encapsulin forms a 5-3-2 point icosahedron (T=1) consisting of 60 identical subunits. Comparison of ferritin structures revealed that several ferritins including human light chain (Z. Wang. C. Li, M. Ellenburg, E. Soistman, J. Ruble, B. Wright, J. X. Ho, D. C. Carter, Structure of human ferritin L chain. Acta Crystallogr D Biol Crystallogr 62, 800-806 (2006)) (PDB: 2ffx) and bullfrog lower subunit (J. Trikha, E. C. Theil, N. M. Allewell, High resolution crystal structures of amphibian red-cell L ferritin: potential roles for structural plasticity and solvation in function. J Mol Biol 248, 949-967 (1995)) (PDB: 1rcc) contain an N-terminal extension, which is not present in nonheme-type ferritins from Helicobacter pylori (K. J. Cho, H. J. Shin, J. H. Lee, K. J. Kim, S. S. Park, Y. Lee, C. Lee, S. S. Park, K. H. Kim, The crystal structure of ferritin from Helicobacter pylori reveals unusual conformational changes for iron uptake. J Mol Biol 390, 83-98 (2009)) (PDB: 3egm) or Escherichia coli (T. J. Stillman, P. D. Hempstead, P. J. Artymiuk, S. C. Andrews, A. J. Hudson, A. Treffry, J. R. Guest, P. M. Harrison, The high-resolution X-ray crystallographic structure of the ferritin (EcFtnA) of Escherichia coli: comparison with human H ferritin (HuHF) and the structures of the Fe (3+) and Zn(2+) derivatives. J Mol Biol 307, 587-603 (2001)) (PDB: leum). This N-terminal extension causes the most N-terminal residue to project radially from the assembled nanoparticle's center, and the termini to be evenly distributed on the surface of the ferritin particle. Thus, to test the idea that the N-terminal extension of bullfrog ferritin could be added to the bacterial counterpart to make its N-temini exposed and evenly distributed on the surface of the nanoparticle, a hybrid ferritin protein was constructed that combined sequence from H. pylori ferritin or E. coli ferritin with the N-terminal extension from bullfrog ferritin. The details of this construction are listed below.

Encapsulin has not been studied as a scaffold to present heterologous proteins on its surface. In contrast to ferritins, encapsulin has its C-termini exposed on the surface, thus providing a potential site at which to fuse exogeneous sequences. The C-termini of encapsulin are projected radially and are also located dispersedly around the 5-fold symmetry axes.

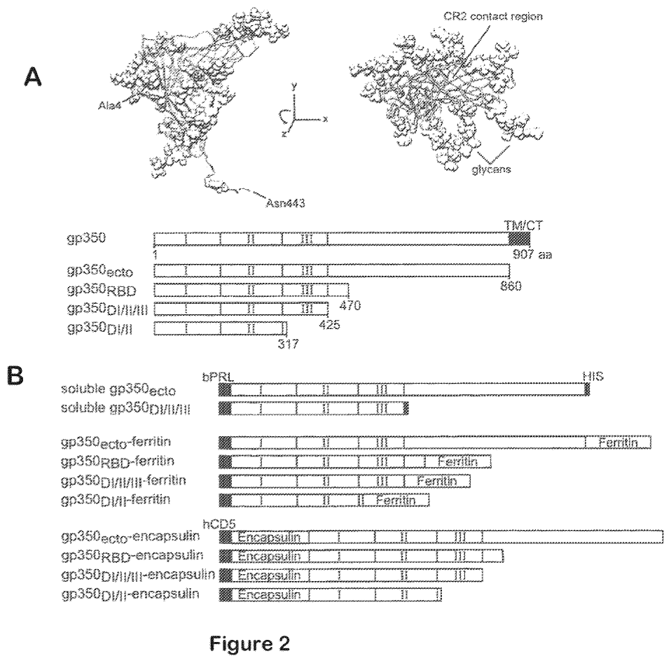

Glycoprotein 350 (gp350) of Epstein-Barr virus (EBV) is the most abundant viral surface protein and is a major target of neutralizing antibodies in naturally infected individuals (Thorley-Lawson DA, Poodry CA. Identification and isolation of the main component (gp350-gp220) of Epstein-Barr virus responsible for generating neutralizing antibodies in vivo. J Virol. 1982:43:730-736.). Gp350 is a type I transmembrane protein comprising of 907 amino acids containing an ectodomain of 860 amino acids, a transmembrane domain and a cytoplasmic tail. To test the ability of gp350 to induce an immune response, truncation variants of gp350 were fused to ferritin or encapsulin without disturbing the self-assembly capability of the self-assembling protein (see FIG. 2). These fusion proteins were constructed by creating expression vectors encoding the fusion genes encoding the gp350 variants and either H. pylori-bullfrog (HpBf), E. coli-bullfrog (EcBf) or encapsulin. The details of this construction are given below.

### A. Gene Synthesis and Vector Construction

All genes used in the study were optimized for mammalian codon usage. The gene encoding Helicobacter pylori-bullfrog hybrid ferritin was constructed by genetically fusing the N-terminus extension region (residues 2-9) of bullfrog (Rana catesbeiana) ferritin lower subunit (UniProtKB: P07797) to residues 3-167 of H. pylori nonheme ferritin (UniProtKB: Q9ZLI1. A point mutation was created at residue 8 (N8Q) of the bullfrog portion in order to abolish a potential N-linked glycosylation site. Similarly, point mutations were created at residue 7 (17E) and 19 (N19Q) of the H. pylori ferritin in order to make a salt bridge with 6R of bullfrog N-terminus part and abolish a potential N-linked glycosylation site, respectively.

The gene encoding Escherichia coli-bullfrog hybrid ferritin was constructed similarly but without the N8Q mutation in the bullfrog N-terminus extension region and with E. coli ferritin-1 (UniProtKB: POA998, residues 3-162 having a point mutation at residue 7 (17E) to make a salt bridge with 6R of bullfrog N-terminus part). These constructs also contained extra SG residues at the end of H. pylori or E. coli ferritin for cloning purpose.

The gene encoding encapsulin was constructed by genetically fusing the human CD5 signal sequence to Termotoga maritima bacteriocin (also known as maritimacin or encapsulin, UniProtKB: Q9WZP2, residues 1-264).

The gene encoding Epstein-Barr virus strain B95-8 full-length gp350 (UniProtKB: P03200, residues 1-907) was synthesized and the gene fragments corresponding to ectodomain (residues 2-860), receptor-binding domain (RBD, residues 2-470), domains I, II and III (D_{I/II/III} or D123, residues 2-425) and domains I and II (residues 2-317) were amplified by polymerase chain reactions with appropriate primers. Amplified gene fragments were genetically fused to a modified bovine prolactin (bPRL) secretion signal sequence and the hybrid ferritin with a SG linker to give rise to the gp350-ferritin fusion genes.

To construct the gp350-encapsulins fusion genes, amplified gene fragments were fused at the end of encapsulin gene with a (SG₃)₂ linker. To produce soluble gp350 ectodomain and D₁₂₃, the amplified gene fragments were fused with bPRL signal sequence and tagged with hexa-histidine at the end of the gp350 gene for purification purpose.

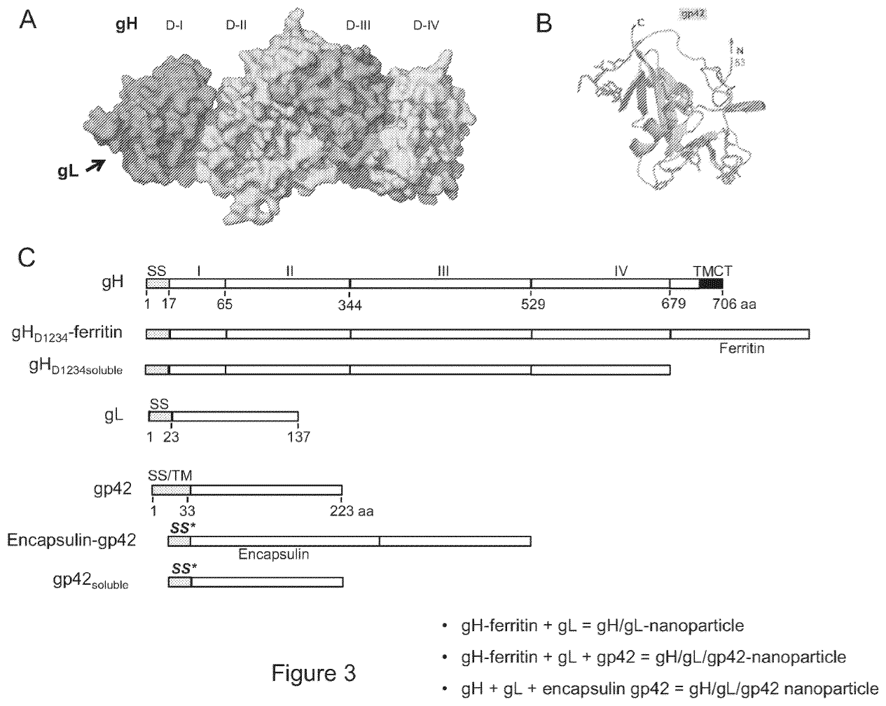

gH-ferritin was constructed by fusing the extracellular domain of gH (domains I, II, III, and IV) to the amino terminus of ferritin (FIG. 3).

Soluble gH was constructed by expressing the extracellular domain of gH (domains I, II, III, and IV) (FIG. 3).

Soluble gp42 was constructed by fusing a human CD5 leader (hCD5) sequence to the N-terminus truncated gp42 in place of gp42 amino acids 1-33.

The EBV gL protein used in this study is the fill-length, wild-type gL protein.

All gene constructs described above were cloned into CMV/R 8 κb (VRC 8405) mammalian expression vectors for efficient expression.

### B. Production and Purification of Recombinant Nanoparticles

FreeStyle 293-F or Expi293F cells (Life Technologies) were transiently transfected with the expression plasmids described in (A), either alone in or combination, using 293fectin or ExpiFectamine 293 transfection reagents, respectively (Life Technologies). For example, gp350 nanoparticles were made by transfecting individual gp350-ferritin or gp350-encapsulin constructs into recipient cells. gH/gL nanoparticles were made by co-transfecting plasmids expressing gH-ferritin and full length gL into cells, while gH/gL/gp42 nanoparticles were made by co-transfecting plasmids expressing gH-ferritin, full length gL, and soluble gp42 into cells. The cells were grown for 4 days after transfection, the culture supernatants harvested and the proteins or nanoparticles purified as described below.

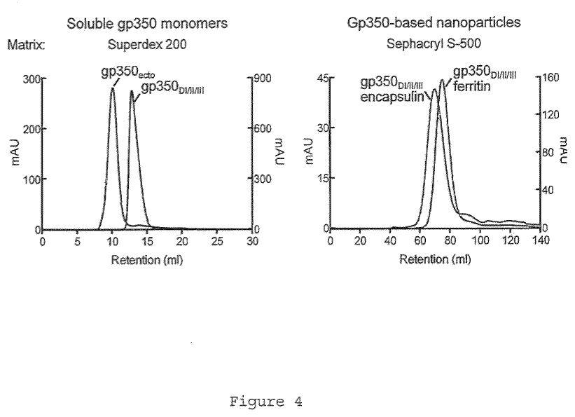

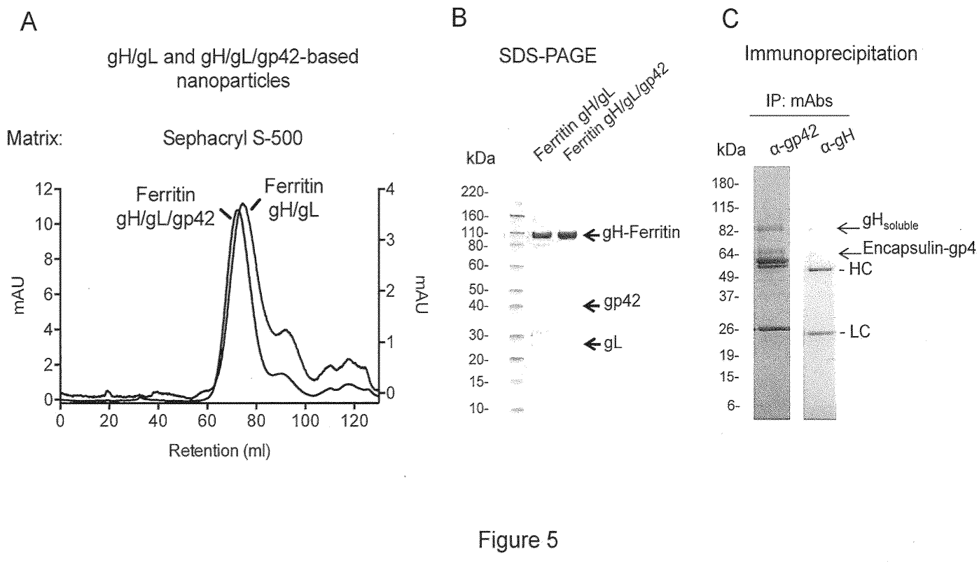

Nanoparticles were purified using affinity and size-exclusion chromatography. Briefly, the cleared cell culture supernatants were concentrated using a 30 kDa molecular weight cut-off ultrafiltration unit (Pall) after which the buffer was replaced with PBS. The nanoparticles were then applied to a Galanthus nivalis agglutinin (GNA, snowdrop lectin) affinity column (EY Laboratories) and eluted using a solution of 1.0 M methyl α-D-mannopyranoside in PBS. The nanoparticles were further purified by size exclusion column chromatography using a HiPrep 16/60 Sephacryl S-500 HR column (GE Healthcare Life Sciences) in PBS and the peak fraction collected and used for further studies. The SEC chromatogram for the gp350-based nanoparticles is shown in FIG. 4 (right) while a SEC chromatogram for gH/gL-ferritin and gH/gL/gp42-ferritin nanoparticles is shown in FIG. 5A

Soluble proteins were purified using ion affinity and SEC chromatography. Briefly, cleared cell culture supernatants were adjusted to 50 mM Tris, pH 8 and 500 mM NaCl, and applied to a metal ion affinity chromatography column containing Ni sepharose excel resin (GE Healthcare Life Sciences). After washing the column with buffer (50 mM Tris, 500 mM NaCl, 30 mM imidazole, pH 8.0), the proteins were eluted using elution buffer (50 mM Tris, 500 mM NaCl, 300 mM imidazole, pH 8.0). The proteins were then further purified by size exclusion column chromatography using a Superdex 200 10/300 GL or a Superose 6 10/300 GL column (GE Healthcare Life Sciences) in PBS and the peak fraction collected and used for further studies. A SEC chromatogram for soluble gp350 monomers is shown in FIG. 4 (left).

### C. Characterization of EBV gp350-based, gH/gL-Based, and gH/gL/gp42-Based Nanoparticles

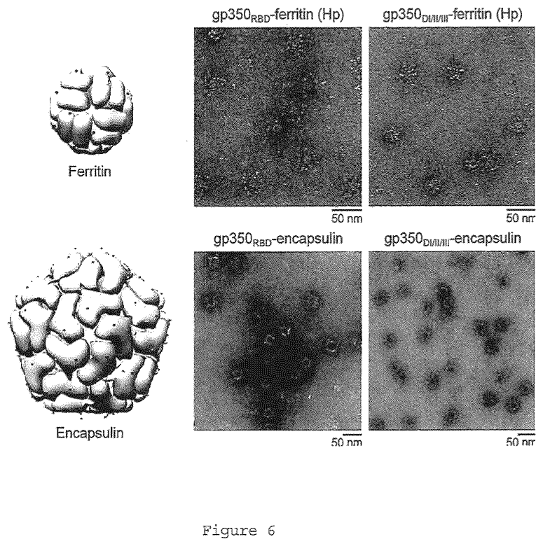

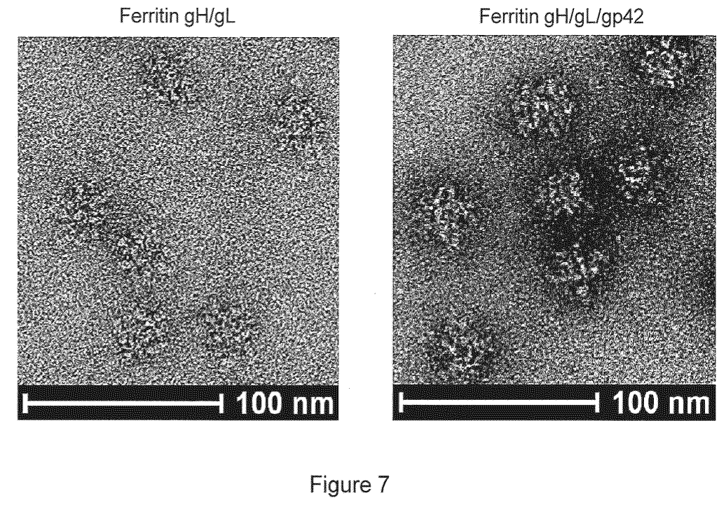

The purified nanoparticles were further examined by electron microscopic (EM) analysis. Briefly, for negative staining EM analysis, samples of about 50 μg ml⁻¹ were adsorbed to freshly glow-discharged carbon-coated grids, rinsed with PBS, and stained with 2% ammonium molybdate or 0.75% uranyl formate. Images were recorded on an FEI T20 microscope with a Eagle CCD camera. EM images of gp350-ferritin and gp350-encapsulin nanoparticles are shown in FIG. 6 while gH/gL ferritin-based nanoparticles and gH/gLgp42 ferritin-based nanoparticles are shown in FIG. 7, left and right, respectively. This analysis confirmed that the expressed proteins formed nanoparticles having globular protrusions from the spherical nanoparticle core.

### Example 2

Antigenicity of gp350-Based Nanoparticles

To verify the antigenicity of the gp350-based nanoparticle, the reactivity of the nanoparticles was tested using the anti-gp350 monoclonal antibodies (mAbs) MAb 72A1 and MAb 2L10. MAb 72A1 recognizes the receptor-binding site of gp350, which mediates viral attachment to the host cell receptor, complement receptor 2 (CR2 or CD21). MAb 72A1 also potently neutralizes EBV (G. J. Hoffman, S. G. Lazarowitz, S. D. Hayward, Monoclonal antibody against a 250,000-dalton glycoprotein of Epstein-Barr virus identifies a membrane antigen and a neutralizing antigen. Proc Natl Acad Sci U S A 77, 2979-2983 (1980), Sairenji T, Bertoni G, Medveczky MM, Medveczky PG, Nguyen QV, Humphreys RE., Inhibition of Epstein-Barr virus (EBV) release from P3HR-1 and B95-8 cell lines by monoclonal antibodies to EBV membrane antigen gp350/220. J Virol. 1988 August; 62 (8): 2614-21.). The CR2-binding site (CR2BS) is one of the sites of vulnerability on the virus and therefore an attractive target for vaccine development. MAb 2L10 is a non-neutralizing antibody and does not compete with 72Al (J. Luka, R. C. Chase, G. R. Pearson, A sensitive enzyme-linked immunosorbent assay (ELISA) against the major EBV-associated antigens. I. Correlation between ELISA and immunofluorescence titers using purified antigens. J Immunol Methods 67, 145-156 (1984)). To test the reactivity of the expressed proteins and nanoparticles with these antibodies, 100 ul of purified nanoparticles or soluble gp350 protein (at 25 nM) were coated onto MaxiSorp plates (Nunc). To each well was then added anti-gp350 antibody (MAb 72Al or MAb 2L10) or anti-influenza hemagglutinin (MAb C179). Following appropriate incubation and removal of the antibody, bound MAbs were detected using a peroxidase-conjugated, secondary antibody (anti-mouse IgG) (Southern Biotech).

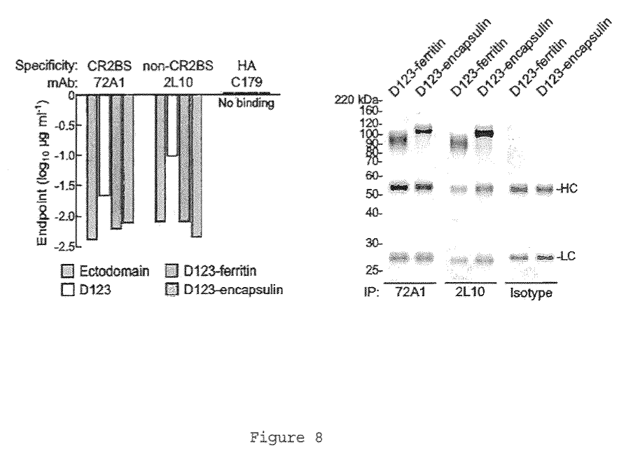

The results of this study, which are shown in FIG. 8 (left), showed that both 72A1 and 2L10 mAbs recognized gp350-based nanoparticles as well as soluble gp350 ectodomain. In addition, both antibodies bound the soluble D 12: monomer to a lesser extent. Neither of the purified proteins bound the isotype control, MAb C179.

The antigenicity of the gp350-based nanoparticles was further confirmed by immunoprecipitation. Briefly, five micrograms of mAbs directed to gp350 CR2-binding site (72A1), gp350 non-CR2-binding site (2L10) or influenza hemagglutinin (C179) were incubated with purified nanoparticles (5 μg) at room temperature for 30 minutes. After incubation, pre-washed protein G Dynabeads (Life Technologies) were added to the reactions and incubated for another 30 minutes. PBS containing 0.01% Tween 20 was used as washing buffer. Immune complexes were then magnetically separated, washed and eluted in Lamini buffer containing reducing agent. A half volume of the reactions were then analyzed by SDS-PAGE. The results of this analysis are shown in FIG. 8 (right).

### Example 3

Immune Response Induced by gp350-Based Nanoparticles

To evaluate the immunogenicity of the gp350-based nanoparticles, ten-week old mice were immunized at weeks 0 and 3 with 5.0 μg of both HpBf and EcBf ferritins and encapsulin nanoparticles expressing either gp350 RBD or D₁₂₃ on the surface in the presence of Sigma Adjuvant System (SAS, also known as Ribi). Immune sera were collected at 2 weeks after each immunization to analyze antibody response to gp350. Briefly, soluble gp350 ectodomain protein (2 μg ml⁻¹, 100 μl well⁻¹) were coated onto wells of a MaxiSorp plate (Nunc), after which to each well was added an aliquot of serially diluted immune sera. Following incubation and removal of the immune sera, to each well was added peroxidase-conjugated secondary antibody (anti-mouse IgG). Following incubation and removal of unbound secondary antibody, the wells were developed using a SureBlue chromogen (KPL) and the reaction was stopped by adding 0.5 M sulfuric acid. Absorbance at 450 nm was then measured by SpectraMax M2e (Molecular Devices). Endpoint titers were determined by calculating concentrations at the absorbance threshold (four times background) from the binding curve.

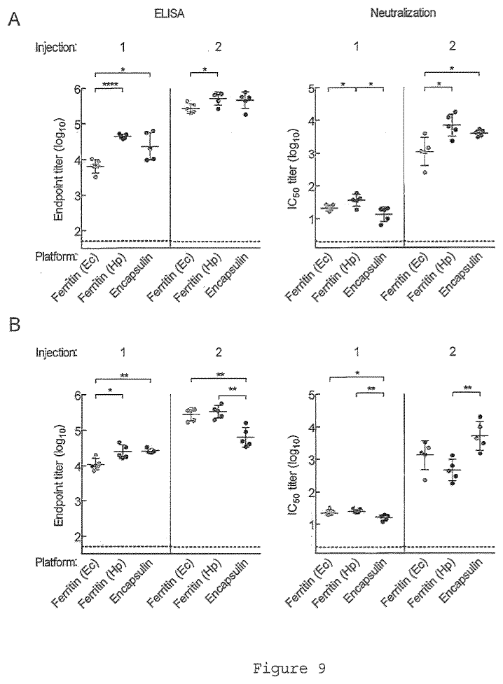

The results of this study, which are shown in FIG. 9, demonstrate that an antibody response to gp350 was detected in all gp350-based nanoparticle-immunized mice after a single dose at titers of ˜10^{3.9}-10^{4.9} and the titers were boosted about 10-fold by a second dose. Neutralizing antibody titers were also detected in all mice after a single dose although the titers were not high (IC₅₀<50). These titers were markedly boosted at ˜100-fold by a second dose.

### Example 4

Comparison of gp350-Based Nanoparticles and Soluble gp350 Protein

To compare immune responses elicited by the gp350-based nanoparticles to that of soluble gp350 proteins, ten-week old mice were immunized at weeks 0 and 3 with either soluble gp350 ectodomain or D₁₂₃ or D₁₂₃-nanoparticles at 5.0 and 0.5 μg in the presence of SAS as adjuvant. Immune sera were collected 2 weeks after the first (1) and the second (2) immunization and analyzed as described in Example 2 and 3.

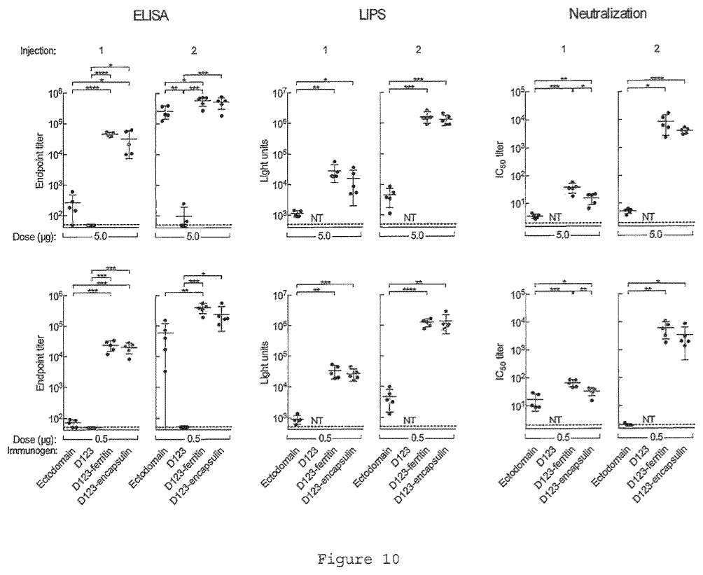

The results of this study, which are shown in FIG. 10, show that at 2 weeks following the first immunization the D₁₂₃ nanoparticles elicited a higher antibody response than did either the soluble gp350 ectodomain or soluble D₁₂₃ protein (titers of 47,654±16,482 and 32,042±24907 for D₁₂₃-ferritin and D₁₂₃-encapsulin, respectively vs. 261±219 and <50 for gp350 ectodomain and D₁₂₃, respectively). However, following a second immunization, soluble gp350 ectodomain boosted ELISA titers dramatically to the titers at only ˜2-3-fold lower than that of either D₁₂₃-ferritin-or D₁₂₃-encapsulin-immunized groups (261,116±116,301 vs. 567,764±188,536 or 499,128±211,748, respectively). Surprisingly, soluble gp350 D₁₂₃ failed to elicit antibody responses even after the second immunization (titers of 96±92) although the same D₁₂₃ displayed on ferritin and encapsulin was highly immunogenic. Because an earlier study (Sashihara, et al. Virology, 2009) demonstrated a strong correlation between titers measured in neutralization assay and immunoprecipitation-based assay (luciferase immunoprecipitation system, LIPS), the mouse immune sera was tested in an LIPS assay in addition to ELISA. For the LIPS assay, the fusion protein composed of gp350 and Remilla luciferase was incubated with sera and immunoprecipitated using protein A/G beads (Thermo Scientific) in 96-well filter bottom plates (Millipore). Luciferase activity of antibody-bound fusion proteins was then measured by adding coelenterazine substrate (Promega) and detecting by Centro LB 960 luminometer (Berthold Technologies).

These studies demonstrate that LIPS antibody titers in the sera of gp350 ectodomain-immunized mice were more than two logs lower than that of either D₁₂₃-ferritin-or D₁₂₃-encapsulin-nanoparticle-immunized mice (10^{3.6±0.3} vs. 10^{6.2±0.2} or 10^{6.1±0.2}, respectively) at 2 weeks after a second dose. To verify if the LIPS titers reflected neutralizing antibody titers, serum neutralizing antibody titers were determined by GFP-reporter assay (Sashihara, et al. Virology, 2009). Neutralization of EBV to B cells has been described previously (Sashihara, J., Burbelo, P. D., Savoldo, B., Pierson, T. C., Cohen, J. I., Human antibody titers to Epstein-Barr virus (EBV) gp350 correlate with neutralization of infectivity better than antibody titers to EBV gp42 using a rapid flow cytometry-based EBV neutralization assay. Virology 391, 249-256 (2009).) Briefly, immune sera were serially diluter in a 2-fold step and 25 ul of the diluted samples was incubated with B95-8/F virus for 2 hours. The mixture was added to Raji cells in the 96-well plate and incubated for 3days at 37° C. (Raji cells were propagated in RPMI 1640 with complete supplements: 10% fetal bovine serum, 100 U/ml penicillin, 100 ug/ml streptomycin and 2 mM L-glutamine) Following incubation, cells were fixed in 2% paraformaldehyde in PBS and analyzed by Accuri C6 flow cytometer and BD CSampler software (BD Biosciences, San Jose, CA, USA) to quantify the percentage of infected ells based on GFP expression. Neutralization antibody titers were expressed as the concentration of serum antibody needed to inhibit viral entry by 50% (IC50) calculating with controls in the absence of virus (0% infection) or serum (100% infection).

Surprisingly, the differences in neutralizing antibody titers between the sera from soluble gp350 ectodomain-immunized mice and that from either D₁₂₃-ferritin-or D₁₂₃-encapsulin-immunized mice were ˜1000-fold after a second dose (5±1 vs. 8,594±5944 or 3,939±874, respectively) and the titers in soluble gp350 ectodomain-immunized group were barely above the threshold of the assay (IC₅₀<10). Together, the results revealed that immunization using soluble gp350 ectodomain induced mostly non-neutralizing antibodies which were only detectable in ELISA. ELISA, LIPS and neutralizing antibody titers in mice immunized with 5.0 and 0.5 μg of D₁₂₃-ferritin and D₁₂₃-encapsulin were virtually the same (<2-fold differences), while there was a slight reduction in ELISA and neutralizing antibody titers in mice immunized with both soluble gp350 ectodomain (4.4-fold decrease in ELISA titers and no neutralizing antibody titers in group immunized with lower dose).

### Example 5

Durability of Neutralizing Antibody Response in gp350-Based Nanoparticle-Immunized Animals

To assess the kinetics of virus neutralizing antibody responses in the mice immunized in Example 4, the titers of neutralizing antibody were determined longitudinally for >6 months. At 2 months following the second immunization, the neutralizing titers in D₁₂₃-ferritin- and D₁₂₃-encapsulin-immunized animals at both 5.0 and 0.5 μg doses declined about one log to 10^{2.4}-10^{3.1}, however, the titers remained at the same level for another month without further immunization. All mice then received a third dose at week 16 and their serum neutralizing antibody titers monitored.

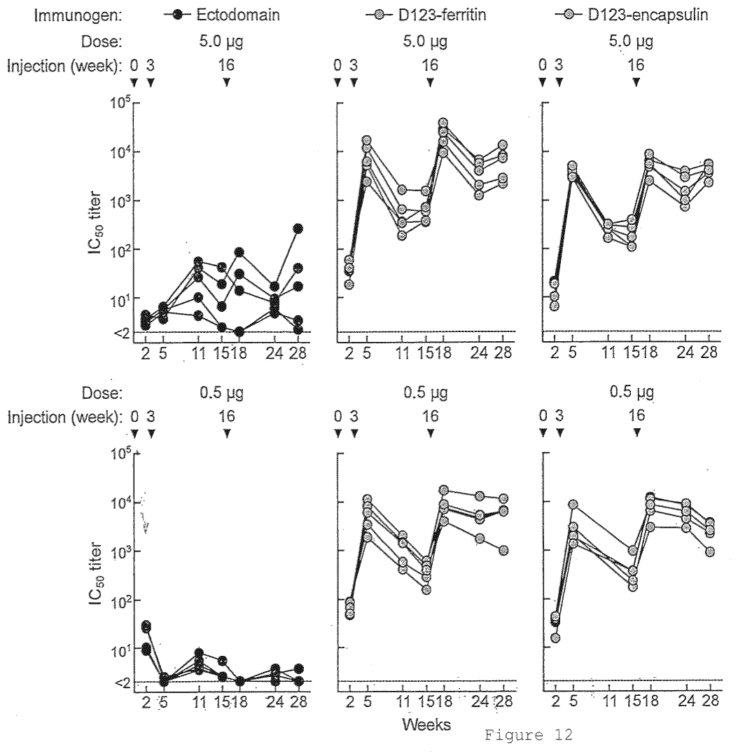

The results of this study are shown in FIG. 12. Expectedly, the titers were boosted by 33- and 26-fold in D₁₂₃-ferritin and D₁₂₃-encapsulin, respectively (10^{4.3±0.2} and 10^{3.7±0.2}, respectively), whereas the effect was not obvious in groups immunized with soluble gp350 ectodomain as measured at 2 weeks after the boost (10^{1.0±0.7}). The peak neutralizing antibody titers in D₁₂₃-ferritin were higher than that after the second immunization (10^{3.8±0.3}) and the titers did not wane as quickly as after the second immunization. The titers at 2 and 3 months after the third immunization were stable at 10^{3.3}-10^{3.8} in groups immunized with D₁₂₃-ferritin and D₁₂₃-encapsulin and that were 1.5-5.9 times lower than the peak and one log higher than the titers at the same time point after the second immunization. Similar kinetics and magnitudes of neutralizing antibody titers were observed when animals were immunized with a 10× smaller dosage (0.5 μg) of either gp350-based nanoparticles. However, at this dose the soluble gp350 ectodomain was unable to elicit any neutralizing antibody response.

### Example 6

Characterization of Antibodies Elicited by gp350-Based Nanoparticles

To determine the fine specificity of the antibodies elicited by either soluble gp350 ectodomain or gp350-based nanoparticles, a surface plasmon resonance (SPR)-based antibody competition assay was performed. This assay detects specific populations of antibodies in immune sera, the specificity of which is similar to that of a competing mAb. Briefly, the soluble gp350 ectodomain was immobilized on a sensor chip via amine coupling reaction. Before measuring the binding of the immune sera, 72A1, 2L10 or C179 was injected to the flow cell to saturate the sites where the mAb recognizes on the chip. Immune sera (taken 2 weeks after the second immunization with Ribi-adjuvanted 5 μg of immunogen) was then injected to the antibody-saturated flow cells and the binding kinetics of the serum antibodies were measured. The serum antibodies directed to the same epitope as 72A1 were not able to bind to the 72A1-saturated gp350 and therefore resulted in lower overall binding compared to that of C179-saturated gp350. All data were normalized with an isotype control (C179-saturated) and shown as fraction response. Inhibition of serum antibody to bind to gp350 by 72A1 or 2L10 (X) was calculated by an equation: X=100−{(maximum response unit of 72A1- or 2L10-saturated flow cell/maximum response unit of isotype antibody-saturated flow cell)×100}. Surprisingly, when the competitor antibody was CR2BS-directed mAb 72A1, 52±11 and 60±12% of total anti-gp350 antibodies in the immune sera elicited by D₁₂₃-ferritin and D₁₂₃-encapsulin, respectively, was competed, whereas only 5±4% of anti-gp350 antibodies in immune sera elicited by soluble gp350 ectodomain was blocked.

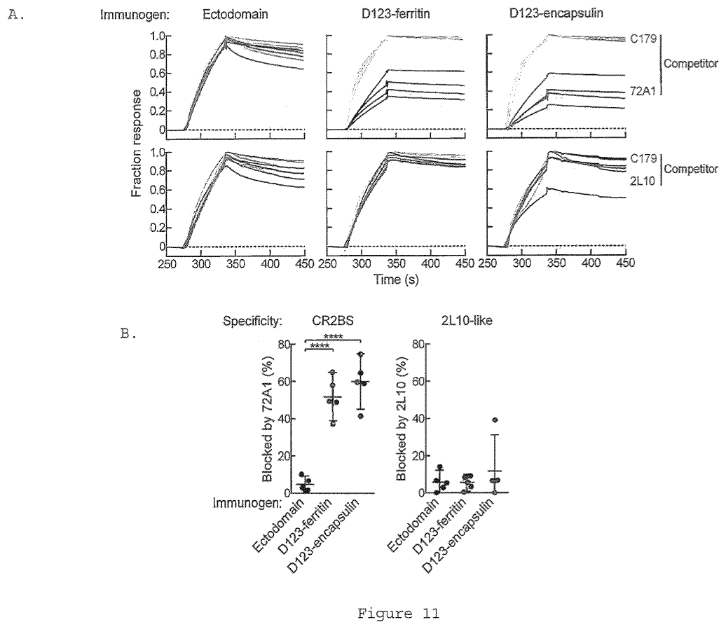

The results of this analysis, which are shown in FIG. 11, demonstrate that the CR2BS on the gp350-based nanoparticles were indeed predominantly targeted by the antibody response and the same site on soluble gp350 ectodomain was not. Importantly, there was negligible fraction of anti-gp350 antibodies in these immune sera that was competed by a non-neutralizing, non-CR2BS-directed mAb 2L10 (6±5, 5±4 and 12±16% in soluble gp350 ectodomain, D₁₂₃-ferritin and D₁₂₃-encapsulin, respectively), suggesting the epitope recognized by 2L10 was not immunodominant in both soluble gp350 ectodomain and gp350-based nanoparticles.

### Example 7

Comparison of Serum Neutralization Titers in EBV-Positive Human Individuals and gp350-Based Nanoparticle-Immunized Mice

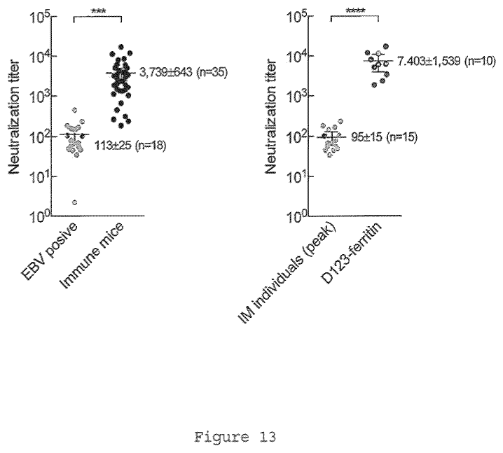

Sera from mice immunized in Examples 3 and 4 were obtained and serum neutralization titers determined. These titers were then compared to neutralization titers observed in humans naturally infected with EBV. FIG. 13. (left) shows a comparison of serum neutralization titer (IC₅₀) between a combination of EBV-seropositive individuals and persons with EBV-positive mononucleosis and gp350-based nanoparticle-immunized mice; (right) Comparison of serum neutralization titer (IC₅₀) between persons with EBV-positive infectious mononucleosis and D123-ferritin-immunized mice.

### Example 8

Antigenicity of gH/gL and gH/gL/gp42-Based Nanoparticles

The ability of purified nanoparticles to induce antibody production was tested in mice. Briefly, ten week old BALB/c mice (n=5) were injected intramuscularly with 0.5 ug of either soluble gH/gL, soluble gH/gL/gp42, ferritin-gH/gL or ferritin-gH/gL/gp42. A second round of injections was given three weeks later. At 5 weeks post-first injection, blood was drawn and antibody titers to gH, gL and gp42 were determined using a LIPS (luciferase immunoprecipitation assay) described by Sashihara, et al., Virology 391, 249-256, 2009.

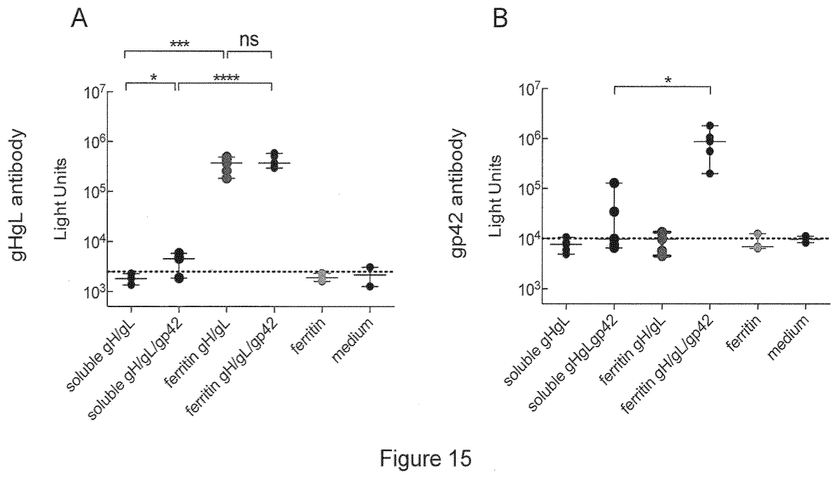

The result of this analysis, which is shown in FIG. 15, shows that ferritin-based gH/gL nanoparticles or ferritin-based gH/gL/gp42 nanoparticles induce levels of antibody to the gH/gL complex that are significantly higher than the levels induced by soluble gH or gL protein. Additionally, ferritin-based gH/gL/gp42 nanoparticles induce levels of antibodies to gp42 that are significantly higher than the levels induced by soluble gH/gL/gp42.

### Example 9

### Comparison of Neutralizing Antibody Induction

The ability of purified nanoparticles to induce neutralizing antibodies in mice was measured using a B-cell and epithelial cell neutralization assay. Briefly, ten week old BALB/c mice (n=5) were immunized as described in Example 8. At 2 weeks post-second injection, blood was drawn and the ability of the sera to inhibit infection of B-cells and epithelial cells by a recombinant reporter virus expressing GFP (Sashihara J., et al., Virology, 391, 249-256, 2009) was tested.

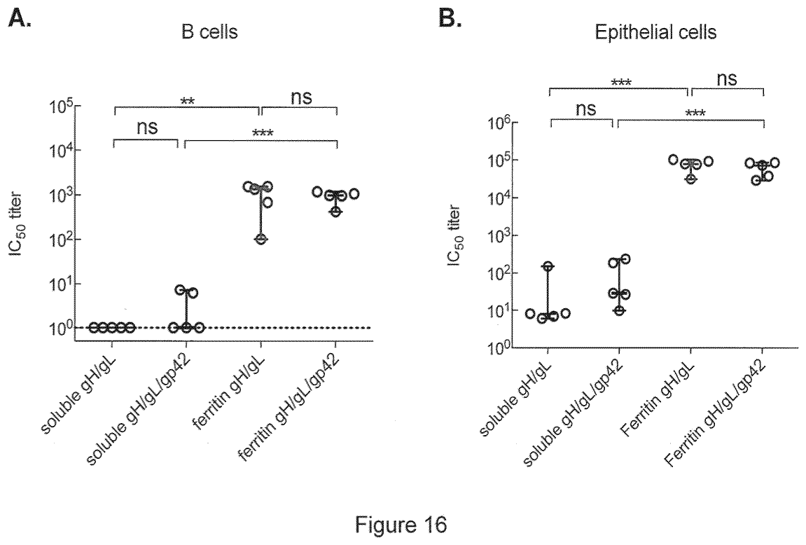

The results of this analysis, which is shown in FIG. 16, shows that ferritin-based gH/gL nanoparticles or ferritin-based gH/gL/gp42 nanoparticles induce levels of neutralizing antibodies to EBV that are significantly higher than the levels induced by soluble gH/gL protein or soluble gH/gL/gp42 protein.

Example 10

Comparison of Kinetics of gH/gL Antibody Titers in Sera From Mice Immunized With 0.5 μg soluble gH/gL, gH/gL Nanoparticles, Soluble gH/gL/gp42, or gH/gL/gp42 Nanoparticles

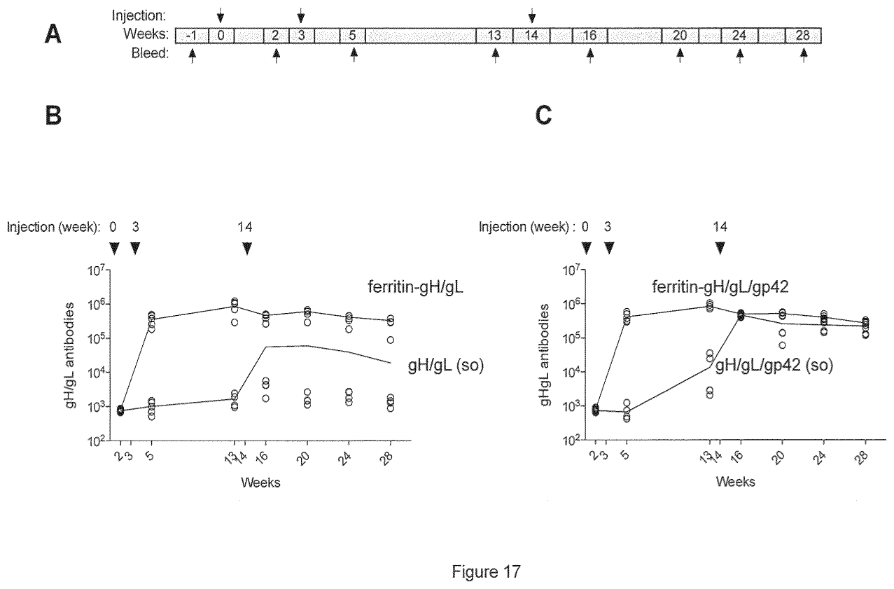

Ferritin-based nanoparticles and soluble proteins were prepared as described in Example 1. Balb/c mice (n=5) were then injected intramuscularly with 0.5 ug soluble gH/gL, soluble gH/gL/gp42, ferritin-gH/gL or ferritin-gH/gL/gp42 at weeks 0, 3 and 14. Blood was drawn at weeks 2, 5, 13, 16, 20, 24 and 28, and antibody titers to gH/gL were determined using a LIPS assay, as described by Sashihara, et al., Virology 391, 249-256, 2009. The results of this study, which are shown in FIG. 17, demonstrate that gH/gL antibody responses with gH/gL-nanoparticles are higher than soluble gH/gL protein and the antibody titers are sustained >12 weeks after the 3^{rd} dose. The results also demonstrate that gH/gL antibody titers in mice immunized with gH/gL/gp42-nanoparticles are higher than soluble gH/gL/gp42 after 2 doses, but comparable to soluble gH/gL/gp42 after 3 doses of vaccine. Finally, the result show that antibody titers are sustained >12 weeks after the 3^{rd dose.}

### Example 11

Comparison of Kinetics of gp42 Antibody Titers in Sera From Mice Immunized With Soluble gH/gL/42 or gH/gL/gp42 Nanoparticles

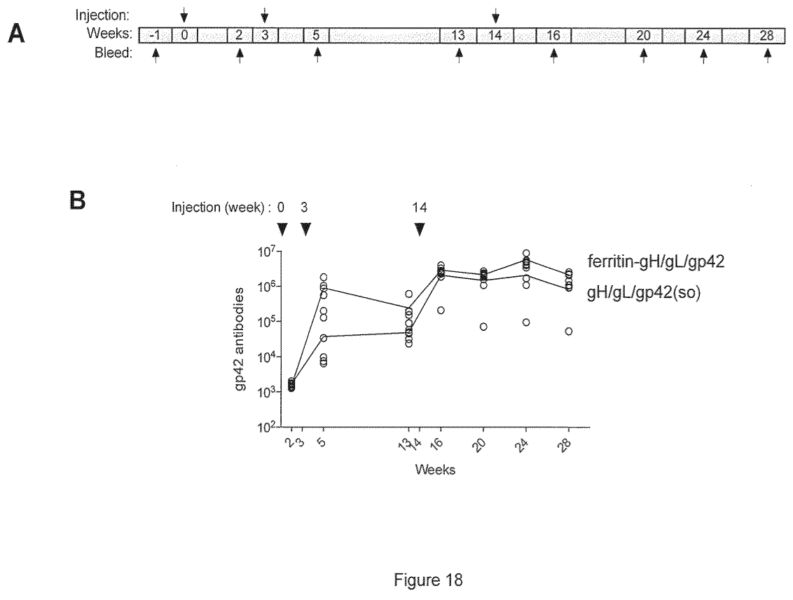

The production of nanoparticles and soluble proteins, and the immunization and blood draws were performed as described in Example 10. Antibody titers to gp42 were then determined by LIPS assay, as described by Sashihara, et al., Virology 391, 249-256, 2009. The results of this study, which are shown in FIG. 18, demonstrate that gp42 antibody titers in mice immunized with gH/gL/gp42-nanoparticles are higher than soluble gH/gL/gp42 after 2 doses, but comparable to soluble gH/gL/gp42 after 3 doses of vaccines. The results also show that gp42 antibody titers are sustained >12 weeks after the 3^{rd dose.}

Example 12

Comparison of Kinetics of B Cell Neutralizing Antibody Titers in Mice Immunized With Soluble gH/gL, gH/L Nanoparticles, Soluble gH/gL/gp42, or gH/gL/gp42Nanoparticles

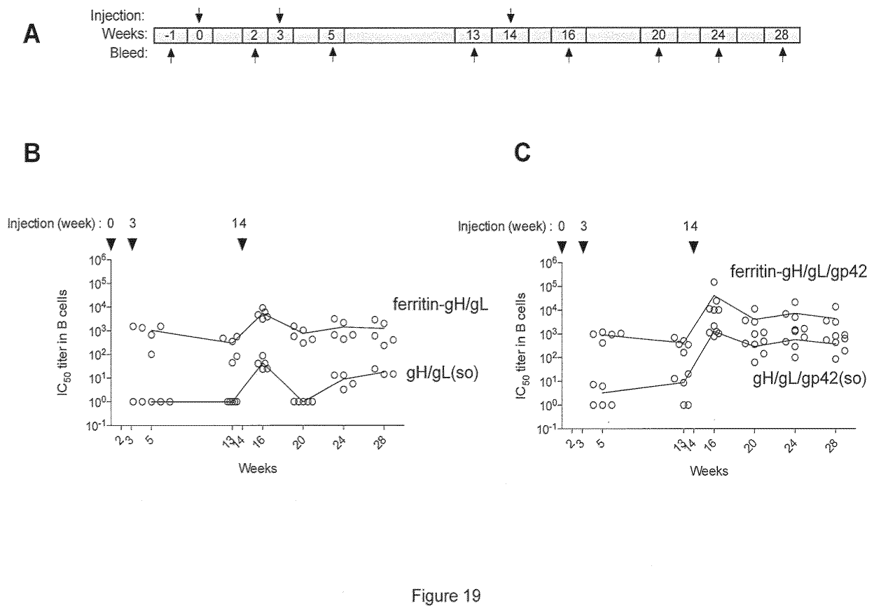

The production of nanoparticles and soluble proteins, and the immunization and blood draws were performed as described in Example 10. The ability of the mouse sera to neutralize EBV infection of B cells was then tested, as described in Example 9. The results of this study, whoch are shown in FIG. 19, demonstrate that B cell neutralizing antibody responses with gH/gL-nanoparticles or gH/gL/gp42-nanoparticles are higher than soluble gH/gL or gH/gL/gp42 proteins, respectively. The results also demonstrate that B cell neutralizing antibody titers are sustained >12 weeks after the 3rd dose.

### Example 13

Comparison of Kinetics of Epithelial Cell Neutralizing Antibody Titers in Mice Immunized With Soluble gH/gL, gH/gL Nanoparticles, Soluble gH/gL/gp42, or gH/gL/gp42 Nanoparticles

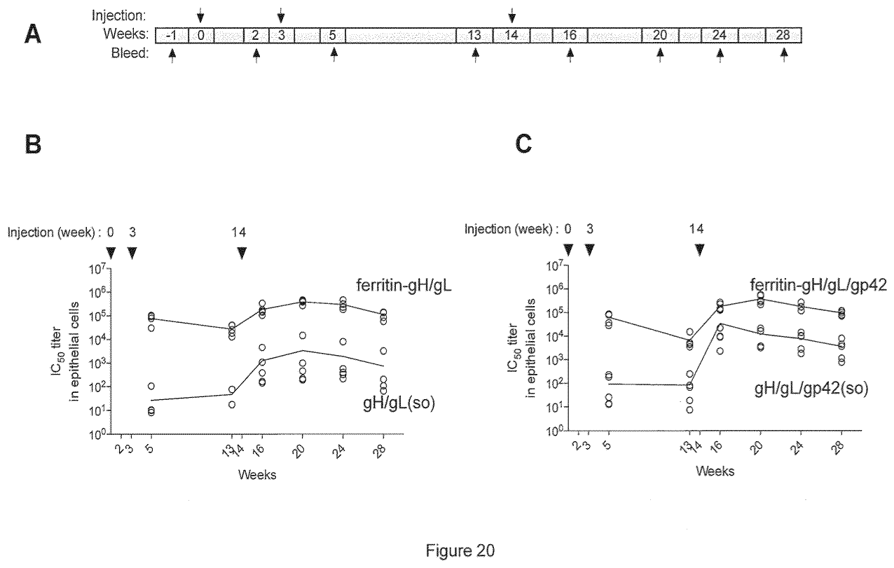

The production of nanoparticles and soluble proteins, and the immunization and blood draws were performed as described in Example 10. Epithelial cell neutralization assay were performed as described in Example 9. The results of this study, which are shown in FIG. 20, demonstrate that epithelial cell neutralizing antibody responses with gH/gL-nanoparticles or gH/gL/gp42-nanoparticles are higher than soluble gH/gL or gH/gL/gp42 proteins, respectively. The results also show that epithelial cell neutralizing antibody titers are sustained >12 weeks after the 3rd dose.

### Example 14

B cell and Epithelial Cell Neutralizing Antibody Titers After the 3^{rd} Dose in Sera of Mice Immunized With Soluble Proteins or Nanoparticles Compared to Sera From Naturally Infected Humans

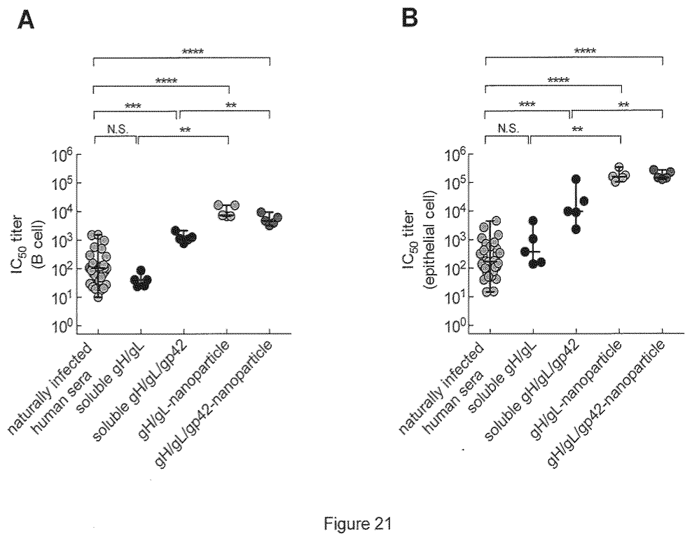

The B cell neutralizing antibody titers obtained in Example 12 and the epithelial cell neutralizing antibody titers obtained in Example 13 were compared to neutralization titers observed in human sera from individuals naturally infected with EBV. FIG. 21 shows B cell neutralizing antibody titers (left) or epithelial cell neutralizing titers (right) in human sera compared to sera from mice immunized with soluble proteins or nanoparticles.

This comparison shoes that B cell neutralizing antibody titers in mice immunized with nanoparticles are >20-fold higher than that in naturally infected humans, and that epithelial cell neutralizing antibody titers in mice immunized with nanoparticles are >100-fold higher than that in naturally infected humans.

### Example 15

Generation of gH/gL-Nanoparticles or gH/gL/gp42-Nanoparticles From Single Polypeptides

To facilitate manufacture of dimeric gH/gL-nanoparticle and trimeric gH/gL/gp42-nanoparticles for clinical studies, constructs were made that express a polyprotein capable of forming a nanoparticle containing multiple EBV proteins. Specifically, constructs were made that express either a ferritin-gH/gL polypeptide or a ferritin-gH/gL/gp42 polypeptide. Each of these polypeptides was designed to include furin and picornavirus 2A cleavage sites and so that they produce ferritin-based nanoparticles by self-cleavage. The polypeptides also comprise a leader peptide sequence from human CD5 protein, in order o facilitate secretion of the polyprotein from the cell. FIG. 22A illustrates the structure of two different polyproteins.

Nanoparticles were produced as described in Example 1. Briefly, proteins were purified from supernatant of cells co-transfected with multiple plasmids expressing individual proteins or cells transfected with plasmids expressing the gH/gL or gH/gL/gp42 polypeptide. Purification of nanoparticles by size exclusion chromatography was then performed as described in Example 1A. SDS-PAGE analysis of the purified gH/gL nanoparticles and gH/gL/gp42 nanoparticles (FIG. 22 (B) shows that gH/gL-nanoparticles and gH/gL/gp42-nanoparticles can be produced by transient transfection of single plasmids that express a single polyprotein that is processed by self-cleavage.

### Example 16

Immunogenicity of gp350 Nanoparticles in Non-Human Primates

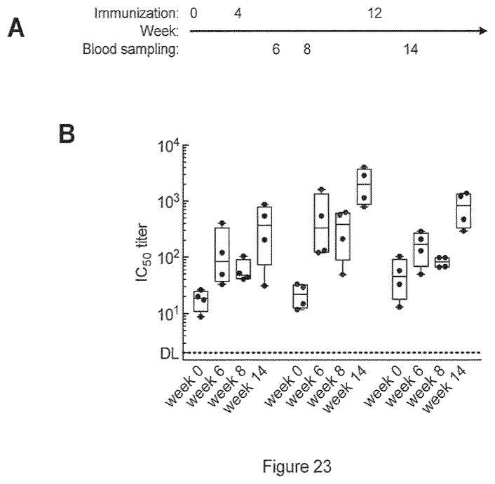

To evaluate the ability of purified nanoparticles to induce neutralizing antibodies in a species closer to humans than mice, cynomolgus macaques (Macaca fascicularis) were immunized with gp350 nanoparticles. Briefly, twelve monkeys were divided into three groups and given 50 μg of gp350 ectodomain, or 25 μg of either gp350 D₁₂₃-ferritin or gp350 D₁₂₃-encapsulin with adjuvant (Sigma Adjuvant System) on weeks 0, 4 and 12. Blood was drawn prior to immunization and at weeks 6, 8 and 14, and neutralizing antibody titer determined. The resutls are shown in FIG. 23.

Cross reacting EBV neutralizing antibody was found in all of the monkeys prior to immunization (IC₅₀ titers from 10^{1.1} to 10^{2.0}), which is unsurprising since cynomolgus monkeys are naturally infected with a lymphocryptovirus that shares homology with EBV. EBV neutralizing antibody titers were increased after two immunizations (week 6) in all groups and the titers were further boosted by a third dose (week 14). Neutralizing antibody titers in monkeys immunized with gp350 D₁₂₃-ferritin (center four bars) and D₁₂₃-encapsulin (right four bars) were 10^{3.3±0.3} and 10^{2.8±0.3}, respectively, and were higher than that of soluble gp350-immunized monkeys (10^{2.4±0.6}) (left four bars). These results demonstrate the immunogenicity of gp350-based nanoparticles in a second species of animal.

### Example 17

Protective Immunity Against Experimental Infection of Mice With Recombinant Vaccinia Virus Expressing EBV gp350

The ability of gp350 nanoparticle vaccines to protect mice from challenge with recombinant vaccinia virus expressing EBV gp350 was assessed by immunizing mice as described in Examples 4 and 5. Ten months after the final immunization, the mice were challenged with 10⁶ pfu of recombinant vaccinia virus expressing EBV gp350 by the intranasal route. Body weights and clinical symptoms were monitored daily.

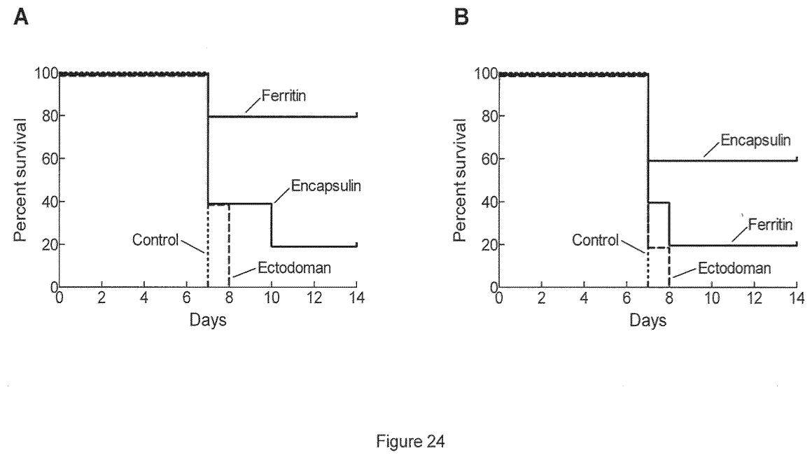

The results of this study, which are shown in FIG. 24, demonstrate that mice immunized with gp350 D₁₂₃-ferritin and gp350 D₁₂₃-encapsulin were partially protected (up to 80% survival) against lethal challenge with vaccinia virus expressing gp350. In contrast, all non-immunized control mice and animals immunized with soluble gp350 ectodomain, with the exception of one, died as a result of the challenge virus infection. These results demonstrate that immunization with gp350-based nanoparticles provides partially protective immunity against challenge with recombinant vaccinia virus expressing gp350.

### Example 18

Immunogenicity of gp350 Nanoparticles in Aluminum Phosphate Gel Adjuvant

The ability of purified nanoparticles to induce neutralizing antibodies in aluminum phosphate gel (alum) adjuvant, which is approved for use in humans, was tested in mice. Mice were divided into 6 groups; 3 groups received 5 μg of gp350 D₁₂₃-ferritin and 3 groups received D₁₂₃-encapsulin. The vaccines were given with no adjuvant, aluminum phosphate gel (alum) adjuvant, or Sigma Adjuvant System (SAS) at weeks 0, 4 and 16.

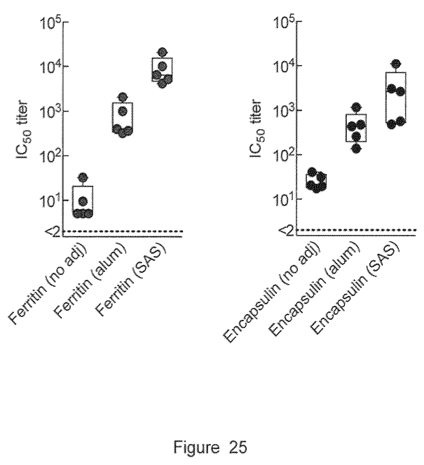

The results of this study, which are shown in FIG. 25, demonstrate that EBV neutralizing antibodies titers were higher in mice immunized with gp350 nanoparticles with adjuvant than in animals immunized with gp350 nanoparticles but without adjuvant. Animals that received gp350 D₁₂₃-ferritin (left) or gp350 D₁₂₃-encapsulin (right) vaccines in SAS adjuvant had about 10-time higher titers of EBV neutralizing antibody than animals that received the same vaccines in alum adjuvant. These results demonstrate that gp350 nanoparticle vaccine in alum adjuvant is capable of inducing EBV neutralizing antibody titers of 10^{2.6±0.3} to 10^{2.8±0.3}, which are 5-8 times higher than the EBV neutralizing titers in naturally infected humans.

1-12. (canceled).

13. A recombinant protein comprising at least 25 contiguous amino acids from a monomeric subunit protein capable of self-assembling to form a nanoparticle, joined to at least one immunogenic portion from a first Epstein-Barr Virus (EBV) envelope protein selected from the group consisting of gp350, gH, gL and gp42.

14. The recombinant protein of claim 13, wherein the monomeric subunit protein is selected from the group consisting of a monomeric ferritin subunit protein, a monomeric encapsulin protein, a monomeric 03-33 protein, a monomeric SOR. protein, a monomeric LS protein and a monomeric PDC protein.

15. The recombinant protein of claim 13, wherein the monomeric subunit protein is selected from the group consisting of:
a) a monomeric subunit protein comprising at least 25 contiguous amino acids of an amino acid sequence selected from the group consisting of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26 and SEQ ID NO:29; and,
b) a monomeric subunit protein comprising an amino acid sequence at least about 80% identical to an amino acid sequence selected from the group consisting of SEQ ID NO:2, SEQ ID NO:5, SEQ ID NO:8, SEQ ID NO:11, SEQ ID NO. 14, SEQ ID NO:17, SEQ ID NO:20, SEQ ID NO:23, SEQ ID NO:26 and SEQ ID NO:29.

16. The recombinant protein of claim 13, wherein the monomeric subunit protein comprises an amino acid sequence selected from the group consisting of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26 and SEQ ID NO:29.

17. The recombinant protein of claim 13, wherein the at least one immunogenic portion is selected from the group consisting of:
a) an immunogenic portion comprising at least 25 contiguous amino acids from an amino acid sequence selected from the group consisting of SEQ ID NO:32, SEQ ID NO:35, SEQ ID NO:38, SEQ ID NO:41, SEQ ID NO:44, SEQ ID NO:47, SEQ ID NQ:50, SEQ ID NO:53, SEQ ID NO:6, SEQ ID NO:59, SEQ ID NO:62, SEQ ID NO:65 and SEQ ID NO:68;
b) a immunogenic portion comprising an amino acid sequence at least 80% identical to an amino acid sequence selected from the group consisting of SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO 62, SEQ ID NO:65 and SEQ ID NO:68
c) an immunogenic portion comprising at least one domain selected from the group consisting of EBV gp350 Domain I, EBV gp350 Domain II and EBV gp350 Domain III; and,
d) an immunogenic portion comprising the EBV gp350 CR2-binding site.

18. The recombinant protein of claim 13, wherein the at least one immunogenic portion comprises an amino acid sequence selected from the group consisting of SEQ ID NO:32, SEQ ID NO:35, SEQ ID NO:38, SEQ ID NO:41, SEQ ID NO:44, SEQ ID NO:47, SEQ ID NQ:50, SEQ ID NO:53, SEQ ID NO:56, SEQ ID NO:59, SEQ ID NO:62, SEQ ID NO:65 and SEQ ID NO:68.

19. The recombinant protein of claim 13, wherein the recombinant protein comprises an amino acid sequence at least 80% identical to a sequence selected from the group consisting of SEQ ID NO:71, SEQ ID NO:74, SEQ ID NO:77, SEQ ID NO.80, SEQ ID NO 83, SEQ ID NO 86, SEQ ID NO 89, SEQ ID NO 92, SEQ ID NO:95, SEQ ID NO:98, SEQ ID NO: 101, SEQ ID NO:104, SEQ ID NO:128, SEQ ID NO:130, SEQ ID NO:134 and SEQ ID NO: 146.

20. A nanoparticle comprising the recombinant protein of claim 13.

21. A kit comprising the recombinant protein of claim 13.


SEQUENCE LISTING (146 sequences)

SEQ ID NO: 1 - DNA, 504 bp, Helicobacter pylori
  source 1..504: mol_type = other DNA
atgttatcaaaagacatcattaagttgctaaacgaacaagtgaataaggaaatgaactcttccaacttgtatatgagcatgagttcatggtgctatacccatagcttagatggcgcggggcttttcttgtttgaccatgcggctgaagaatacgagcatgctaaaaagcttattatcttcttgaatgaaaacaatgtgcctgtgcaattgaccagcatcagcgcgcctgagcataagtttgaaggtttgactcaaattttccaaaaagcctatgaacatgagcaacacatcagcgagtctattaacaatatcgtagatcacgccataaaaagcaaagatcatgcgactttcaatttcttgcaatggtatgtggctgaacagcatgaagaagaagtgcttttcaaggatattttggataaaattgagttgattggtaatgaaaaccatggcttgtatttagccgatcagtatgtcaaagggatcgctaaaagcaggaaatcttaa

SEQ ID NO: 2 - protein, 167 aa, Helicobacter pylori
MLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKVELIGNENHGLYLADQYVKGIAKSRKS

SEQ ID NO: 3 - DNA, 504 bp, Helicobacter pylori
  source 1..504: mol_type = other DNA
ttaagatttcctgcttttagcgatccctttgacatactgatcggctaaatacaagccatggttttcattaccaatcaactcaattttatccaaaatatccttgaaaagcacttcttcttcatgctgttcagccacataccattgcaagaaattgaaagtcgcatgatctttgctttttatggcgtgatctacgatattgttaatagactcgctgatgtgttgctcatgttcataggctttttggaaaatttgagtcaaaccttcaaacttatgctcaggcgcgctgatgctggtcaattgcacaggcacattgttttcattcaagaagataataagctttttagcatgctcgtattcttcagccgcatggtcaaacaagaaaagccccgcgccatctaagctatgggtatagcaccatgaactcatgctcatatacaagttggaagagttcatttccttattcacttgttcgtttagcaacttaatgatgtcttttgataacat

SEQ ID NO: 4 - skipped

SEQ ID NO: 5 - protein, 165 aa, Escherichia coli
MLKPEMIEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDAQN

SEQ ID NO: 6-7 - skipped (2 entries)

SEQ ID NO: 8 - protein, 173 aa, Rana catesbeiana
MESQVRQNFHQDCEAGLNRTVNLKFHSSYVYLSMASYFNRDDVALSNFAKFFRERSEEEKEHAEKLIEYQNQRGGRVFLQSVEKPERDDWANGLEALQTALKLQKSVNQALLDLHAVAADKSDPHMTDFLESPYLSESVETIKKLGDHITSLKKLWSSHPGMAEYLFNKHTLG

SEQ ID NO: 9 - skipped

SEQ ID NO: 10 - DNA, 531 bp, synthetic construct
  misc_feature 1..531: note = Synthetic
  source 1..531: mol_type = other DNA
tccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcc

SEQ ID NO: 11 - protein, 177 aa, synthetic construct
  REGION 1..177: note = Synthetic
SGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 12 - DNA, 531 bp, synthetic construct
  misc_feature 1..531: note = Synthetic
  source 1..531: mol_type = other DNA
ggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccgga

SEQ ID NO: 13 - DNA, 516 bp, synthetic construct
  misc_feature 1..516: note = Synthetic
  source 1..516: mol_type = other DNA
tccggagagagccaggtgaggcagaacttcaagcccgagatggaggagaagctgaacgagcagatgaacctggagctgtacagcagcctgctgtaccagcagatgagcgcctggtgcagctaccacaccttcgagggcgccgccgccttcctgaggaggcacgcccaggaggagatgacccacatgcagaggctgttcgactacctgaccgacaccggcaacctgcccaggatcaacaccgtggagagccccttcgccgagtacagcagcctggacgagctgttccaggagacctacaagcacgagcagctgatcacccagaagatcaacgagctggcccacgccgccatgaccaaccaggactaccccaccttcaacttcctgcagtggtacgtgagcgagcagcacgaggaggagaagctgttcaagagcatcatcgacaagctgagcctggccggcaagagcggcgagggcctgtacttcatcgacaaggagctgagcaccctggacggatcc

SEQ ID NO: 14 - protein, 172 aa, synthetic construct
  REGION 1..172: note = Synthetic
SGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 15 - DNA, 516 bp, synthetic construct
  misc_feature 1..516: note = Synthetic
  source 1..516: mol_type = other DNA
ggatccgtccagggtgctcagctccttgtcgatgaagtacaggccctcgccgctcttgccggccaggctcagcttgtcgatgatgctcttgaacagcttctcctcctcgtgctgctcgctcacgtaccactgcaggaagttgaaggtggggtagtcctggttggtcatggcggcgtgggccagctcgttgatcttctgggtgatcagctgctcgtgcttgtaggtctcctggaacagctcgtccaggctgctgtactcggcgaaggggctctccacggtgttgatcctgggcaggttgccggtgtcggtcaggtagtcgaacagcctctgcatgtgggtcatctcctcctgggcgtgcctcctcaggaaggcggcggcgccctcgaaggtgtggtagctgcaccaggcgctcatctgctggtacagcaggctgctgtacagctccaggttcatctgctcgttcagcttctcctccatctcgggcttgaagttctgcctcacctggctctctccgga

SEQ ID NO: 16 - DNA, 792 bp, Thermotoga maritima
  source 1..792: mol_type = other DNA
atggagttcctgaagaggagcttcgcccctctgaccgagaagcagtggcaggagatcgacaacagggccagggagatcttcaagacccagctgtacggcaggaagttcgtggacgtggagggcccctacggctgggagtacgccgcccaccccctgggcgaggtggaggtgctgagcgacgagaacgaggtggtgaagtggggcctgaggaagagcctgcccctgatcgagctgagggccaccttcaccctggacctgtgggagctggacaacctggagaggggcaagcccaacgtggacctgagcagcctggaggagaccgtgaggaaggtggccgagttcgaggacgaggtgatcttcaggggctgcgagaagagcggcgtgaagggcctgctgagcttcgaggagaggaagatcgagtgcggcagcacccccaaggacctgctggaggccatcgtgagggccctgagcatcttcagcaaggacggcatcgagggcccctacaccctggtgatcaacaccgacaggtggatcaacttcctgaaggaggaggccggccactaccccctggagaagagggtggaggagtgcctgaggggcggcaagatcatcaccacccccaggatcgaggacgccctggtggtgagcgagaggggcggcgacttcaagctgatcctgggccaggacctgagcatcggctacgaggacagggagaaggacgccgtgaggctgttcatcaccgagaccttcaccttccaggtggtgaaccccgaggccctgatcctgctgaag

SEQ ID NO: 17 - protein, 264 aa, Thermotoga maritima
MEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLK

SEQ ID NO: 18 - DNA, 792 bp, Thermotoga maritima
  source 1..792: mol_type = other DNA
cttcagcaggatcagggcctcggggttcaccacctggaaggtgaaggtctcggtgatgaacagcctcacggcgtccttctccctgtcctcgtagccgatgctcaggtcctggcccaggatcagcttgaagtcgccgcccctctcgctcaccaccagggcgtcctcgatcctgggggtggtgatgatcttgccgcccctcaggcactcctccaccctcttctccagggggtagtggccggcctcctccttcaggaagttgatccacctgtcggtgttgatcaccagggtgtaggggccctcgatgccgtccttgctgaagatgctcagggccctcacgatggcctccagcaggtccttgggggtgctgccgcactcgatcttcctctcctcgaagctcagcaggcccttcacgccgctcttctcgcagcccctgaagatcacctcgtcctcgaactcggccaccttcctcacggtctcctccaggctgctcaggtccacgttgggcttgcccctctccaggttgtccagctcccacaggtccagggtgaaggtggccctcagctcgatcaggggcaggctcttcctcaggccccacttcaccacctcgttctcgtcgctcagcacctccacctcgcccagggggtgggcggcgtactcccagccgtaggggccctccacgtccacgaacttcctgccgtacagctgggtcttgaagatctccctggccctgttgtcgatctcctgccactgcttctcggtcagaggggcgaagctcctcttcaggaactccat

SEQ ID NO: 19 - skipped

SEQ ID NO: 20 - protein, 192 aa, synthetic construct
  REGION 1..192: note = Synthetic
MSQAIGILELTSIAAGMELGDAMLKSANVDLLVSKTISPGKFLLMLGGDIGAIQQAIETGTSQAGELLVDSLVLANIHPSVLPAISGLNSVDKRQAVGIVETWSVAACISAADRAVKGSNVTLVRVHMAFGIGGKCYMVVAGDVSDVALAVTVASSSAGAYGLLVYASLIPRPHEAMWRQMVEGLEHHHHHH

SEQ ID NO: 21-22 - skipped (2 entries)

SEQ ID NO: 23 - protein, 318 aa, Acidianus ambivalens
  VARIANT 31: note = X is any naturally occurring amino acid
MPKPYVAINMAELKNEPKTFEMFASVGPKVXMVTARHPGFVGFQNHIQIGILPFGNRYGGAKMDMTKESSTVRVLQYTFWKDWKDHEEMHRQNWSYLFRLCYSCASQMIWGPWEPIYEIIYANMPINTEMTDFTAVVGKKFAEGKPLDIPVISQPYGKRVVAFAEHSVIPGKEKQFEDAIVRTLEMLKKAPGFLGAMVLKEIGVSGIGSMQFGAKGFHQVLENPGSLEPDPNNVMYSVPEAKNTPQQYIVHVEWANTDALMFGMGRVLLYPELRQVHDEVLDTLVYGPYIRILNPMMEGTFWREYLNEQAWRHPQFGG

SEQ ID NO: 24-25 - skipped (2 entries)

SEQ ID NO: 26 - protein, 154 aa, Aquifex pyrophilus
MQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 27-28 - skipped (2 entries)

SEQ ID NO: 29 - protein, 242 aa, Bacillus stearothermophilus
AAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRDGEIVAAPMLALSLSFDHRMIDGATAQKALNHIKRLLSDPELLLM

SEQ ID NO: 30 - skipped

SEQ ID NO: 31 - DNA, 2721 bp, Epstein Barr Virus
  source 1..2721: mol_type = other DNA
atggaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgagtccactaccacatctcctaccctgaacactaccgggtttgccgaccccaatacaactaccggactgcctagctccacccatgtgccaacaaacctgactgcaccagcatccaccggacctacagtgtctactgccgatgtcaccagtcccacacctgccggaacaacttctggcgctagtcccgtgaccccatcacccagcccttgggacaatgggacagagagtaaggcccctgatatgacttctagtacctcaccagtcaccacaccaacccccaacgcaacaagccctactccagccgtgactacccccacacctaatgctaccagcccaacacccgcagtgacaactcctaccccaaacgccacttccccaaccctggggaagacatcacccactagcgccgtgaccacacccacccctaatgctacctctcctacactgggaaaaacttccccaacctctgcagtgactaccccaacccccaacgccacaagccccactctgggcaagaccagtcctacatcagctgtcacaactcctaccccaaatgcaactgggccaaccgtgggagagacatccccccaggctaacgcaacaaatcacactctgggaggcaccagtcccacacctgtggtcacctcacagcccaagaacgccacaagcgctgtgaccacaggccagcataatatcacatcaagctccacttctagtatgagcctgcgcccttcaagcaacccagagacactgtccccatctactagtgacaattcaaccagccacatgcctctgctgacatctgcacatccaactgggggagaaaacatcactcaggtcacccccgcctccatttctacccaccatgtgtccacatcctctccagcaccccgacctggaactaccagccaggcatccggaccaggaaatagttcaaccagcacaaagcctggcgaggtgaacgtcacaaaagggactccccctcagaatgctacctcacctcaggcaccaagcggccagaaaacagctgtgcctactgtcacctccacaggcgggaaggcaaactctacaactggaggcaaacacaccacagggcatggagctcgcactagcaccgaaccaactaccgactacgggggagattccacaactccaaggcccagatacaatgccaccacatatctgccaccctctaccagctccaagctgcgacccagatggacattcactagtcctccagtgactaccgcacaggctacagtgccagtcccacctacttctcagcctagattttctaacctgagtatgctggtgctgcagtgggcaagcctggcagtcctgaccctgctgctgctgctggtcatggctgactgtgcattccggagaaacctgtccacttcacacacttacaccacccccccttacgatgacgcagagacttatgtc

SEQ ID NO: 32 - protein, 907 aa, Epstein Barr Virus
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 33 - DNA, 2721 bp, Epstein Barr Virus
  source 1..2721: mol_type = other DNA
gacataagtctctgcgtcatcgtaagggggggtggtgtaagtgtgtgaagtggacaggtttctccggaatgcacagtcagccatgaccagcagcagcagcagggtcaggactgccaggcttgcccactgcagcaccagcatactcaggttagaaaatctaggctgagaagtaggtgggactggcactgtagcctgtgcggtagtcactggaggactagtgaatgtccatctgggtcgcagcttggagctggtagagggtggcagatatgtggtggcattgtatctgggccttggagttgtggaatctcccccgtagtcggtagttggttcggtgctagtgcgagctccatgccctgtggtgtgtttgcctccagttgtagagtttgccttcccgcctgtggaggtgacagtaggcacagctgttttctggccgcttggtgcctgaggtgaggtagcattctgagggggagtcccttttgtgacgttcacctcgccaggctttgtgctggttgaactatttcctggtccggatgcctggctggtagttccaggtcggggtgctggagaggatgtggacacatggtgggtagaaatggaggcgggggtgacctgagtgatgttttctcccccagttggatgtgcagatgtcagcagaggcatgtggctggttgaattgtcactagtagatggggacagtgtctctgggttgcttgaagggcgcaggctcatactagaagtggagcttgatgtgatattatgctggcctgtggtcacagcgcttgtggcgttcttgggctgtgaggtgaccacaggtgtgggactggtgcctcccagagtgtgatttgttgcgttagcctggggggatgtctctcccacggttggcccagttgcatttggggtaggagttgtgacagctgatgtaggactggtcttgcccagagtggggcttgtggcgttgggggttggggtagtcactgcagaggttggggaagtttttcccagtgtaggagaggtagcattaggggtgggtgtggtcacggcgctagtgggtgatgtcttccccagggttggggaagtggcgtttggggtaggagttgtcactgcgggtgttgggctggtagcattaggtgtgggggtagtcacggctggagtagggcttgttgcgttgggggttggtgtggtgactggtgaggtactagaagtcatatcaggggccttactctctgtcccattgtcccaagggctgggtgatggggtcacgggactagcgccagaagttgttccggcaggtgtgggactggtgacatcggcagtagacactgtaggtccggtggatgctggtgcagtcaggtttgttggcacatgggtggagctaggcagtccggtagttgtattggggtcggcaaacccggtagtgttcagggtaggagatgtggtagtggactcgggagctttgctgaagatcactttgtgtgtggtagttgttgcgttagtagctgtccgggtaatgatcagggtttttggggcagtccccaggccggacactgtaatatcaaaggttcgattactagcgaaggcgccagagatgttttcacacccgctaggggtgcctgaagtcagtgtccatttgcacttaaaatcggtctcagtattgttgggccaggcccagaatgctgtgacggtcacatttggggagttagcgtcttcgcttgtgaccataggcacggaataagtggcattatcgcccacgtaggtaatgtcggtagtgttagtgggcatatcctggctggctgggatctcgtctgagaacacaatattactctggatgcaatagtcccctccagaagcctttggtccgtttccggaataaaacacgtacagaatgctattgttgcccaggaagcgtgagactggcctaggggtcagtctcaggctataagcgtagccggttccgggaattggagtggccacgggactagtagatgtcaggattcccccacttgggacgtgagattcgtagccggaacaggtgatattaaacttgttatcgccaggcagcacctggctaatttctccatcctccatgatgcattcaatgtcgatctcatttcccagcatctcggttttcacggagaagttgctatcctgggcgcttgtgggcagtgacagtggcagggtcacgtccagcccctgtgcccggaccactgcggtaatgttagtgctattacagttgtcccacttaatataagggacggtttcggggatcaggtacacaggattctgcatctcggcatggtggcaccacatggtgccaaacacatcctggaagtagacgtccactgattccagactgacctgctgttcctctcctgtggtcacattgatgggcagtttcttggacctcatggtcagagccagttctcctgctcccagcagctccagcagaaacagatttgttgcgttctcgctgcctccgaaagcccctctgggctgatacacggcttttgtatgtggggtcagctggccaaaatccaggtccagctggtgtttcttcccgcccacatcgaaattaatggtgacgttcacgtcggctgtacagacgttgcaggtggggtaaaatgggaactcagggatttccacattgaaaaatccagggtcctccccggtcagatgaatcaggctctgaatagtgtactggcacaccagcagggcggcttccat

SEQ ID NO: 34 - DNA, 2577 bp, Epstein Barr Virus
  source 1..2577: mol_type = other DNA
gaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgagtccactaccacatctcctaccctgaacactaccgggtttgccgaccccaatacaactaccggactgcctagctccacccatgtgccaacaaacctgactgcaccagcatccaccggacctacagtgtctactgccgatgtcaccagtcccacacctgccggaacaacttctggcgctagtcccgtgaccccatcacccagcccttgggacaatgggacagagagtaaggcccctgatatgacttctagtacctcaccagtcaccacaccaacccccaacgcaacaagccctactccagccgtgactacccccacacctaatgctaccagcccaacacccgcagtgacaactcctaccccaaacgccacttccccaaccctggggaagacatcacccactagcgccgtgaccacacccacccctaatgctacctctcctacactgggaaaaacttccccaacctctgcagtgactaccccaacccccaacgccacaagccccactctgggcaagaccagtcctacatcagctgtcacaactcctaccccaaatgcaactgggccaaccgtgggagagacatccccccaggctaacgcaacaaatcacactctgggaggcaccagtcccacacctgtggtcacctcacagcccaagaacgccacaagcgctgtgaccacaggccagcataatatcacatcaagctccacttctagtatgagcctgcgcccttcaagcaacccagagacactgtccccatctactagtgacaattcaaccagccacatgcctctgctgacatctgcacatccaactgggggagaaaacatcactcaggtcacccccgcctccatttctacccaccatgtgtccacatcctctccagcaccccgacctggaactaccagccaggcatccggaccaggaaatagttcaaccagcacaaagcctggcgaggtgaacgtcacaaaagggactccccctcagaatgctacctcacctcaggcaccaagcggccagaaaacagctgtgcctactgtcacctccacaggcgggaaggcaaactctacaactggaggcaaacacaccacagggcatggagctcgcactagcaccgaaccaactaccgactacgggggagattccacaactccaaggcccagatacaatgccaccacatatctgccaccctctaccagctccaagctgcgacccagatggacattcactagtcctccagtgactaccgcacaggctacagtgccagtcccacctacttctcagcctagattttctaacctgagt

SEQ ID NO: 35 - protein, 859 aa, Epstein Barr Virus
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLS

SEQ ID NO: 36 - DNA, 2577 bp, Epstein Barr Virus
  source 1..2577: mol_type = other DNA
actcaggttagaaaatctaggctgagaagtaggtgggactggcactgtagcctgtgcggtagtcactggaggactagtgaatgtccatctgggtcgcagcttggagctggtagagggtggcagatatgtggtggcattgtatctgggccttggagttgtggaatctcccccgtagtcggtagttggttcggtgctagtgcgagctccatgccctgtggtgtgtttgcctccagttgtagagtttgccttcccgcctgtggaggtgacagtaggcacagctgttttctggccgcttggtgcctgaggtgaggtagcattctgagggggagtcccttttgtgacgttcacctcgccaggctttgtgctggttgaactatttcctggtccggatgcctggctggtagttccaggtcggggtgctggagaggatgtggacacatggtgggtagaaatggaggcgggggtgacctgagtgatgttttctcccccagttggatgtgcagatgtcagcagaggcatgtggctggttgaattgtcactagtagatggggacagtgtctctgggttgcttgaagggcgcaggctcatactagaagtggagcttgatgtgatattatgctggcctgtggtcacagcgcttgtggcgttcttgggctgtgaggtgaccacaggtgtgggactggtgcctcccagagtgtgatttgttgcgttagcctggggggatgtctctcccacggttggcccagttgcatttggggtaggagttgtgacagctgatgtaggactggtcttgcccagagtggggcttgtggcgttgggggttggggtagtcactgcagaggttggggaagtttttcccagtgtaggagaggtagcattaggggtgggtgtggtcacggcgctagtgggtgatgtcttccccagggttggggaagtggcgtttggggtaggagttgtcactgcgggtgttgggctggtagcattaggtgtgggggtagtcacggctggagtagggcttgttgcgttgggggttggtgtggtgactggtgaggtactagaagtcatatcaggggccttactctctgtcccattgtcccaagggctgggtgatggggtcacgggactagcgccagaagttgttccggcaggtgtgggactggtgacatcggcagtagacactgtaggtccggtggatgctggtgcagtcaggtttgttggcacatgggtggagctaggcagtccggtagttgtattggggtcggcaaacccggtagtgttcagggtaggagatgtggtagtggactcgggagctttgctgaagatcactttgtgtgtggtagttgttgcgttagtagctgtccgggtaatgatcagggtttttggggcagtccccaggccggacactgtaatatcaaaggttcgattactagcgaaggcgccagagatgttttcacacccgctaggggtgcctgaagtcagtgtccatttgcacttaaaatcggtctcagtattgttgggccaggcccagaatgctgtgacggtcacatttggggagttagcgtcttcgcttgtgaccataggcacggaataagtggcattatcgcccacgtaggtaatgtcggtagtgttagtgggcatatcctggctggctgggatctcgtctgagaacacaatattactctggatgcaatagtcccctccagaagcctttggtccgtttccggaataaaacacgtacagaatgctattgttgcccaggaagcgtgagactggcctaggggtcagtctcaggctataagcgtagccggttccgggaattggagtggccacgggactagtagatgtcaggattcccccacttgggacgtgagattcgtagccggaacaggtgatattaaacttgttatcgccaggcagcacctggctaatttctccatcctccatgatgcattcaatgtcgatctcatttcccagcatctcggttttcacggagaagttgctatcctgggcgcttgtgggcagtgacagtggcagggtcacgtccagcccctgtgcccggaccactgcggtaatgttagtgctattacagttgtcccacttaatataagggacggtttcggggatcaggtacacaggattctgcatctcggcatggtggcaccacatggtgccaaacacatcctggaagtagacgtccactgattccagactgacctgctgttcctctcctgtggtcacattgatgggcagtttcttggacctcatggtcagagccagttctcctgctcccagcagctccagcagaaacagatttgttgcgttctcgctgcctccgaaagcccctctgggctgatacacggcttttgtatgtggggtcagctggccaaaatccaggtccagctggtgtttcttcccgcccacatcgaaattaatggtgacgttcacgtcggctgtacagacgttgcaggtggggtaaaatgggaactcagggatttccacattgaaaaatccagggtcctccccggtcagatgaatcaggctctgaatagtgtactggcacaccagcagggcggcttc

SEQ ID NO: 37 - DNA, 1407 bp, Epstein Barr Virus
  source 1..1407: mol_type = other DNA
gaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgagtccactaccacatctcctaccctgaacactaccgggtttgccgaccccaatacaactaccggactgcctagctccacccatgtgccaacaaacctgactgcaccagcatccaccggacctacagtgtctact

SEQ ID NO: 38 - protein, 469 aa, Epstein Barr Virus
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVST

SEQ ID NO: 39 - DNA, 1407 bp, Epstein Barr Virus
  source 1..1407: mol_type = other DNA
agtagacactgtaggtccggtggatgctggtgcagtcaggtttgttggcacatgggtggagctaggcagtccggtagttgtattggggtcggcaaacccggtagtgttcagggtaggagatgtggtagtggactcgggagctttgctgaagatcactttgtgtgtggtagttgttgcgttagtagctgtccgggtaatgatcagggtttttggggcagtccccaggccggacactgtaatatcaaaggttcgattactagcgaaggcgccagagatgttttcacacccgctaggggtgcctgaagtcagtgtccatttgcacttaaaatcggtctcagtattgttgggccaggcccagaatgctgtgacggtcacatttggggagttagcgtcttcgcttgtgaccataggcacggaataagtggcattatcgcccacgtaggtaatgtcggtagtgttagtgggcatatcctggctggctgggatctcgtctgagaacacaatattactctggatgcaatagtcccctccagaagcctttggtccgtttccggaataaaacacgtacagaatgctattgttgcccaggaagcgtgagactggcctaggggtcagtctcaggctataagcgtagccggttccgggaattggagtggccacgggactagtagatgtcaggattcccccacttgggacgtgagattcgtagccggaacaggtgatattaaacttgttatcgccaggcagcacctggctaatttctccatcctccatgatgcattcaatgtcgatctcatttcccagcatctcggttttcacggagaagttgctatcctgggcgcttgtgggcagtgacagtggcagggtcacgtccagcccctgtgcccggaccactgcggtaatgttagtgctattacagttgtcccacttaatataagggacggtttcggggatcaggtacacaggattctgcatctcggcatggtggcaccacatggtgccaaacacatcctggaagtagacgtccactgattccagactgacctgctgttcctctcctgtggtcacattgatgggcagtttcttggacctcatggtcagagccagttctcctgctcccagcagctccagcagaaacagatttgttgcgttctcgctgcctccgaaagcccctctgggctgatacacggcttttgtatgtggggtcagctggccaaaatccaggtccagctggtgtttcttcccgcccacatcgaaattaatggtgacgttcacgtcggctgtacagacgttgcaggtggggtaaaatgggaactcagggatttccacattgaaaaatccagggtcctccccggtcagatgaatcaggctctgaatagtgtactggcacaccagcagggcggcttc

SEQ ID NO: 40 - DNA, 450 bp, Epstein Barr Virus
  source 1..450: mol_type = other DNA
gccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatc

SEQ ID NO: 41 - protein, 150 aa, Epstein Barr Virus
ALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLI

SEQ ID NO: 42 - DNA, 450 bp, Epstein Barr Virus
  source 1..450: mol_type = other DNA
gatcaggtacacaggattctgcatctcggcatggtggcaccacatggtgccaaacacatcctggaagtagacgtccactgattccagactgacctgctgttcctctcctgtggtcacattgatgggcagtttcttggacctcatggtcagagccagttctcctgctcccagcagctccagcagaaacagatttgttgcgttctcgctgcctccgaaagcccctctgggctgatacacggcttttgtatgtggggtcagctggccaaaatccaggtccagctggtgtttcttcccgcccacatcgaaattaatggtgacgttcacgtcggctgtacagacgttgcaggtggggtaaaatgggaactcagggatttccacattgaaaaatccagggtcctccccggtcagatgaatcaggctctgaatagtgtactggcacaccagcagggc

SEQ ID NO: 43 - DNA, 423 bp, Epstein Barr Virus
  source 1..423: mol_type = other DNA
tgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttc

SEQ ID NO: 44 - protein, 141 aa, Epstein Barr Virus
CNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVF

SEQ ID NO: 45 - DNA, 423 bp, Epstein Barr Virus
  source 1..423: mol_type = other DNA
gaacacaatattactctggatgcaatagtcccctccagaagcctttggtccgtttccggaataaaacacgtacagaatgctattgttgcccaggaagcgtgagactggcctaggggtcagtctcaggctataagcgtagccggttccgggaattggagtggccacgggactagtagatgtcaggattcccccacttgggacgtgagattcgtagccggaacaggtgatattaaacttgttatcgccaggcagcacctggctaatttctccatcctccatgatgcattcaatgtcgatctcatttcccagcatctcggttttcacggagaagttgctatcctgggcgcttgtgggcagtgacagtggcagggtcacgtccagcccctgtgcccggaccactgcggtaatgttagtgctattaca

SEQ ID NO: 46 - DNA, 330 bp, Epstein Barr Virus
  source 1..330: mol_type = other DNA
actaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgag

SEQ ID NO: 47 - protein, 110 aa, Epstein Barr Virus
TNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPE

SEQ ID NO: 48 - DNA, 330 bp, Epstein Barr Virus
  source 1..330: mol_type = other DNA
ctcgggagctttgctgaagatcactttgtgtgtggtagttgttgcgttagtagctgtccgggtaatgatcagggtttttggggcagtccccaggccggacactgtaatatcaaaggttcgattactagcgaaggcgccagagatgttttcacacccgctaggggtgcctgaagtcagtgtccatttgcacttaaaatcggtctcagtattgttgggccaggcccagaatgctgtgacggtcacatttggggagttagcgtcttcgcttgtgaccataggcacggaataagtggcattatcgcccacgtaggtaatgtcggtagtgttagt

SEQ ID NO: 49 - DNA, 948 bp, Epstein Barr Virus
  source 1..948: mol_type = other DNA
gaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccact

SEQ ID NO: 50 - protein, 316 aa, Epstein Barr Virus
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPT

SEQ ID NO: 51 - DNA, 948 bp, Epstein Barr Virus
  source 1..948: mol_type = other DNA
agtgggcatatcctggctggctgggatctcgtctgagaacacaatattactctggatgcaatagtcccctccagaagcctttggtccgtttccggaataaaacacgtacagaatgctattgttgcccaggaagcgtgagactggcctaggggtcagtctcaggctataagcgtagccggttccgggaattggagtggccacgggactagtagatgtcaggattcccccacttgggacgtgagattcgtagccggaacaggtgatattaaacttgttatcgccaggcagcacctggctaatttctccatcctccatgatgcattcaatgtcgatctcatttcccagcatctcggttttcacggagaagttgctatcctgggcgcttgtgggcagtgacagtggcagggtcacgtccagcccctgtgcccggaccactgcggtaatgttagtgctattacagttgtcccacttaatataagggacggtttcggggatcaggtacacaggattctgcatctcggcatggtggcaccacatggtgccaaacacatcctggaagtagacgtccactgattccagactgacctgctgttcctctcctgtggtcacattgatgggcagtttcttggacctcatggtcagagccagttctcctgctcccagcagctccagcagaaacagatttgttgcgttctcgctgcctccgaaagcccctctgggctgatacacggcttttgtatgtggggtcagctggccaaaatccaggtccagctggtgtttcttcccgcccacatcgaaattaatggtgacgttcacgtcggctgtacagacgttgcaggtggggtaaaatgggaactcagggatttccacattgaaaaatccagggtcctccccggtcagatgaatcaggctctgaatagtgtactggcacaccagcagggcggcttc

SEQ ID NO: 52 - DNA, 1272 bp, Epstein Barr Virus
  source 1..1272: mol_type = other DNA
gaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctccc

SEQ ID NO: 53 - protein, 424 aa, Epstein Barr Virus
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAP

SEQ ID NO: 54 - DNA, 1272 bp, Epstein Barr Virus
  source 1..1272: mol_type = other DNA
gggagctttgctgaagatcactttgtgtgtggtagttgttgcgttagtagctgtccgggtaatgatcagggtttttggggcagtccccaggccggacactgtaatatcaaaggttcgattactagcgaaggcgccagagatgttttcacacccgctaggggtgcctgaagtcagtgtccatttgcacttaaaatcggtctcagtattgttgggccaggcccagaatgctgtgacggtcacatttggggagttagcgtcttcgcttgtgaccataggcacggaataagtggcattatcgcccacgtaggtaatgtcggtagtgttagtgggcatatcctggctggctgggatctcgtctgagaacacaatattactctggatgcaatagtcccctccagaagcctttggtccgtttccggaataaaacacgtacagaatgctattgttgcccaggaagcgtgagactggcctaggggtcagtctcaggctataagcgtagccggttccgggaattggagtggccacgggactagtagatgtcaggattcccccacttgggacgtgagattcgtagccggaacaggtgatattaaacttgttatcgccaggcagcacctggctaatttctccatcctccatgatgcattcaatgtcgatctcatttcccagcatctcggttttcacggagaagttgctatcctgggcgcttgtgggcagtgacagtggcagggtcacgtccagcccctgtgcccggaccactgcggtaatgttagtgctattacagttgtcccacttaatataagggacggtttcggggatcaggtacacaggattctgcatctcggcatggtggcaccacatggtgccaaacacatcctggaagtagacgtccactgattccagactgacctgctgttcctctcctgtggtcacattgatgggcagtttcttggacctcatggtcagagccagttctcctgctcccagcagctccagcagaaacagatttgttgcgttctcgctgcctccgaaagcccctctgggctgatacacggcttttgtatgtggggtcagctggccaaaatccaggtccagctggtgtttcttcccgcccacatcgaaattaatggtgacgttcacgtcggctgtacagacgttgcaggtggggtaaaatgggaactcagggatttccacattgaaaaatccagggtcctccccggtcagatgaatcaggctctgaatagtgtactggcacaccagcagggcggcttc

SEQ ID NO: 55-60 - skipped (6 entries)

SEQ ID NO: 61 - DNA, 2118 bp, Epstein Barr Virus
  source 1..2118: mol_type = other DNA
atgcagctcctgtgcgtgttctgtctggtgctgctgtgggaagtgggagccgcttctctgagtgaggtgaagctgcacctggacattgaaggccacgcctcccattacactatcccttggaccgagctgatggctaaagtgccaggactgtctcctgaggctctgtggcgggaagctaatgtgaccgaggatctggcctctatgctgaacagatacaagctgatctataaaaccagtggcacactggggattgctctggctgagccagtggacatccccgccgtgtcagaaggaagcatgcaggtggatgctagtaaggtgcatccaggggtgattagcggactgaacagcccagcttgcatgctgagcgctcctctggagaaacagctcttctactatatcggcaccatgctgcctaatacacggccacacagctacgtgttttatcagctcagatgtcatctgtcctacgtggccctgtctattaacggggacaagttccagtatacaggagctatgacttccaaatttctgatgggaacttacaagcgggtgaccgagaaaggcgatgaacacgtgctgtctctggtgttcgggaagacaaaagacctgcccgatctgagaggacccttttcctacccttctctgactagtgcccagtcaggcgactatagcctggtgatcgtgaccacattcgtgcactacgctaacttccataattattttgtgcccaatctgaaggatatgttttcccgggccgtgaccatgacagccgcttcttacgctagatatgtgctgcagaagctggtgctgctggagatgaaaggcgggtgccgggagcctgaactggacactgaaaccctgactaccatgttcgaggtgtccgtggccttctttaaagtgggacacgctgtgggagagacaggaaacggatgcgtggacctgagatggctggccaagagcttctttgaactgaccgtgctgaaagatatcattggaatctgttacggcgccacagtgaaaggaatgcagagctatggcctggagaggctggccgctatgctgatggccaccgtgaagatggaggaactgggccacctgacaactgagaaacaggaatacgctctgaggctggctaccgtgggatacccaaaggccggggtgtattccggactgattggaggcgccacatctgtgctgctgagtgcttataataggcacccactgttccagcccctgcatacagtgatgcgcgagactctgtttatcgggtctcatgtggtgctgcgggaactgagactgaatgtgaccacacagggacccaacctggccctgtaccagctcctgagtactgccctgtgctcagctctggagattggagaagtgctgaggggactggccctggggaccgagtcaggactgttcagcccttgttatctgtcactgaggtttgacctgactcgcgataagctgctgagcatggccccacaggaagctaccctggaccaggccgctgtgagcaatgccgtggatggattcctgggcaggctgtccctggagagggaagaccgcgatgcctggcacctgccagcttacaagtgcgtggaccgcctggataaagtgctgatgatcattcccctgatcaacgtgaccttcatcattagctccgacagggaagtgagaggcagcgctctgtacgaagcttccactacctatctgtctagttcactgtttctgtcacctgtgattatgaataagtgtagccagggagctgtggctggagagcccagacagatcccaaagattcagaacttcacacgcactcagaaaagttgcatcttctgtggctttgccctgctgtcatacgatgagaaagaagggctggagacaactacctatattacatctcaggaagtgcagaacagtatcctgagctccaattacttcgactttgataacctgcacgtgcattatctgctgctgacaactaacggcaccgtgatggagatcgctggactgtacgaggaaagggctcacgtggtgctggctatcattctgtatttcatcgcctttgctctgggcatttttctggtgcataagatcgtgatgttctttctg

SEQ ID NO: 62 - protein, 706 aa, Epstein Barr Virus
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHVVLAIILYFIAFALGIFLVHKIVMFFL

SEQ ID NO: 63 - DNA, 2118 bp, Epstein Barr Virus
  source 1..2118: mol_type = other DNA
cagaaagaacatcacgatcttatgcaccagaaaaatgcccagagcaaaggcgatgaaatacagaatgatagccagcaccacgtgagccctttcctcgtacagtccagcgatctccatcacggtgccgttagttgtcagcagcagataatgcacgtgcaggttatcaaagtcgaagtaattggagctcaggatactgttctgcacttcctgagatgtaatataggtagttgtctccagcccttctttctcatcgtatgacagcagggcaaagccacagaagatgcaacttttctgagtgcgtgtgaagttctgaatctttgggatctgtctgggctctccagccacagctccctggctacacttattcataatcacaggtgacagaaacagtgaactagacagataggtagtggaagcttcgtacagagcgctgcctctcacttccctgtcggagctaatgatgaaggtcacgttgatcaggggaatgatcatcagcactttatccaggcggtccacgcacttgtaagctggcaggtgccaggcatcgcggtcttccctctccagggacagcctgcccaggaatccatccacggcattgctcacagcggcctggtccagggtagcttcctgtggggccatgctcagcagcttatcgcgagtcaggtcaaacctcagtgacagataacaagggctgaacagtcctgactcggtccccagggccagtcccctcagcacttctccaatctccagagctgagcacagggcagtactcaggagctggtacagggccaggttgggtccctgtgtggtcacattcagtctcagttcccgcagcaccacatgagacccgataaacagagtctcgcgcatcactgtatgcaggggctggaacagtgggtgcctattataagcactcagcagcacagatgtggcgcctccaatcagtccggaatacaccccggcctttgggtatcccacggtagccagcctcagagcgtattcctgtttctcagttgtcaggtggcccagttcctccatcttcacggtggccatcagcatagcggccagcctctccaggccatagctctgcattcctttcactgtggcgccgtaacagattccaatgatatctttcagcacggtcagttcaaagaagctcttggccagccatctcaggtccacgcatccgtttcctgtctctcccacagcgtgtcccactttaaagaaggccacggacacctcgaacatggtagtcagggtttcagtgtccagttcaggctcccggcacccgcctttcatctccagcagcaccagcttctgcagcacatatctagcgtaagaagcggctgtcatggtcacggcccgggaaaacatatccttcagattgggcacaaaataattatggaagttagcgtagtgcacgaatgtggtcacgatcaccaggctatagtcgcctgactgggcactagtcagagaagggtaggaaaagggtcctctcagatcgggcaggtcttttgtcttcccgaacaccagagacagcacgtgttcatcgcctttctcggtcacccgcttgtaagttcccatcagaaatttggaagtcatagctcctgtatactggaacttgtccccgttaatagacagggccacgtaggacagatgacatctgagctgataaaacacgtagctgtgtggccgtgtattaggcagcatggtgccgatatagtagaagagctgtttctccagaggagcgctcagcatgcaagctgggctgttcagtccgctaatcacccctggatgcaccttactagcatccacctgcatgcttccttctgacacggcggggatgtccactggctcagccagagcaatccccagtgtgccactggttttatagatcagcttgtatctgttcagcatagaggccagatcctcggtcacattagcttcccgccacagagcctcaggagacagtcctggcactttagccatcagctcggtccaagggatagtgtaatgggaggcgtggccttcaatgtccaggtgcagcttcacctcactcagagaagcggctcccacttcccacagcagcaccagacagaacacgcacaggagctgcat

SEQ ID NO: 64 - DNA, 411 bp, Epstein Barr Virus
  source 1..411: mol_type = other DNA
atgcgggccgtgggggtgttcctggctatctgcctggtgactatttttgtgctgccaacctggggaaactgggcttacccttgctgtcacgtgacccagctcagggcccagcatctgctggctctggagaacatcagcgacatttatctggtgtccaatcagacatgcgatgggttcagcctggcctccctgaacagccccaagaacggatctaatcagctcgtgatctcccggtgtgctaacggcctgaatgtcgtgagtttctttatctcaattctgaaaaggagctcctctgctctgacaggacacctgagggagctgctgaccacactggaaactctgtacggaagtttctcagtggaagacctgtttggcgccaacctgaatcggtatgcttggcatagaggcggg

SEQ ID NO: 65 - protein, 137 aa, Epstein Barr Virus
MRAVGVFLAICLVTIFVLPTWGNWAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGG

SEQ ID NO: 66 - DNA, 411 bp, Epstein Barr Virus
  source 1..411: mol_type = other DNA
cccgcctctatgccaagcataccgattcaggttggcgccaaacaggtcttccactgagaaacttccgtacagagtttccagtgtggtcagcagctccctcaggtgtcctgtcagagcagaggagctccttttcagaattgagataaagaaactcacgacattcaggccgttagcacaccgggagatcacgagctgattagatccgttcttggggctgttcagggaggccaggctgaacccatcgcatgtctgattggacaccagataaatgtcgctgatgttctccagagccagcagatgctgggccctgagctgggtcacgtgacagcaagggtaagcccagtttccccaggttggcagcacaaaaatagtcaccaggcagatagccaggaacacccccacggcccgcat

SEQ ID NO: 67 - DNA, 669 bp, Epstein Barr Virus
  source 1..669: mol_type = other DNA
atggtcagcttcaaacaagtgcgggtgcccctgtttactgccatcgctctggtgattgtgctgctgctggcctacttcctgccacctcgggtcagaggaggaggaagagtggccgctgccgctatcacctgggtgccaaaacctaatgtggaagtgtggcctgtggacccaccacctccagtgaactttaataagacagccgagcaggaatatggcgataaagaagtgaagctgcctcactggaccccaacactgcatacattccaggtgccacagaactacactaaagctaattgcacttattgtaacaccagggagtacacatttagttataaggggtgctgtttctactttactaagaaaaagcacacctggaatggatgcttccaggcctgtgctgaactgtatccatgcacatacttttatggcccaactcccgacatcctgcccgtggtgaccaggaacctgaatgccattgagtccctgtgggtgggagtgtacagggtgggagaaggcaactggacctccctggatggcgggacattcaaagtgtaccagatttttggctctcattgcacttatgtgtctaagttcagtaccgtgcccgtgtcacaccatgagtgtagctttctgaagccttgcctgtgtgtgtctcagagaagcaactcc

SEQ ID NO: 68 - protein, 223 aa, Epstein Barr Virus
MVSFKQVRVPLFTAIALVIVLLLAYFLPPRVRGGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 69 - DNA, 669 bp, Epstein Barr Virus
  source 1..669: mol_type = other DNA
ggagttgcttctctgagacacacacaggcaaggcttcagaaagctacactcatggtgtgacacgggcacggtactgaacttagacacataagtgcaatgagagccaaaaatctggtacactttgaatgtcccgccatccagggaggtccagttgccttctcccaccctgtacactcccacccacagggactcaatggcattcaggttcctggtcaccacgggcaggatgtcgggagttgggccataaaagtatgtgcatggatacagttcagcacaggcctggaagcatccattccaggtgtgctttttcttagtaaagtagaaacagcaccccttataactaaatgtgtactccctggtgttacaataagtgcaattagctttagtgtagttctgtggcacctggaatgtatgcagtgttggggtccagtgaggcagcttcacttctttatcgccatattcctgctcggctgtcttattaaagttcactggaggtggtgggtccacaggccacacttccacattaggttttggcacccaggtgatagcggcagcggccactcttcctcctcctctgacccgaggtggcaggaagtaggccagcagcagcacaatcaccagagcgatggcagtaaacaggggcacccgcacttgtttgaagctgaccat

SEQ ID NO: 70 - DNA, 3093 bp, synthetic construct
  misc_feature 1..3093: note = Synthetic
  source 1..3093: mol_type = other DNA
gaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgagtccactaccacatctcctaccctgaacactaccgggtttgccgaccccaatacaactaccggactgcctagctccacccatgtgccaacaaacctgactgcaccagcatccaccggacctacagtgtctactgccgatgtcaccagtcccacacctgccggaacaacttctggcgctagtcccgtgaccccatcacccagcccttgggacaatgggacagagagtaaggcccctgatatgacttctagtacctcaccagtcaccacaccaacccccaacgcaacaagccctactccagccgtgactacccccacacctaatgctaccagcccaacacccgcagtgacaactcctaccccaaacgccacttccccaaccctggggaagacatcacccactagcgccgtgaccacacccacccctaatgctacctctcctacactgggaaaaacttccccaacctctgcagtgactaccccaacccccaacgccacaagccccactctgggcaagaccagtcctacatcagctgtcacaactcctaccccaaatgcaactgggccaaccgtgggagagacatccccccaggctaacgcaacaaatcacactctgggaggcaccagtcccacacctgtggtcacctcacagcccaagaacgccacaagcgctgtgaccacaggccagcataatatcacatcaagctccacttctagtatgagcctgcgcccttcaagcaacccagagacactgtccccatctactagtgacaattcaaccagccacatgcctctgctgacatctgcacatccaactgggggagaaaacatcactcaggtcacccccgcctccatttctacccaccatgtgtccacatcctctccagcaccccgacctggaactaccagccaggcatccggaccaggaaatagttcaaccagcacaaagcctggcgaggtgaacgtcacaaaagggactccccctcagaatgctacctcacctcaggcaccaagcggccagaaaacagctgtgcctactgtcacctccacaggcgggaaggcaaactctacaactggaggcaaacacaccacagggcatggagctcgcactagcaccgaaccaactaccgactacgggggagattccacaactccaaggcccagatacaatgccaccacatatctgccaccctctaccagctccaagctgcgacccagatggacattcactagtcctccagtgactaccgcacaggctacagtgccagtcccacctacttctcagcctagattttctaacctgagttccggagagagccaggtgaggcagaacttcaagcccgagatggaggagaagctgaacgagcagatgaacctggagctgtacagcagcctgctgtaccagcagatgagcgcctggtgcagctaccacaccttcgagggcgccgccgccttcctgaggaggcacgcccaggaggagatgacccacatgcagaggctgttcgactacctgaccgacaccggcaacctgcccaggatcaacaccgtggagagccccttcgccgagtacagcagcctggacgagctgttccaggagacctacaagcacgagcagctgatcacccagaagatcaacgagctggcccacgccgccatgaccaaccaggactaccccaccttcaacttcctgcagtggtacgtgagcgagcagcacgaggaggagaagctgttcaagagcatcatcgacaagctgagcctggccggcaagagcggcgagggcctgtacttcatcgacaaggagctgagcaccctggacggatcc

SEQ ID NO: 71 - protein, 1031 aa, synthetic construct
  REGION 1..1031: note = Synthetic
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSSGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 72 - DNA, 3093 bp, synthetic construct
  misc_feature 1..3093: note = Synthetic
  source 1..3093: mol_type = other DNA
ggatccgtccagggtgctcagctccttgtcgatgaagtacaggccctcgccgctcttgccggccaggctcagcttgtcgatgatgctcttgaacagcttctcctcctcgtgctgctcgctcacgtaccactgcaggaagttgaaggtggggtagtcctggttggtcatggcggcgtgggccagctcgttgatcttctgggtgatcagctgctcgtgcttgtaggtctcctggaacagctcgtccaggctgctgtactcggcgaaggggctctccacggtgttgatcctgggcaggttgccggtgtcggtcaggtagtcgaacagcctctgcatgtgggtcatctcctcctgggcgtgcctcctcaggaaggcggcggcgccctcgaaggtgtggtagctgcaccaggcgctcatctgctggtacagcaggctgctgtacagctccaggttcatctgctcgttcagcttctcctccatctcgggcttgaagttctgcctcacctggctctctccggaactcaggttagaaaatctaggctgagaagtaggtgggactggcactgtagcctgtgcggtagtcactggaggactagtgaatgtccatctgggtcgcagcttggagctggtagagggtggcagatatgtggtggcattgtatctgggccttggagttgtggaatctcccccgtagtcggtagttggttcggtgctagtgcgagctccatgccctgtggtgtgtttgcctccagttgtagagtttgccttcccgcctgtggaggtgacagtaggcacagctgttttctggccgcttggtgcctgaggtgaggtagcattctgagggggagtcccttttgtgacgttcacctcgccaggctttgtgctggttgaactatttcctggtccggatgcctggctggtagttccaggtcggggtgctggagaggatgtggacacatggtgggtagaaatggaggcgggggtgacctgagtgatgttttctcccccagttggatgtgcagatgtcagcagaggcatgtggctggttgaattgtcactagtagatggggacagtgtctctgggttgcttgaagggcgcaggctcatactagaagtggagcttgatgtgatattatgctggcctgtggtcacagcgcttgtggcgttcttgggctgtgaggtgaccacaggtgtgggactggtgcctcccagagtgtgatttgttgcgttagcctggggggatgtctctcccacggttggcccagttgcatttggggtaggagttgtgacagctgatgtaggactggtcttgcccagagtggggcttgtggcgttgggggttggggtagtcactgcagaggttggggaagtttttcccagtgtaggagaggtagcattaggggtgggtgtggtcacggcgctagtgggtgatgtcttccccagggttggggaagtggcgtttggggtaggagttgtcactgcgggtgttgggctggtagcattaggtgtgggggtagtcacggctggagtagggcttgttgcgttgggggttggtgtggtgactggtgaggtactagaagtcatatcaggggccttactctctgtcccattgtcccaagggctgggtgatggggtcacgggactagcgccagaagttgttccggcaggtgtgggactggtgacatcggcagtagacactgtaggtccggtggatgctggtgcagtcaggtttgttggcacatgggtggagctaggcagtccggtagttgtattggggtcggcaaacccggtagtgttcagggtaggagatgtggtagtggactcgggagctttgctgaagatcactttgtgtgtggtagttgttgcgttagtagctgtccgggtaatgatcagggtttttggggcagtccccaggccggacactgtaatatcaaaggttcgattactagcgaaggcgccagagatgttttcacacccgctaggggtgcctgaagtcagtgtccatttgcacttaaaatcggtctcagtattgttgggccaggcccagaatgctgtgacggtcacatttggggagttagcgtcttcgcttgtgaccataggcacggaataagtggcattatcgcccacgtaggtaatgtcggtagtgttagtgggcatatcctggctggctgggatctcgtctgagaacacaatattactctggatgcaatagtcccctccagaagcctttggtccgtttccggaataaaacacgtacagaatgctattgttgcccaggaagcgtgagactggcctaggggtcagtctcaggctataagcgtagccggttccgggaattggagtggccacgggactagtagatgtcaggattcccccacttgggacgtgagattcgtagccggaacaggtgatattaaacttgttatcgccaggcagcacctggctaatttctccatcctccatgatgcattcaatgtcgatctcatttcccagcatctcggttttcacggagaagttgctatcctgggcgcttgtgggcagtgacagtggcagggtcacgtccagcccctgtgcccggaccactgcggtaatgttagtgctattacagttgtcccacttaatataagggacggtttcggggatcaggtacacaggattctgcatctcggcatggtggcaccacatggtgccaaacacatcctggaagtagacgtccactgattccagactgacctgctgttcctctcctgtggtcacattgatgggcagtttcttggacctcatggtcagagccagttctcctgctcccagcagctccagcagaaacagatttgttgcgttctcgctgcctccgaaagcccctctgggctgatacacggcttttgtatgtggggtcagctggccaaaatccaggtccagctggtgtttcttcccgcccacatcgaaattaatggtgacgttcacgtcggctgtacagacgttgcaggtggggtaaaatgggaactcagggatttccacattgaaaaatccagggtcctccccggtcagatgaatcaggctctgaatagtgtactggcacaccagcagggcggcttc

SEQ ID NO: 73 - DNA, 1923 bp, synthetic construct
  misc_feature 1..1923: note = Synthetic
  source 1..1923: mol_type = other DNA
gaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgagtccactaccacatctcctaccctgaacactaccgggtttgccgaccccaatacaactaccggactgcctagctccacccatgtgccaacaaacctgactgcaccagcatccaccggacctacagtgtctacttccggagagagccaggtgaggcagaacttcaagcccgagatggaggagaagctgaacgagcagatgaacctggagctgtacagcagcctgctgtaccagcagatgagcgcctggtgcagctaccacaccttcgagggcgccgccgccttcctgaggaggcacgcccaggaggagatgacccacatgcagaggctgttcgactacctgaccgacaccggcaacctgcccaggatcaacaccgtggagagccccttcgccgagtacagcagcctggacgagctgttccaggagacctacaagcacgagcagctgatcacccagaagatcaacgagctggcccacgccgccatgaccaaccaggactaccccaccttcaacttcctgcagtggtacgtgagcgagcagcacgaggaggagaagctgttcaagagcatcatcgacaagctgagcctggccggcaagagcggcgagggcctgtacttcatcgacaaggagctgagcaccctggacggatcc

SEQ ID NO: 74 - protein, 641 aa, synthetic construct
  REGION 1..641: note = Synthetic
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTSGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 75 - DNA, 1923 bp, synthetic construct
  misc_feature 1..1923: note = Synthetic
  source 1..1923: mol_type = other DNA
ggatccgtccagggtgctcagctccttgtcgatgaagtacaggccctcgccgctcttgccggccaggctcagcttgtcgatgatgctcttgaacagcttctcctcctcgtgctgctcgctcacgtaccactgcaggaagttgaaggtggggtagtcctggttggtcatggcggcgtgggccagctcgttgatcttctgggtgatcagctgctcgtgcttgtaggtctcctggaacagctcgtccaggctgctgtactcggcgaaggggctctccacggtgttgatcctgggcaggttgccggtgtcggtcaggtagtcgaacagcctctgcatgtgggtcatctcctcctgggcgtgcctcctcaggaaggcggcggcgccctcgaaggtgtggtagctgcaccaggcgctcatctgctggtacagcaggctgctgtacagctccaggttcatctgctcgttcagcttctcctccatctcgggcttgaagttctgcctcacctggctctctccggaagtagacactgtaggtccggtggatgctggtgcagtcaggtttgttggcacatgggtggagctaggcagtccggtagttgtattggggtcggcaaacccggtagtgttcagggtaggagatgtggtagtggactcgggagctttgctgaagatcactttgtgtgtggtagttgttgcgttagtagctgtccgggtaatgatcagggtttttggggcagtccccaggccggacactgtaatatcaaaggttcgattactagcgaaggcgccagagatgttttcacacccgctaggggtgcctgaagtcagtgtccatttgcacttaaaatcggtctcagtattgttgggccaggcccagaatgctgtgacggtcacatttggggagttagcgtcttcgcttgtgaccataggcacggaataagtggcattatcgcccacgtaggtaatgtcggtagtgttagtgggcatatcctggctggctgggatctcgtctgagaacacaatattactctggatgcaatagtcccctccagaagcctttggtccgtttccggaataaaacacgtacagaatgctattgttgcccaggaagcgtgagactggcctaggggtcagtctcaggctataagcgtagccggttccgggaattggagtggccacgggactagtagatgtcaggattcccccacttgggacgtgagattcgtagccggaacaggtgatattaaacttgttatcgccaggcagcacctggctaatttctccatcctccatgatgcattcaatgtcgatctcatttcccagcatctcggttttcacggagaagttgctatcctgggcgcttgtgggcagtgacagtggcagggtcacgtccagcccctgtgcccggaccactgcggtaatgttagtgctattacagttgtcccacttaatataagggacggtttcggggatcaggtacacaggattctgcatctcggcatggtggcaccacatggtgccaaacacatcctggaagtagacgtccactgattccagactgacctgctgttcctctcctgtggtcacattgatgggcagtttcttggacctcatggtcagagccagttctcctgctcccagcagctccagcagaaacagatttgttgcgttctcgctgcctccgaaagcccctctgggctgatacacggcttttgtatgtggggtcagctggccaaaatccaggtccagctggtgtttcttcccgcccacatcgaaattaatggtgacgttcacgtcggctgtacagacgttgcaggtggggtaaaatgggaactcagggatttccacattgaaaaatccagggtcctccccggtcagatgaatcaggctctgaatagtgtactggcacaccagcagggcggcttc

SEQ ID NO: 76 - DNA, 1464 bp, synthetic construct
  misc_feature 1..1464: note = Synthetic
  source 1..1464: mol_type = other DNA
gaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccacttccggagagagccaggtgaggcagaacttcaagcccgagatggaggagaagctgaacgagcagatgaacctggagctgtacagcagcctgctgtaccagcagatgagcgcctggtgcagctaccacaccttcgagggcgccgccgccttcctgaggaggcacgcccaggaggagatgacccacatgcagaggctgttcgactacctgaccgacaccggcaacctgcccaggatcaacaccgtggagagccccttcgccgagtacagcagcctggacgagctgttccaggagacctacaagcacgagcagctgatcacccagaagatcaacgagctggcccacgccgccatgaccaaccaggactaccccaccttcaacttcctgcagtggtacgtgagcgagcagcacgaggaggagaagctgttcaagagcatcatcgacaagctgagcctggccggcaagagcggcgagggcctgtacttcatcgacaaggagctgagcaccctggacggatcc

SEQ ID NO: 77 - protein, 488 aa, synthetic construct
  REGION 1..488: note = Synthetic
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTSGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 78 - DNA, 1464 bp, synthetic construct
  misc_feature 1..1464: note = Synthetic
  source 1..1464: mol_type = other DNA
ggatccgtccagggtgctcagctccttgtcgatgaagtacaggccctcgccgctcttgccggccaggctcagcttgtcgatgatgctcttgaacagcttctcctcctcgtgctgctcgctcacgtaccactgcaggaagttgaaggtggggtagtcctggttggtcatggcggcgtgggccagctcgttgatcttctgggtgatcagctgctcgtgcttgtaggtctcctggaacagctcgtccaggctgctgtactcggcgaaggggctctccacggtgttgatcctgggcaggttgccggtgtcggtcaggtagtcgaacagcctctgcatgtgggtcatctcctcctgggcgtgcctcctcaggaaggcggcggcgccctcgaaggtgtggtagctgcaccaggcgctcatctgctggtacagcaggctgctgtacagctccaggttcatctgctcgttcagcttctcctccatctcgggcttgaagttctgcctcacctggctctctccggaagtgggcatatcctggctggctgggatctcgtctgagaacacaatattactctggatgcaatagtcccctccagaagcctttggtccgtttccggaataaaacacgtacagaatgctattgttgcccaggaagcgtgagactggcctaggggtcagtctcaggctataagcgtagccggttccgggaattggagtggccacgggactagtagatgtcaggattcccccacttgggacgtgagattcgtagccggaacaggtgatattaaacttgttatcgccaggcagcacctggctaatttctccatcctccatgatgcattcaatgtcgatctcatttcccagcatctcggttttcacggagaagttgctatcctgggcgcttgtgggcagtgacagtggcagggtcacgtccagcccctgtgcccggaccactgcggtaatgttagtgctattacagttgtcccacttaatataagggacggtttcggggatcaggtacacaggattctgcatctcggcatggtggcaccacatggtgccaaacacatcctggaagtagacgtccactgattccagactgacctgctgttcctctcctgtggtcacattgatgggcagtttcttggacctcatggtcagagccagttctcctgctcccagcagctccagcagaaacagatttgttgcgttctcgctgcctccgaaagcccctctgggctgatacacggcttttgtatgtggggtcagctggccaaaatccaggtccagctggtgtttcttcccgcccacatcgaaattaatggtgacgttcacgtcggctgtacagacgttgcaggtggggtaaaatgggaactcagggatttccacattgaaaaatccagggtcctccccggtcagatgaatcaggctctgaatagtgtactggcacaccagcagggcggcttc

SEQ ID NO: 79 - DNA, 1788 bp, synthetic construct
  misc_feature 1..1788: note = Synthetic
  source 1..1788: mol_type = other DNA
gaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctccctccggagagagccaggtgaggcagaacttcaagcccgagatggaggagaagctgaacgagcagatgaacctggagctgtacagcagcctgctgtaccagcagatgagcgcctggtgcagctaccacaccttcgagggcgccgccgccttcctgaggaggcacgcccaggaggagatgacccacatgcagaggctgttcgactacctgaccgacaccggcaacctgcccaggatcaacaccgtggagagccccttcgccgagtacagcagcctggacgagctgttccaggagacctacaagcacgagcagctgatcacccagaagatcaacgagctggcccacgccgccatgaccaaccaggactaccccaccttcaacttcctgcagtggtacgtgagcgagcagcacgaggaggagaagctgttcaagagcatcatcgacaagctgagcctggccggcaagagcggcgagggcctgtacttcatcgacaaggagctgagcaccctggacggatcc

SEQ ID NO: 80 - protein, 596 aa, synthetic construct
  REGION 1..596: note = Synthetic
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPSGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 81 - DNA, 1788 bp, synthetic construct
  misc_feature 1..1788: note = Synthetic
  source 1..1788: mol_type = other DNA
ggatccgtccagggtgctcagctccttgtcgatgaagtacaggccctcgccgctcttgccggccaggctcagcttgtcgatgatgctcttgaacagcttctcctcctcgtgctgctcgctcacgtaccactgcaggaagttgaaggtggggtagtcctggttggtcatggcggcgtgggccagctcgttgatcttctgggtgatcagctgctcgtgcttgtaggtctcctggaacagctcgtccaggctgctgtactcggcgaaggggctctccacggtgttgatcctgggcaggttgccggtgtcggtcaggtagtcgaacagcctctgcatgtgggtcatctcctcctgggcgtgcctcctcaggaaggcggcggcgccctcgaaggtgtggtagctgcaccaggcgctcatctgctggtacagcaggctgctgtacagctccaggttcatctgctcgttcagcttctcctccatctcgggcttgaagttctgcctcacctggctctctccggagggagctttgctgaagatcactttgtgtgtggtagttgttgcgttagtagctgtccgggtaatgatcagggtttttggggcagtccccaggccggacactgtaatatcaaaggttcgattactagcgaaggcgccagagatgttttcacacccgctaggggtgcctgaagtcagtgtccatttgcacttaaaatcggtctcagtattgttgggccaggcccagaatgctgtgacggtcacatttggggagttagcgtcttcgcttgtgaccataggcacggaataagtggcattatcgcccacgtaggtaatgtcggtagtgttagtgggcatatcctggctggctgggatctcgtctgagaacacaatattactctggatgcaatagtcccctccagaagcctttggtccgtttccggaataaaacacgtacagaatgctattgttgcccaggaagcgtgagactggcctaggggtcagtctcaggctataagcgtagccggttccgggaattggagtggccacgggactagtagatgtcaggattcccccacttgggacgtgagattcgtagccggaacaggtgatattaaacttgttatcgccaggcagcacctggctaatttctccatcctccatgatgcattcaatgtcgatctcatttcccagcatctcggttttcacggagaagttgctatcctgggcgcttgtgggcagtgacagtggcagggtcacgtccagcccctgtgcccggaccactgcggtaatgttagtgctattacagttgtcccacttaatataagggacggtttcggggatcaggtacacaggattctgcatctcggcatggtggcaccacatggtgccaaacacatcctggaagtagacgtccactgattccagactgacctgctgttcctctcctgtggtcacattgatgggcagtttcttggacctcatggtcagagccagttctcctgctcccagcagctccagcagaaacagatttgttgcgttctcgctgcctccgaaagcccctctgggctgatacacggcttttgtatgtggggtcagctggccaaaatccaggtccagctggtgtttcttcccgcccacatcgaaattaatggtgacgttcacgtcggctgtacagacgttgcaggtggggtaaaatgggaactcagggatttccacattgaaaaatccagggtcctccccggtcagatgaatcaggctctgaatagtgtactggcacaccagcagggcggcttc

SEQ ID NO: 82 - DNA, 3108 bp, synthetic construct
  misc_feature 1..3108: note = Synthetic
  source 1..3108: mol_type = other DNA
gaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgagtccactaccacatctcctaccctgaacactaccgggtttgccgaccccaatacaactaccggactgcctagctccacccatgtgccaacaaacctgactgcaccagcatccaccggacctacagtgtctactgccgatgtcaccagtcccacacctgccggaacaacttctggcgctagtcccgtgaccccatcacccagcccttgggacaatgggacagagagtaaggcccctgatatgacttctagtacctcaccagtcaccacaccaacccccaacgcaacaagccctactccagccgtgactacccccacacctaatgctaccagcccaacacccgcagtgacaactcctaccccaaacgccacttccccaaccctggggaagacatcacccactagcgccgtgaccacacccacccctaatgctacctctcctacactgggaaaaacttccccaacctctgcagtgactaccccaacccccaacgccacaagccccactctgggcaagaccagtcctacatcagctgtcacaactcctaccccaaatgcaactgggccaaccgtgggagagacatccccccaggctaacgcaacaaatcacactctgggaggcaccagtcccacacctgtggtcacctcacagcccaagaacgccacaagcgctgtgaccacaggccagcataatatcacatcaagctccacttctagtatgagcctgcgcccttcaagcaacccagagacactgtccccatctactagtgacaattcaaccagccacatgcctctgctgacatctgcacatccaactgggggagaaaacatcactcaggtcacccccgcctccatttctacccaccatgtgtccacatcctctccagcaccccgacctggaactaccagccaggcatccggaccaggaaatagttcaaccagcacaaagcctggcgaggtgaacgtcacaaaagggactccccctcagaatgctacctcacctcaggcaccaagcggccagaaaacagctgtgcctactgtcacctccacaggcgggaaggcaaactctacaactggaggcaaacacaccacagggcatggagctcgcactagcaccgaaccaactaccgactacgggggagattccacaactccaaggcccagatacaatgccaccacatatctgccaccctctaccagctccaagctgcgacccagatggacattcactagtcctccagtgactaccgcacaggctacagtgccagtcccacctacttctcagcctagattttctaacctgagttccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcc

SEQ ID NO: 83 - protein, 1036 aa, synthetic construct
  REGION 1..1036: note = Synthetic
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 84 - DNA, 3108 bp, synthetic construct
  misc_feature 1..3108: note = Synthetic
  source 1..3108: mol_type = other DNA
ggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaactcaggttagaaaatctaggctgagaagtaggtgggactggcactgtagcctgtgcggtagtcactggaggactagtgaatgtccatctgggtcgcagcttggagctggtagagggtggcagatatgtggtggcattgtatctgggccttggagttgtggaatctcccccgtagtcggtagttggttcggtgctagtgcgagctccatgccctgtggtgtgtttgcctccagttgtagagtttgccttcccgcctgtggaggtgacagtaggcacagctgttttctggccgcttggtgcctgaggtgaggtagcattctgagggggagtcccttttgtgacgttcacctcgccaggctttgtgctggttgaactatttcctggtccggatgcctggctggtagttccaggtcggggtgctggagaggatgtggacacatggtgggtagaaatggaggcgggggtgacctgagtgatgttttctcccccagttggatgtgcagatgtcagcagaggcatgtggctggttgaattgtcactagtagatggggacagtgtctctgggttgcttgaagggcgcaggctcatactagaagtggagcttgatgtgatattatgctggcctgtggtcacagcgcttgtggcgttcttgggctgtgaggtgaccacaggtgtgggactggtgcctcccagagtgtgatttgttgcgttagcctggggggatgtctctcccacggttggcccagttgcatttggggtaggagttgtgacagctgatgtaggactggtcttgcccagagtggggcttgtggcgttgggggttggggtagtcactgcagaggttggggaagtttttcccagtgtaggagaggtagcattaggggtgggtgtggtcacggcgctagtgggtgatgtcttccccagggttggggaagtggcgtttggggtaggagttgtcactgcgggtgttgggctggtagcattaggtgtgggggtagtcacggctggagtagggcttgttgcgttgggggttggtgtggtgactggtgaggtactagaagtcatatcaggggccttactctctgtcccattgtcccaagggctgggtgatggggtcacgggactagcgccagaagttgttccggcaggtgtgggactggtgacatcggcagtagacactgtaggtccggtggatgctggtgcagtcaggtttgttggcacatgggtggagctaggcagtccggtagttgtattggggtcggcaaacccggtagtgttcagggtaggagatgtggtagtggactcgggagctttgctgaagatcactttgtgtgtggtagttgttgcgttagtagctgtccgggtaatgatcagggtttttggggcagtccccaggccggacactgtaatatcaaaggttcgattactagcgaaggcgccagagatgttttcacacccgctaggggtgcctgaagtcagtgtccatttgcacttaaaatcggtctcagtattgttgggccaggcccagaatgctgtgacggtcacatttggggagttagcgtcttcgcttgtgaccataggcacggaataagtggcattatcgcccacgtaggtaatgtcggtagtgttagtgggcatatcctggctggctgggatctcgtctgagaacacaatattactctggatgcaatagtcccctccagaagcctttggtccgtttccggaataaaacacgtacagaatgctattgttgcccaggaagcgtgagactggcctaggggtcagtctcaggctataagcgtagccggttccgggaattggagtggccacgggactagtagatgtcaggattcccccacttgggacgtgagattcgtagccggaacaggtgatattaaacttgttatcgccaggcagcacctggctaatttctccatcctccatgatgcattcaatgtcgatctcatttcccagcatctcggttttcacggagaagttgctatcctgggcgcttgtgggcagtgacagtggcagggtcacgtccagcccctgtgcccggaccactgcggtaatgttagtgctattacagttgtcccacttaatataagggacggtttcggggatcaggtacacaggattctgcatctcggcatggtggcaccacatggtgccaaacacatcctggaagtagacgtccactgattccagactgacctgctgttcctctcctgtggtcacattgatgggcagtttcttggacctcatggtcagagccagttctcctgctcccagcagctccagcagaaacagatttgttgcgttctcgctgcctccgaaagcccctctgggctgatacacggcttttgtatgtggggtcagctggccaaaatccaggtccagctggtgtttcttcccgcccacatcgaaattaatggtgacgttcacgtcggctgtacagacgttgcaggtggggtaaaatgggaactcagggatttccacattgaaaaatccagggtcctccccggtcagatgaatcaggctctgaatagtgtactggcacaccagcagggcggcttc

SEQ ID NO: 85 - DNA, 1479 bp, synthetic construct
  misc_feature 1..1479: note = Synthetic
  source 1..1479: mol_type = other DNA
gaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccacttccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcc

SEQ ID NO: 86 - protein, 493 aa, synthetic construct
  REGION 1..493: note = Synthetic
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 87 - DNA, 1479 bp, synthetic construct
  misc_feature 1..1479: note = Synthetic
  source 1..1479: mol_type = other DNA
ggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaagtgggcatatcctggctggctgggatctcgtctgagaacacaatattactctggatgcaatagtcccctccagaagcctttggtccgtttccggaataaaacacgtacagaatgctattgttgcccaggaagcgtgagactggcctaggggtcagtctcaggctataagcgtagccggttccgggaattggagtggccacgggactagtagatgtcaggattcccccacttgggacgtgagattcgtagccggaacaggtgatattaaacttgttatcgccaggcagcacctggctaatttctccatcctccatgatgcattcaatgtcgatctcatttcccagcatctcggttttcacggagaagttgctatcctgggcgcttgtgggcagtgacagtggcagggtcacgtccagcccctgtgcccggaccactgcggtaatgttagtgctattacagttgtcccacttaatataagggacggtttcggggatcaggtacacaggattctgcatctcggcatggtggcaccacatggtgccaaacacatcctggaagtagacgtccactgattccagactgacctgctgttcctctcctgtggtcacattgatgggcagtttcttggacctcatggtcagagccagttctcctgctcccagcagctccagcagaaacagatttgttgcgttctcgctgcctccgaaagcccctctgggctgatacacggcttttgtatgtggggtcagctggccaaaatccaggtccagctggtgtttcttcccgcccacatcgaaattaatggtgacgttcacgtcggctgtacagacgttgcaggtggggtaaaatgggaactcagggatttccacattgaaaaatccagggtcctccccggtcagatgaatcaggctctgaatagtgtactggcacaccagcagggcggcttc

SEQ ID NO: 88 - DNA, 1803 bp, synthetic construct
  misc_feature 1..1803: note = Synthetic
  source 1..1803: mol_type = other DNA
gaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctccctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcc

SEQ ID NO: 89 - protein, 601 aa, synthetic construct
  REGION 1..601: note = Synthetic
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 90 - DNA, 1803 bp, synthetic construct
  misc_feature 1..1803: note = Synthetic
  source 1..1803: mol_type = other DNA
ggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggagggagctttgctgaagatcactttgtgtgtggtagttgttgcgttagtagctgtccgggtaatgatcagggtttttggggcagtccccaggccggacactgtaatatcaaaggttcgattactagcgaaggcgccagagatgttttcacacccgctaggggtgcctgaagtcagtgtccatttgcacttaaaatcggtctcagtattgttgggccaggcccagaatgctgtgacggtcacatttggggagttagcgtcttcgcttgtgaccataggcacggaataagtggcattatcgcccacgtaggtaatgtcggtagtgttagtgggcatatcctggctggctgggatctcgtctgagaacacaatattactctggatgcaatagtcccctccagaagcctttggtccgtttccggaataaaacacgtacagaatgctattgttgcccaggaagcgtgagactggcctaggggtcagtctcaggctataagcgtagccggttccgggaattggagtggccacgggactagtagatgtcaggattcccccacttgggacgtgagattcgtagccggaacaggtgatattaaacttgttatcgccaggcagcacctggctaatttctccatcctccatgatgcattcaatgtcgatctcatttcccagcatctcggttttcacggagaagttgctatcctgggcgcttgtgggcagtgacagtggcagggtcacgtccagcccctgtgcccggaccactgcggtaatgttagtgctattacagttgtcccacttaatataagggacggtttcggggatcaggtacacaggattctgcatctcggcatggtggcaccacatggtgccaaacacatcctggaagtagacgtccactgattccagactgacctgctgttcctctcctgtggtcacattgatgggcagtttcttggacctcatggtcagagccagttctcctgctcccagcagctccagcagaaacagatttgttgcgttctcgctgcctccgaaagcccctctgggctgatacacggcttttgtatgtggggtcagctggccaaaatccaggtccagctggtgtttcttcccgcccacatcgaaattaatggtgacgttcacgtcggctgtacagacgttgcaggtggggtaaaatgggaactcagggatttccacattgaaaaatccagggtcctccccggtcagatgaatcaggctctgaatagtgtactggcacaccagcagggcggcttc

SEQ ID NO: 91 - DNA, 1938 bp, synthetic construct
  misc_feature 1..1938: note = Synthetic
  source 1..1938: mol_type = other DNA
gaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgagtccactaccacatctcctaccctgaacactaccgggtttgccgaccccaatacaactaccggactgcctagctccacccatgtgccaacaaacctgactgcaccagcatccaccggacctacagtgtctacttccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcc

SEQ ID NO: 92 - protein, 646 aa, synthetic construct
  REGION 1..646: note = Synthetic
EAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 93 - DNA, 1938 bp, synthetic construct
  misc_feature 1..1938: note = Synthetic
  source 1..1938: mol_type = other DNA
ggatccgctcttcctgctcttggcgatgcccttcacgtactggtcggccaggtacaggccgtggttctcgttgccgatcagctcgatcttgtccaggatgtccttgaacagcacctcctcctcgtgctgctcggccacgtaccactgcaggaagttgaaggtggcgtggtccttgctcttgatggcgtggtccacgatgttgttgatgctctcgctgatgtgctgctcgtgctcgtaggccttctggaagatctgggtcaggccctcgaacttgtgctcgggggcgctgatgctggtcagctgcacgggcacgttgttctcgttcaggaagatgatcagcttcttggcgtgctcgtactcctcggcggcgtggtcgaacaggaacaggccggcgccgtccaggctgtgggtgtagcaccagctgctcatgctcatgtacaggttgctgctctgcatctccttgttcacctgctcgttcagcagcttctcgatgtccttgctgaactgctgcctcacctggctctctccggaagtagacactgtaggtccggtggatgctggtgcagtcaggtttgttggcacatgggtggagctaggcagtccggtagttgtattggggtcggcaaacccggtagtgttcagggtaggagatgtggtagtggactcgggagctttgctgaagatcactttgtgtgtggtagttgttgcgttagtagctgtccgggtaatgatcagggtttttggggcagtccccaggccggacactgtaatatcaaaggttcgattactagcgaaggcgccagagatgttttcacacccgctaggggtgcctgaagtcagtgtccatttgcacttaaaatcggtctcagtattgttgggccaggcccagaatgctgtgacggtcacatttggggagttagcgtcttcgcttgtgaccataggcacggaataagtggcattatcgcccacgtaggtaatgtcggtagtgttagtgggcatatcctggctggctgggatctcgtctgagaacacaatattactctggatgcaatagtcccctccagaagcctttggtccgtttccggaataaaacacgtacagaatgctattgttgcccaggaagcgtgagactggcctaggggtcagtctcaggctataagcgtagccggttccgggaattggagtggccacgggactagtagatgtcaggattcccccacttgggacgtgagattcgtagccggaacaggtgatattaaacttgttatcgccaggcagcacctggctaatttctccatcctccatgatgcattcaatgtcgatctcatttcccagcatctcggttttcacggagaagttgctatcctgggcgcttgtgggcagtgacagtggcagggtcacgtccagcccctgtgcccggaccactgcggtaatgttagtgctattacagttgtcccacttaatataagggacggtttcggggatcaggtacacaggattctgcatctcggcatggtggcaccacatggtgccaaacacatcctggaagtagacgtccactgattccagactgacctgctgttcctctcctgtggtcacattgatgggcagtttcttggacctcatggtcagagccagttctcctgctcccagcagctccagcagaaacagatttgttgcgttctcgctgcctccgaaagcccctctgggctgatacacggcttttgtatgtggggtcagctggccaaaatccaggtccagctggtgtttcttcccgcccacatcgaaattaatggtgacgttcacgtcggctgtacagacgttgcaggtggggtaaaatgggaactcagggatttccacattgaaaaatccagggtcctccccggtcagatgaatcaggctctgaatagtgtactggcacaccagcagggcggcttc

SEQ ID NO: 94 - DNA, 3393 bp, synthetic construct
  misc_feature 1..3393: note = Synthetic
  source 1..3393: mol_type = other DNA
atggagttcctgaagaggagcttcgcccctctgaccgagaagcagtggcaggagatcgacaacagggccagggagatcttcaagacccagctgtacggcaggaagttcgtggacgtggagggcccctacggctgggagtacgccgcccaccccctgggcgaggtggaggtgctgagcgacgagaacgaggtggtgaagtggggcctgaggaagagcctgcccctgatcgagctgagggccaccttcaccctggacctgtgggagctggacaacctggagaggggcaagcccaacgtggacctgagcagcctggaggagaccgtgaggaaggtggccgagttcgaggacgaggtgatcttcaggggctgcgagaagagcggcgtgaagggcctgctgagcttcgaggagaggaagatcgagtgcggcagcacccccaaggacctgctggaggccatcgtgagggccctgagcatcttcagcaaggacggcatcgagggcccctacaccctggtgatcaacaccgacaggtggatcaacttcctgaaggaggaggccggccactaccccctggagaagagggtggaggagtgcctgaggggcggcaagatcatcaccacccccaggatcgaggacgccctggtggtgagcgagaggggcggcgacttcaagctgatcctgggccaggacctgagcatcggctacgaggacagggagaaggacgccgtgaggctgttcatcaccgagaccttcaccttccaggtggtgaaccccgaggccctgatcctgctgaagtccggaggcggatctggcggaggcgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgagtccactaccacatctcctaccctgaacactaccgggtttgccgaccccaatacaactaccggactgcctagctccacccatgtgccaacaaacctgactgcaccagcatccaccggacctacagtgtctactgccgatgtcaccagtcccacacctgccggaacaacttctggcgctagtcccgtgaccccatcacccagcccttgggacaatgggacagagagtaaggcccctgatatgacttctagtacctcaccagtcaccacaccaacccccaacgcaacaagccctactccagccgtgactacccccacacctaatgctaccagcccaacacccgcagtgacaactcctaccccaaacgccacttccccaaccctggggaagacatcacccactagcgccgtgaccacacccacccctaatgctacctctcctacactgggaaaaacttccccaacctctgcagtgactaccccaacccccaacgccacaagccccactctgggcaagaccagtcctacatcagctgtcacaactcctaccccaaatgcaactgggccaaccgtgggagagacatccccccaggctaacgcaacaaatcacactctgggaggcaccagtcccacacctgtggtcacctcacagcccaagaacgccacaagcgctgtgaccacaggccagcataatatcacatcaagctccacttctagtatgagcctgcgcccttcaagcaacccagagacactgtccccatctactagtgacaattcaaccagccacatgcctctgctgacatctgcacatccaactgggggagaaaacatcactcaggtcacccccgcctccatttctacccaccatgtgtccacatcctctccagcaccccgacctggaactaccagccaggcatccggaccaggaaatagttcaaccagcacaaagcctggcgaggtgaacgtcacaaaagggactccccctcagaatgctacctcacctcaggcaccaagcggccagaaaacagctgtgcctactgtcacctccacaggcgggaaggcaaactctacaactggaggcaaacacaccacagggcatggagctcgcactagcaccgaaccaactaccgactacgggggagattccacaactccaaggcccagatacaatgccaccacatatctgccaccctctaccagctccaagctgcgacccagatggacattcactagtcctccagtgactaccgcacaggctacagtgccagtcccacctacttctcagcctagattttctaacctgagt

SEQ ID NO: 95 - protein, 1131 aa, synthetic construct
  REGION 1..1131: note = Synthetic
MEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKSGGGSGGGEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLS

SEQ ID NO: 96 - DNA, 3393 bp, synthetic construct
  misc_feature 1..3393: note = Synthetic
  source 1..3393: mol_type = other DNA
actcaggttagaaaatctaggctgagaagtaggtgggactggcactgtagcctgtgcggtagtcactggaggactagtgaatgtccatctgggtcgcagcttggagctggtagagggtggcagatatgtggtggcattgtatctgggccttggagttgtggaatctcccccgtagtcggtagttggttcggtgctagtgcgagctccatgccctgtggtgtgtttgcctccagttgtagagtttgccttcccgcctgtggaggtgacagtaggcacagctgttttctggccgcttggtgcctgaggtgaggtagcattctgagggggagtcccttttgtgacgttcacctcgccaggctttgtgctggttgaactatttcctggtccggatgcctggctggtagttccaggtcggggtgctggagaggatgtggacacatggtgggtagaaatggaggcgggggtgacctgagtgatgttttctcccccagttggatgtgcagatgtcagcagaggcatgtggctggttgaattgtcactagtagatggggacagtgtctctgggttgcttgaagggcgcaggctcatactagaagtggagcttgatgtgatattatgctggcctgtggtcacagcgcttgtggcgttcttgggctgtgaggtgaccacaggtgtgggactggtgcctcccagagtgtgatttgttgcgttagcctggggggatgtctctcccacggttggcccagttgcatttggggtaggagttgtgacagctgatgtaggactggtcttgcccagagtggggcttgtggcgttgggggttggggtagtcactgcagaggttggggaagtttttcccagtgtaggagaggtagcattaggggtgggtgtggtcacggcgctagtgggtgatgtcttccccagggttggggaagtggcgtttggggtaggagttgtcactgcgggtgttgggctggtagcattaggtgtgggggtagtcacggctggagtagggcttgttgcgttgggggttggtgtggtgactggtgaggtactagaagtcatatcaggggccttactctctgtcccattgtcccaagggctgggtgatggggtcacgggactagcgccagaagttgttccggcaggtgtgggactggtgacatcggcagtagacactgtaggtccggtggatgctggtgcagtcaggtttgttggcacatgggtggagctaggcagtccggtagttgtattggggtcggcaaacccggtagtgttcagggtaggagatgtggtagtggactcgggagctttgctgaagatcactttgtgtgtggtagttgttgcgttagtagctgtccgggtaatgatcagggtttttggggcagtccccaggccggacactgtaatatcaaaggttcgattactagcgaaggcgccagagatgttttcacacccgctaggggtgcctgaagtcagtgtccatttgcacttaaaatcggtctcagtattgttgggccaggcccagaatgctgtgacggtcacatttggggagttagcgtcttcgcttgtgaccataggcacggaataagtggcattatcgcccacgtaggtaatgtcggtagtgttagtgggcatatcctggctggctgggatctcgtctgagaacacaatattactctggatgcaatagtcccctccagaagcctttggtccgtttccggaataaaacacgtacagaatgctattgttgcccaggaagcgtgagactggcctaggggtcagtctcaggctataagcgtagccggttccgggaattggagtggccacgggactagtagatgtcaggattcccccacttgggacgtgagattcgtagccggaacaggtgatattaaacttgttatcgccaggcagcacctggctaatttctccatcctccatgatgcattcaatgtcgatctcatttcccagcatctcggttttcacggagaagttgctatcctgggcgcttgtgggcagtgacagtggcagggtcacgtccagcccctgtgcccggaccactgcggtaatgttagtgctattacagttgtcccacttaatataagggacggtttcggggatcaggtacacaggattctgcatctcggcatggtggcaccacatggtgccaaacacatcctggaagtagacgtccactgattccagactgacctgctgttcctctcctgtggtcacattgatgggcagtttcttggacctcatggtcagagccagttctcctgctcccagcagctccagcagaaacagatttgttgcgttctcgctgcctccgaaagcccctctgggctgatacacggcttttgtatgtggggtcagctggccaaaatccaggtccagctggtgtttcttcccgcccacatcgaaattaatggtgacgttcacgtcggctgtacagacgttgcaggtggggtaaaatgggaactcagggatttccacattgaaaaatccagggtcctccccggtcagatgaatcaggctctgaatagtgtactggcacaccagcagggcggcttcgcctccgccagatccgcctccggacttcagcaggatcagggcctcggggttcaccacctggaaggtgaaggtctcggtgatgaacagcctcacggcgtccttctccctgtcctcgtagccgatgctcaggtcctggcccaggatcagcttgaagtcgccgcccctctcgctcaccaccagggcgtcctcgatcctgggggtggtgatgatcttgccgcccctcaggcactcctccaccctcttctccagggggtagtggccggcctcctccttcaggaagttgatccacctgtcggtgttgatcaccagggtgtaggggccctcgatgccgtccttgctgaagatgctcagggccctcacgatggcctccagcaggtccttgggggtgctgccgcactcgatcttcctctcctcgaagctcagcaggcccttcacgccgctcttctcgcagcccctgaagatcacctcgtcctcgaactcggccaccttcctcacggtctcctccaggctgctcaggtccacgttgggcttgcccctctccaggttgtccagctcccacaggtccagggtgaaggtggccctcagctcgatcaggggcaggctcttcctcaggccccacttcaccacctcgttctcgtcgctcagcacctccacctcgcccagggggtgggcggcgtactcccagccgtaggggccctccacgtccacgaacttcctgccgtacagctgggtcttgaagatctccctggccctgttgtcgatctcctgccactgcttctcggtcagaggggcgaagctcctcttcaggaactccat

SEQ ID NO: 97 - DNA, 2223 bp, synthetic construct
  misc_feature 1..2223: note = Synthetic
  source 1..2223: mol_type = other DNA
atggagttcctgaagaggagcttcgcccctctgaccgagaagcagtggcaggagatcgacaacagggccagggagatcttcaagacccagctgtacggcaggaagttcgtggacgtggagggcccctacggctgggagtacgccgcccaccccctgggcgaggtggaggtgctgagcgacgagaacgaggtggtgaagtggggcctgaggaagagcctgcccctgatcgagctgagggccaccttcaccctggacctgtgggagctggacaacctggagaggggcaagcccaacgtggacctgagcagcctggaggagaccgtgaggaaggtggccgagttcgaggacgaggtgatcttcaggggctgcgagaagagcggcgtgaagggcctgctgagcttcgaggagaggaagatcgagtgcggcagcacccccaaggacctgctggaggccatcgtgagggccctgagcatcttcagcaaggacggcatcgagggcccctacaccctggtgatcaacaccgacaggtggatcaacttcctgaaggaggaggccggccactaccccctggagaagagggtggaggagtgcctgaggggcggcaagatcatcaccacccccaggatcgaggacgccctggtggtgagcgagaggggcggcgacttcaagctgatcctgggccaggacctgagcatcggctacgaggacagggagaaggacgccgtgaggctgttcatcaccgagaccttcaccttccaggtggtgaaccccgaggccctgatcctgctgaagtccggaggcggatctggcggaggcgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgagtccactaccacatctcctaccctgaacactaccgggtttgccgaccccaatacaactaccggactgcctagctccacccatgtgccaacaaacctgactgcaccagcatccaccggacctacagtgtctact

SEQ ID NO: 98 - protein, 741 aa, synthetic construct
  REGION 1..741: note = Synthetic
MEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKSGGGSGGGEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVST

SEQ ID NO: 99 - skipped

SEQ ID NO: 100 - DNA, 1764 bp, synthetic construct
  misc_feature 1..1764: note = Synthetic
  source 1..1764: mol_type = other DNA
atggagttcctgaagaggagcttcgcccctctgaccgagaagcagtggcaggagatcgacaacagggccagggagatcttcaagacccagctgtacggcaggaagttcgtggacgtggagggcccctacggctgggagtacgccgcccaccccctgggcgaggtggaggtgctgagcgacgagaacgaggtggtgaagtggggcctgaggaagagcctgcccctgatcgagctgagggccaccttcaccctggacctgtgggagctggacaacctggagaggggcaagcccaacgtggacctgagcagcctggaggagaccgtgaggaaggtggccgagttcgaggacgaggtgatcttcaggggctgcgagaagagcggcgtgaagggcctgctgagcttcgaggagaggaagatcgagtgcggcagcacccccaaggacctgctggaggccatcgtgagggccctgagcatcttcagcaaggacggcatcgagggcccctacaccctggtgatcaacaccgacaggtggatcaacttcctgaaggaggaggccggccactaccccctggagaagagggtggaggagtgcctgaggggcggcaagatcatcaccacccccaggatcgaggacgccctggtggtgagcgagaggggcggcgacttcaagctgatcctgggccaggacctgagcatcggctacgaggacagggagaaggacgccgtgaggctgttcatcaccgagaccttcaccttccaggtggtgaaccccgaggccctgatcctgctgaagtccggaggcggatctggcggaggcgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccact

SEQ ID NO: 101 - protein, 588 aa, synthetic construct
  REGION 1..588: note = Synthetic
MEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKSGGGSGGGEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPT

SEQ ID NO: 102 - DNA, 1764 bp, synthetic construct
  misc_feature 1..1764: note = Synthetic
  source 1..1764: mol_type = other DNA
agtgggcatatcctggctggctgggatctcgtctgagaacacaatattactctggatgcaatagtcccctccagaagcctttggtccgtttccggaataaaacacgtacagaatgctattgttgcccaggaagcgtgagactggcctaggggtcagtctcaggctataagcgtagccggttccgggaattggagtggccacgggactagtagatgtcaggattcccccacttgggacgtgagattcgtagccggaacaggtgatattaaacttgttatcgccaggcagcacctggctaatttctccatcctccatgatgcattcaatgtcgatctcatttcccagcatctcggttttcacggagaagttgctatcctgggcgcttgtgggcagtgacagtggcagggtcacgtccagcccctgtgcccggaccactgcggtaatgttagtgctattacagttgtcccacttaatataagggacggtttcggggatcaggtacacaggattctgcatctcggcatggtggcaccacatggtgccaaacacatcctggaagtagacgtccactgattccagactgacctgctgttcctctcctgtggtcacattgatgggcagtttcttggacctcatggtcagagccagttctcctgctcccagcagctccagcagaaacagatttgttgcgttctcgctgcctccgaaagcccctctgggctgatacacggcttttgtatgtggggtcagctggccaaaatccaggtccagctggtgtttcttcccgcccacatcgaaattaatggtgacgttcacgtcggctgtacagacgttgcaggtggggtaaaatgggaactcagggatttccacattgaaaaatccagggtcctccccggtcagatgaatcaggctctgaatagtgtactggcacaccagcagggcggcttcgcctccgccagatccgcctccggacttcagcaggatcagggcctcggggttcaccacctggaaggtgaaggtctcggtgatgaacagcctcacggcgtccttctccctgtcctcgtagccgatgctcaggtcctggcccaggatcagcttgaagtcgccgcccctctcgctcaccaccagggcgtcctcgatcctgggggtggtgatgatcttgccgcccctcaggcactcctccaccctcttctccagggggtagtggccggcctcctccttcaggaagttgatccacctgtcggtgttgatcaccagggtgtaggggccctcgatgccgtccttgctgaagatgctcagggccctcacgatggcctccagcaggtccttgggggtgctgccgcactcgatcttcctctcctcgaagctcagcaggcccttcacgccgctcttctcgcagcccctgaagatcacctcgtcctcgaactcggccaccttcctcacggtctcctccaggctgctcaggtccacgttgggcttgcccctctccaggttgtccagctcccacaggtccagggtgaaggtggccctcagctcgatcaggggcaggctcttcctcaggccccacttcaccacctcgttctcgtcgctcagcacctccacctcgcccagggggtgggcggcgtactcccagccgtaggggccctccacgtccacgaacttcctgccgtacagctgggtcttgaagatctccctggccctgttgtcgatctcctgccactgcttctcggtcagaggggcgaagctcctcttcaggaactccat

SEQ ID NO: 103 - DNA, 2088 bp, synthetic construct
  misc_feature 1..2088: note = Synthetic
  source 1..2088: mol_type = other DNA
atggagttcctgaagaggagcttcgcccctctgaccgagaagcagtggcaggagatcgacaacagggccagggagatcttcaagacccagctgtacggcaggaagttcgtggacgtggagggcccctacggctgggagtacgccgcccaccccctgggcgaggtggaggtgctgagcgacgagaacgaggtggtgaagtggggcctgaggaagagcctgcccctgatcgagctgagggccaccttcaccctggacctgtgggagctggacaacctggagaggggcaagcccaacgtggacctgagcagcctggaggagaccgtgaggaaggtggccgagttcgaggacgaggtgatcttcaggggctgcgagaagagcggcgtgaagggcctgctgagcttcgaggagaggaagatcgagtgcggcagcacccccaaggacctgctggaggccatcgtgagggccctgagcatcttcagcaaggacggcatcgagggcccctacaccctggtgatcaacaccgacaggtggatcaacttcctgaaggaggaggccggccactaccccctggagaagagggtggaggagtgcctgaggggcggcaagatcatcaccacccccaggatcgaggacgccctggtggtgagcgagaggggcggcgacttcaagctgatcctgggccaggacctgagcatcggctacgaggacagggagaaggacgccgtgaggctgttcatcaccgagaccttcaccttccaggtggtgaaccccgaggccctgatcctgctgaagtccggaggcggatctggcggaggcgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctccc

SEQ ID NO: 104 - protein, 696 aa, synthetic construct
  REGION 1..696: note = Synthetic
MEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKSGGGSGGGEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAP

SEQ ID NO: 105 - DNA, 2088 bp, synthetic construct
  misc_feature 1..2088: note = Synthetic
  source 1..2088: mol_type = other DNA
gggagctttgctgaagatcactttgtgtgtggtagttgttgcgttagtagctgtccgggtaatgatcagggtttttggggcagtccccaggccggacactgtaatatcaaaggttcgattactagcgaaggcgccagagatgttttcacacccgctaggggtgcctgaagtcagtgtccatttgcacttaaaatcggtctcagtattgttgggccaggcccagaatgctgtgacggtcacatttggggagttagcgtcttcgcttgtgaccataggcacggaataagtggcattatcgcccacgtaggtaatgtcggtagtgttagtgggcatatcctggctggctgggatctcgtctgagaacacaatattactctggatgcaatagtcccctccagaagcctttggtccgtttccggaataaaacacgtacagaatgctattgttgcccaggaagcgtgagactggcctaggggtcagtctcaggctataagcgtagccggttccgggaattggagtggccacgggactagtagatgtcaggattcccccacttgggacgtgagattcgtagccggaacaggtgatattaaacttgttatcgccaggcagcacctggctaatttctccatcctccatgatgcattcaatgtcgatctcatttcccagcatctcggttttcacggagaagttgctatcctgggcgcttgtgggcagtgacagtggcagggtcacgtccagcccctgtgcccggaccactgcggtaatgttagtgctattacagttgtcccacttaatataagggacggtttcggggatcaggtacacaggattctgcatctcggcatggtggcaccacatggtgccaaacacatcctggaagtagacgtccactgattccagactgacctgctgttcctctcctgtggtcacattgatgggcagtttcttggacctcatggtcagagccagttctcctgctcccagcagctccagcagaaacagatttgttgcgttctcgctgcctccgaaagcccctctgggctgatacacggcttttgtatgtggggtcagctggccaaaatccaggtccagctggtgtttcttcccgcccacatcgaaattaatggtgacgttcacgtcggctgtacagacgttgcaggtggggtaaaatgggaactcagggatttccacattgaaaaatccagggtcctccccggtcagatgaatcaggctctgaatagtgtactggcacaccagcagggcggcttcgcctccgccagatccgcctccggacttcagcaggatcagggcctcggggttcaccacctggaaggtgaaggtctcggtgatgaacagcctcacggcgtccttctccctgtcctcgtagccgatgctcaggtcctggcccaggatcagcttgaagtcgccgcccctctcgctcaccaccagggcgtcctcgatcctgggggtggtgatgatcttgccgcccctcaggcactcctccaccctcttctccagggggtagtggccggcctcctccttcaggaagttgatccacctgtcggtgttgatcaccagggtgtaggggccctcgatgccgtccttgctgaagatgctcagggccctcacgatggcctccagcaggtccttgggggtgctgccgcactcgatcttcctctcctcgaagctcagcaggcccttcacgccgctcttctcgcagcccctgaagatcacctcgtcctcgaactcggccaccttcctcacggtctcctccaggctgctcaggtccacgttgggcttgcccctctccaggttgtccagctcccacaggtccagggtgaaggtggccctcagctcgatcaggggcaggctcttcctcaggccccacttcaccacctcgttctcgtcgctcagcacctccacctcgcccagggggtgggcggcgtactcccagccgtaggggccctccacgtccacgaacttcctgccgtacagctgggtcttgaagatctccctggccctgttgtcgatctcctgccactgcttctcggtcagaggggcgaagctcctcttcaggaactccat

SEQ ID NO: 106 - DNA, 6005 bp, synthetic construct
  misc_feature 1..6005: note = Synthetic
  source 1..6005: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggacagcaagggcagcagccagaagggcagcagactgctgctgctgctggtggtgagcaacctgctgctgcctcagggcgtgctagccgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccacttccggagagagccaggtgaggcagaacttcaagcccgagatggaggagaagctgaacgagcagatgaacctggagctgtacagcagcctgctgtaccagcagatgagcgcctggtgcagctaccacaccttcgagggcgccgccgccttcctgaggaggcacgcccaggaggagatgacccacatgcagaggctgttcgactacctgaccgacaccggcaacctgcccaggatcaacaccgtggagagccccttcgccgagtacagcagcctggacgagctgttccaggagacctacaagcacgagcagctgatcacccagaagatcaacgagctggcccacgccgccatgaccaaccaggactaccccaccttcaacttcctgcagtggtacgtgagcgagcagcacgaggaggagaagctgttcaagagcatcatcgacaagctgagcctggccggcaagagcggcgagggcctgtacttcatcgacaaggagctgagcaccctggacggatcctagcatcatcatcatcattagtctggaagggcgaattgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 107 - DNA, 6329 bp, synthetic construct
  misc_feature 1..6329: note = Synthetic
  source 1..6329: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggacagcaagggcagcagccagaagggcagcagactgctgctgctgctggtggtgagcaacctgctgctgcctcagggcgtgctagccgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctccctccggagagagccaggtgaggcagaacttcaagcccgagatggaggagaagctgaacgagcagatgaacctggagctgtacagcagcctgctgtaccagcagatgagcgcctggtgcagctaccacaccttcgagggcgccgccgccttcctgaggaggcacgcccaggaggagatgacccacatgcagaggctgttcgactacctgaccgacaccggcaacctgcccaggatcaacaccgtggagagccccttcgccgagtacagcagcctggacgagctgttccaggagacctacaagcacgagcagctgatcacccagaagatcaacgagctggcccacgccgccatgaccaaccaggactaccccaccttcaacttcctgcagtggtacgtgagcgagcagcacgaggaggagaagctgttcaagagcatcatcgacaagctgagcctggccggcaagagcggcgagggcctgtacttcatcgacaaggagctgagcaccctggacggatcctagcatcatcatcatcattagtctggaagggcgaattgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 108 - DNA, 6464 bp, synthetic construct
  misc_feature 1..6464: note = Synthetic
  source 1..6464: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggacagcaagggcagcagccagaagggcagcagactgctgctgctgctggtggtgagcaacctgctgctgcctcagggcgtgctagccgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgagtccactaccacatctcctaccctgaacactaccgggtttgccgaccccaatacaactaccggactgcctagctccacccatgtgccaacaaacctgactgcaccagcatccaccggacctacagtgtctacttccggagagagccaggtgaggcagaacttcaagcccgagatggaggagaagctgaacgagcagatgaacctggagctgtacagcagcctgctgtaccagcagatgagcgcctggtgcagctaccacaccttcgagggcgccgccgccttcctgaggaggcacgcccaggaggagatgacccacatgcagaggctgttcgactacctgaccgacaccggcaacctgcccaggatcaacaccgtggagagccccttcgccgagtacagcagcctggacgagctgttccaggagacctacaagcacgagcagctgatcacccagaagatcaacgagctggcccacgccgccatgaccaaccaggactaccccaccttcaacttcctgcagtggtacgtgagcgagcagcacgaggaggagaagctgttcaagagcatcatcgacaagctgagcctggccggcaagagcggcgagggcctgtacttcatcgacaaggagctgagcaccctggacggatcctagcatcatcatcatcattagtctggaagggcgaattgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 109 - DNA, 7634 bp, synthetic construct
  misc_feature 1..7634: note = Synthetic
  source 1..7634: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggacagcaagggcagcagccagaagggcagcagactgctgctgctgctggtggtgagcaacctgctgctgcctcagggcgtgctagccgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgagtccactaccacatctcctaccctgaacactaccgggtttgccgaccccaatacaactaccggactgcctagctccacccatgtgccaacaaacctgactgcaccagcatccaccggacctacagtgtctactgccgatgtcaccagtcccacacctgccggaacaacttctggcgctagtcccgtgaccccatcacccagcccttgggacaatgggacagagagtaaggcccctgatatgacttctagtacctcaccagtcaccacaccaacccccaacgcaacaagccctactccagccgtgactacccccacacctaatgctaccagcccaacacccgcagtgacaactcctaccccaaacgccacttccccaaccctggggaagacatcacccactagcgccgtgaccacacccacccctaatgctacctctcctacactgggaaaaacttccccaacctctgcagtgactaccccaacccccaacgccacaagccccactctgggcaagaccagtcctacatcagctgtcacaactcctaccccaaatgcaactgggccaaccgtgggagagacatccccccaggctaacgcaacaaatcacactctgggaggcaccagtcccacacctgtggtcacctcacagcccaagaacgccacaagcgctgtgaccacaggccagcataatatcacatcaagctccacttctagtatgagcctgcgcccttcaagcaacccagagacactgtccccatctactagtgacaattcaaccagccacatgcctctgctgacatctgcacatccaactgggggagaaaacatcactcaggtcacccccgcctccatttctacccaccatgtgtccacatcctctccagcaccccgacctggaactaccagccaggcatccggaccaggaaatagttcaaccagcacaaagcctggcgaggtgaacgtcacaaaagggactccccctcagaatgctacctcacctcaggcaccaagcggccagaaaacagctgtgcctactgtcacctccacaggcgggaaggcaaactctacaactggaggcaaacacaccacagggcatggagctcgcactagcaccgaaccaactaccgactacgggggagattccacaactccaaggcccagatacaatgccaccacatatctgccaccctctaccagctccaagctgcgacccagatggacattcactagtcctccagtgactaccgcacaggctacagtgccagtcccacctacttctcagcctagattttctaacctgagttccggagagagccaggtgaggcagaacttcaagcccgagatggaggagaagctgaacgagcagatgaacctggagctgtacagcagcctgctgtaccagcagatgagcgcctggtgcagctaccacaccttcgagggcgccgccgccttcctgaggaggcacgcccaggaggagatgacccacatgcagaggctgttcgactacctgaccgacaccggcaacctgcccaggatcaacaccgtggagagccccttcgccgagtacagcagcctggacgagctgttccaggagacctacaagcacgagcagctgatcacccagaagatcaacgagctggcccacgccgccatgaccaaccaggactaccccaccttcaacttcctgcagtggtacgtgagcgagcagcacgaggaggagaagctgttcaagagcatcatcgacaagctgagcctggccggcaagagcggcgagggcctgtacttcatcgacaaggagctgagcaccctggacggatcctagcatcatcatcatcattagtctggaagggcgaattgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 110 - DNA, 6020 bp, synthetic construct
  misc_feature 1..6020: note = Synthetic
  source 1..6020: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggacagcaagggcagcagccagaagggcagcagactgctgctgctgctggtggtgagcaacctgctgctgcctcagggcgtgctagccgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccacttccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctagcatcatcatcatcattagtctggaagggcgaattgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 111 - DNA, 6344 bp, synthetic construct
  misc_feature 1..6344: note = Synthetic
  source 1..6344: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggacagcaagggcagcagccagaagggcagcagactgctgctgctgctggtggtgagcaacctgctgctgcctcagggcgtgctagccgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctccctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctagcatcatcatcatcattagtctggaagggcgaattgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 112 - DNA, 6479 bp, synthetic construct
  misc_feature 1..6479: note = Synthetic
  source 1..6479: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggacagcaagggcagcagccagaagggcagcagactgctgctgctgctggtggtgagcaacctgctgctgcctcagggcgtgctagccgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgagtccactaccacatctcctaccctgaacactaccgggtttgccgaccccaatacaactaccggactgcctagctccacccatgtgccaacaaacctgactgcaccagcatccaccggacctacagtgtctacttccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctagcatcatcatcatcattagtctggaagggcgaattgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 113 - DNA, 7649 bp, synthetic construct
  misc_feature 1..7649: note = Synthetic
  source 1..7649: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggacagcaagggcagcagccagaagggcagcagactgctgctgctgctggtggtgagcaacctgctgctgcctcagggcgtgctagccgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgagtccactaccacatctcctaccctgaacactaccgggtttgccgaccccaatacaactaccggactgcctagctccacccatgtgccaacaaacctgactgcaccagcatccaccggacctacagtgtctactgccgatgtcaccagtcccacacctgccggaacaacttctggcgctagtcccgtgaccccatcacccagcccttgggacaatgggacagagagtaaggcccctgatatgacttctagtacctcaccagtcaccacaccaacccccaacgcaacaagccctactccagccgtgactacccccacacctaatgctaccagcccaacacccgcagtgacaactcctaccccaaacgccacttccccaaccctggggaagacatcacccactagcgccgtgaccacacccacccctaatgctacctctcctacactgggaaaaacttccccaacctctgcagtgactaccccaacccccaacgccacaagccccactctgggcaagaccagtcctacatcagctgtcacaactcctaccccaaatgcaactgggccaaccgtgggagagacatccccccaggctaacgcaacaaatcacactctgggaggcaccagtcccacacctgtggtcacctcacagcccaagaacgccacaagcgctgtgaccacaggccagcataatatcacatcaagctccacttctagtatgagcctgcgcccttcaagcaacccagagacactgtccccatctactagtgacaattcaaccagccacatgcctctgctgacatctgcacatccaactgggggagaaaacatcactcaggtcacccccgcctccatttctacccaccatgtgtccacatcctctccagcaccccgacctggaactaccagccaggcatccggaccaggaaatagttcaaccagcacaaagcctggcgaggtgaacgtcacaaaagggactccccctcagaatgctacctcacctcaggcaccaagcggccagaaaacagctgtgcctactgtcacctccacaggcgggaaggcaaactctacaactggaggcaaacacaccacagggcatggagctcgcactagcaccgaaccaactaccgactacgggggagattccacaactccaaggcccagatacaatgccaccacatatctgccaccctctaccagctccaagctgcgacccagatggacattcactagtcctccagtgactaccgcacaggctacagtgccagtcccacctacttctcagcctagattttctaacctgagttccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctagcatcatcatcatcattagtctggaagggcgaattgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 114 - DNA, 6291 bp, synthetic construct
  misc_feature 1..6291: note = Synthetic
  source 1..6291: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagacaccatgcccatgggcagcctgcagcccctggccaccctgtacctgctgggcatgctggtggctagcgtgctggccatggagttcctgaagaggagcttcgcccctctgaccgagaagcagtggcaggagatcgacaacagggccagggagatcttcaagacccagctgtacggcaggaagttcgtggacgtggagggcccctacggctgggagtacgccgcccaccccctgggcgaggtggaggtgctgagcgacgagaacgaggtggtgaagtggggcctgaggaagagcctgcccctgatcgagctgagggccaccttcaccctggacctgtgggagctggacaacctggagaggggcaagcccaacgtggacctgagcagcctggaggagaccgtgaggaaggtggccgagttcgaggacgaggtgatcttcaggggctgcgagaagagcggcgtgaagggcctgctgagcttcgaggagaggaagatcgagtgcggcagcacccccaaggacctgctggaggccatcgtgagggccctgagcatcttcagcaaggacggcatcgagggcccctacaccctggtgatcaacaccgacaggtggatcaacttcctgaaggaggaggccggccactaccccctggagaagagggtggaggagtgcctgaggggcggcaagatcatcaccacccccaggatcgaggacgccctggtggtgagcgagaggggcggcgacttcaagctgatcctgggccaggacctgagcatcggctacgaggacagggagaaggacgccgtgaggctgttcatcaccgagaccttcaccttccaggtggtgaaccccgaggccctgatcctgctgaagtccggaggcggatctggcggaggcgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccacttgatgaggatcccatcatcatcatcatcattagtctggaagggcgaattgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 115 - DNA, 6615 bp, synthetic construct
  misc_feature 1..6615: note = Synthetic
  source 1..6615: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagacaccatgcccatgggcagcctgcagcccctggccaccctgtacctgctgggcatgctggtggctagcgtgctggccatggagttcctgaagaggagcttcgcccctctgaccgagaagcagtggcaggagatcgacaacagggccagggagatcttcaagacccagctgtacggcaggaagttcgtggacgtggagggcccctacggctgggagtacgccgcccaccccctgggcgaggtggaggtgctgagcgacgagaacgaggtggtgaagtggggcctgaggaagagcctgcccctgatcgagctgagggccaccttcaccctggacctgtgggagctggacaacctggagaggggcaagcccaacgtggacctgagcagcctggaggagaccgtgaggaaggtggccgagttcgaggacgaggtgatcttcaggggctgcgagaagagcggcgtgaagggcctgctgagcttcgaggagaggaagatcgagtgcggcagcacccccaaggacctgctggaggccatcgtgagggccctgagcatcttcagcaaggacggcatcgagggcccctacaccctggtgatcaacaccgacaggtggatcaacttcctgaaggaggaggccggccactaccccctggagaagagggtggaggagtgcctgaggggcggcaagatcatcaccacccccaggatcgaggacgccctggtggtgagcgagaggggcggcgacttcaagctgatcctgggccaggacctgagcatcggctacgaggacagggagaaggacgccgtgaggctgttcatcaccgagaccttcaccttccaggtggtgaaccccgaggccctgatcctgctgaagtccggaggcggatctggcggaggcgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctccctgatgaggatcccatcatcatcatcatcattagtctggaagggcgaattgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 116 - DNA, 6750 bp, synthetic construct
  misc_feature 1..6750: note = Synthetic
  source 1..6750: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagacaccatgcccatgggcagcctgcagcccctggccaccctgtacctgctgggcatgctggtggctagcgtgctggccatggagttcctgaagaggagcttcgcccctctgaccgagaagcagtggcaggagatcgacaacagggccagggagatcttcaagacccagctgtacggcaggaagttcgtggacgtggagggcccctacggctgggagtacgccgcccaccccctgggcgaggtggaggtgctgagcgacgagaacgaggtggtgaagtggggcctgaggaagagcctgcccctgatcgagctgagggccaccttcaccctggacctgtgggagctggacaacctggagaggggcaagcccaacgtggacctgagcagcctggaggagaccgtgaggaaggtggccgagttcgaggacgaggtgatcttcaggggctgcgagaagagcggcgtgaagggcctgctgagcttcgaggagaggaagatcgagtgcggcagcacccccaaggacctgctggaggccatcgtgagggccctgagcatcttcagcaaggacggcatcgagggcccctacaccctggtgatcaacaccgacaggtggatcaacttcctgaaggaggaggccggccactaccccctggagaagagggtggaggagtgcctgaggggcggcaagatcatcaccacccccaggatcgaggacgccctggtggtgagcgagaggggcggcgacttcaagctgatcctgggccaggacctgagcatcggctacgaggacagggagaaggacgccgtgaggctgttcatcaccgagaccttcaccttccaggtggtgaaccccgaggccctgatcctgctgaagtccggaggcggatctggcggaggcgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgagtccactaccacatctcctaccctgaacactaccgggtttgccgaccccaatacaactaccggactgcctagctccacccatgtgccaacaaacctgactgcaccagcatccaccggacctacagtgtctacttgatgaggatcccatcatcatcatcatcattagtctggaagggcgaattgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 117 - DNA, 7920 bp, synthetic construct
  misc_feature 1..7920: note = Synthetic
  source 1..7920: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagacaccatgcccatgggcagcctgcagcccctggccaccctgtacctgctgggcatgctggtggctagcgtgctggccatggagttcctgaagaggagcttcgcccctctgaccgagaagcagtggcaggagatcgacaacagggccagggagatcttcaagacccagctgtacggcaggaagttcgtggacgtggagggcccctacggctgggagtacgccgcccaccccctgggcgaggtggaggtgctgagcgacgagaacgaggtggtgaagtggggcctgaggaagagcctgcccctgatcgagctgagggccaccttcaccctggacctgtgggagctggacaacctggagaggggcaagcccaacgtggacctgagcagcctggaggagaccgtgaggaaggtggccgagttcgaggacgaggtgatcttcaggggctgcgagaagagcggcgtgaagggcctgctgagcttcgaggagaggaagatcgagtgcggcagcacccccaaggacctgctggaggccatcgtgagggccctgagcatcttcagcaaggacggcatcgagggcccctacaccctggtgatcaacaccgacaggtggatcaacttcctgaaggaggaggccggccactaccccctggagaagagggtggaggagtgcctgaggggcggcaagatcatcaccacccccaggatcgaggacgccctggtggtgagcgagaggggcggcgacttcaagctgatcctgggccaggacctgagcatcggctacgaggacagggagaaggacgccgtgaggctgttcatcaccgagaccttcaccttccaggtggtgaaccccgaggccctgatcctgctgaagtccggaggcggatctggcggaggcgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgagtccactaccacatctcctaccctgaacactaccgggtttgccgaccccaatacaactaccggactgcctagctccacccatgtgccaacaaacctgactgcaccagcatccaccggacctacagtgtctactgccgatgtcaccagtcccacacctgccggaacaacttctggcgctagtcccgtgaccccatcacccagcccttgggacaatgggacagagagtaaggcccctgatatgacttctagtacctcaccagtcaccacaccaacccccaacgcaacaagccctactccagccgtgactacccccacacctaatgctaccagcccaacacccgcagtgacaactcctaccccaaacgccacttccccaaccctggggaagacatcacccactagcgccgtgaccacacccacccctaatgctacctctcctacactgggaaaaacttccccaacctctgcagtgactaccccaacccccaacgccacaagccccactctgggcaagaccagtcctacatcagctgtcacaactcctaccccaaatgcaactgggccaaccgtgggagagacatccccccaggctaacgcaacaaatcacactctgggaggcaccagtcccacacctgtggtcacctcacagcccaagaacgccacaagcgctgtgaccacaggccagcataatatcacatcaagctccacttctagtatgagcctgcgcccttcaagcaacccagagacactgtccccatctactagtgacaattcaaccagccacatgcctctgctgacatctgcacatccaactgggggagaaaacatcactcaggtcacccccgcctccatttctacccaccatgtgtccacatcctctccagcaccccgacctggaactaccagccaggcatccggaccaggaaatagttcaaccagcacaaagcctggcgaggtgaacgtcacaaaagggactccccctcagaatgctacctcacctcaggcaccaagcggccagaaaacagctgtgcctactgtcacctccacaggcgggaaggcaaactctacaactggaggcaaacacaccacagggcatggagctcgcactagcaccgaaccaactaccgactacgggggagattccacaactccaaggcccagatacaatgccaccacatatctgccaccctctaccagctccaagctgcgacccagatggacattcactagtcctccagtgactaccgcacaggctacagtgccagtcccacctacttctcagcctagattttctaacctgagttgatgaggatcccatcatcatcatcatcattagtctggaagggcgaattgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 118 - DNA, 5031 bp, synthetic construct
  misc_feature 1..5031: note = Synthetic
  source 1..5031: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagacaccatgcccatgggcagcctgcagcccctggccaccctgtacctgctgggcatgctggtggctagcgtgctggcctccggagagagccaggtgaggcagaacttcaagcccgagatggaggagaagctgaacgagcagatgaacctggagctgtacagcagcctgctgtaccagcagatgagcgcctggtgcagctaccacaccttcgagggcgccgccgccttcctgaggaggcacgcccaggaggagatgacccacatgcagaggctgttcgactacctgaccgacaccggcaacctgcccaggatcaacaccgtggagagccccttcgccgagtacagcagcctggacgagctgttccaggagacctacaagcacgagcagctgatcacccagaagatcaacgagctggcccacgccgccatgaccaaccaggactaccccaccttcaacttcctgcagtggtacgtgagcgagcagcacgaggaggagaagctgttcaagagcatcatcgacaagctgagcctggccggcaagagcggcgagggcctgtacttcatcgacaaggagctgagcaccctggacggatcctagcatcatcatcatcattagtctggaagggcgaattgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 119 - DNA, 5046 bp, synthetic construct
  misc_feature 1..5046: note = Synthetic
  source 1..5046: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagacaccatgcccatgggcagcctgcagcccctggccaccctgtacctgctgggcatgctggtggctagcgtgctggcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctagcatcatcatcatcattagtctggaagggcgaattgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 120 - DNA, 5322 bp, synthetic construct
  misc_feature 1..5322: note = Synthetic
  source 1..5322: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagacaccatgcccatgggcagcctgcagcccctggccaccctgtacctgctgggcatgctggtggctagcgtgctggccatggagttcctgaagaggagcttcgcccctctgaccgagaagcagtggcaggagatcgacaacagggccagggagatcttcaagacccagctgtacggcaggaagttcgtggacgtggagggcccctacggctgggagtacgccgcccaccccctgggcgaggtggaggtgctgagcgacgagaacgaggtggtgaagtggggcctgaggaagagcctgcccctgatcgagctgagggccaccttcaccctggacctgtgggagctggacaacctggagaggggcaagcccaacgtggacctgagcagcctggaggagaccgtgaggaaggtggccgagttcgaggacgaggtgatcttcaggggctgcgagaagagcggcgtgaagggcctgctgagcttcgaggagaggaagatcgagtgcggcagcacccccaaggacctgctggaggccatcgtgagggccctgagcatcttcagcaaggacggcatcgagggcccctacaccctggtgatcaacaccgacaggtggatcaacttcctgaaggaggaggccggccactaccccctggagaagagggtggaggagtgcctgaggggcggcaagatcatcaccacccccaggatcgaggacgccctggtggtgagcgagaggggcggcgacttcaagctgatcctgggccaggacctgagcatcggctacgaggacagggagaaggacgccgtgaggctgttcatcaccgagaccttcaccttccaggtggtgaaccccgaggccctgatcctgctgaagtccggaggcggatcccatcatcatcatcatcattagtctggaagggcgaattgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 121 - DNA, 7135 bp, synthetic construct
  misc_feature 1..7135: note = Synthetic
  source 1..7135: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagacaccatggaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgagtccactaccacatctcctaccctgaacactaccgggtttgccgaccccaatacaactaccggactgcctagctccacccatgtgccaacaaacctgactgcaccagcatccaccggacctacagtgtctactgccgatgtcaccagtcccacacctgccggaacaacttctggcgctagtcccgtgaccccatcacccagcccttgggacaatgggacagagagtaaggcccctgatatgacttctagtacctcaccagtcaccacaccaacccccaacgcaacaagccctactccagccgtgactacccccacacctaatgctaccagcccaacacccgcagtgacaactcctaccccaaacgccacttccccaaccctggggaagacatcacccactagcgccgtgaccacacccacccctaatgctacctctcctacactgggaaaaacttccccaacctctgcagtgactaccccaacccccaacgccacaagccccactctgggcaagaccagtcctacatcagctgtcacaactcctaccccaaatgcaactgggccaaccgtgggagagacatccccccaggctaacgcaacaaatcacactctgggaggcaccagtcccacacctgtggtcacctcacagcccaagaacgccacaagcgctgtgaccacaggccagcataatatcacatcaagctccacttctagtatgagcctgcgcccttcaagcaacccagagacactgtccccatctactagtgacaattcaaccagccacatgcctctgctgacatctgcacatccaactgggggagaaaacatcactcaggtcacccccgcctccatttctacccaccatgtgtccacatcctctccagcaccccgacctggaactaccagccaggcatccggaccaggaaatagttcaaccagcacaaagcctggcgaggtgaacgtcacaaaagggactccccctcagaatgctacctcacctcaggcaccaagcggccagaaaacagctgtgcctactgtcacctccacaggcgggaaggcaaactctacaactggaggcaaacacaccacagggcatggagctcgcactagcaccgaaccaactaccgactacgggggagattccacaactccaaggcccagatacaatgccaccacatatctgccaccctctaccagctccaagctgcgacccagatggacattcactagtcctccagtgactaccgcacaggctacagtgccagtcccacctacttctcagcctagattttctaacctgagtatgctggtgctgcagtgggcaagcctggcagtcctgaccctgctgctgctgctggtcatggctgactgtgcattccggagaaacctgtccacttcacacacttacaccacccccccttacgatgacgcagagacttatgtctgataggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 122 - DNA, 7148 bp, synthetic construct
  misc_feature 1..7148: note = Synthetic
  source 1..7148: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggacagcaagggcagcagccagaagggcagcagactgctgctgctgctggtggtgagcaacctgctgctgcctcagggcgtgctagccgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctcccgagtccactaccacatctcctaccctgaacactaccgggtttgccgaccccaatacaactaccggactgcctagctccacccatgtgccaacaaacctgactgcaccagcatccaccggacctacagtgtctactgccgatgtcaccagtcccacacctgccggaacaacttctggcgctagtcccgtgaccccatcacccagcccttgggacaatgggacagagagtaaggcccctgatatgacttctagtacctcaccagtcaccacaccaacccccaacgcaacaagccctactccagccgtgactacccccacacctaatgctaccagcccaacacccgcagtgacaactcctaccccaaacgccacttccccaaccctggggaagacatcacccactagcgccgtgaccacacccacccctaatgctacctctcctacactgggaaaaacttccccaacctctgcagtgactaccccaacccccaacgccacaagccccactctgggcaagaccagtcctacatcagctgtcacaactcctaccccaaatgcaactgggccaaccgtgggagagacatccccccaggctaacgcaacaaatcacactctgggaggcaccagtcccacacctgtggtcacctcacagcccaagaacgccacaagcgctgtgaccacaggccagcataatatcacatcaagctccacttctagtatgagcctgcgcccttcaagcaacccagagacactgtccccatctactagtgacaattcaaccagccacatgcctctgctgacatctgcacatccaactgggggagaaaacatcactcaggtcacccccgcctccatttctacccaccatgtgtccacatcctctccagcaccccgacctggaactaccagccaggcatccggaccaggaaatagttcaaccagcacaaagcctggcgaggtgaacgtcacaaaagggactccccctcagaatgctacctcacctcaggcaccaagcggccagaaaacagctgtgcctactgtcacctccacaggcgggaaggcaaactctacaactggaggcaaacacaccacagggcatggagctcgcactagcaccgaaccaactaccgactacgggggagattccacaactccaaggcccagatacaatgccaccacatatctgccaccctctaccagctccaagctgcgacccagatggacattcactagtcctccagtgactaccgcacaggctacagtgccagtcccacctacttctcagcctagattttctaacctgagtcaccaccaccaccaccactgatgaggatcctagcatcatcatcatcattagtctggaagggcgaattgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 123 - DNA, 5843 bp, synthetic construct
  misc_feature 1..5843: note = Synthetic
  source 1..5843: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggacagcaagggcagcagccagaagggcagcagactgctgctgctgctggtggtgagcaacctgctgctgcctcagggcgtgctagccgaagccgccctgctggtgtgccagtacactattcagagcctgattcatctgaccggggaggaccctggatttttcaatgtggaaatccctgagttcccattttaccccacctgcaacgtctgtacagccgacgtgaacgtcaccattaatttcgatgtgggcgggaagaaacaccagctggacctggattttggccagctgaccccacatacaaaagccgtgtatcagcccagaggggctttcggaggcagcgagaacgcaacaaatctgtttctgctggagctgctgggagcaggagaactggctctgaccatgaggtccaagaaactgcccatcaatgtgaccacaggagaggaacagcaggtcagtctggaatcagtggacgtctacttccaggatgtgtttggcaccatgtggtgccaccatgccgagatgcagaatcctgtgtacctgatccccgaaaccgtcccttatattaagtgggacaactgtaatagcactaacattaccgcagtggtccgggcacaggggctggacgtgaccctgccactgtcactgcccacaagcgcccaggatagcaacttctccgtgaaaaccgagatgctgggaaatgagatcgacattgaatgcatcatggaggatggagaaattagccaggtgctgcctggcgataacaagtttaatatcacctgttccggctacgaatctcacgtcccaagtgggggaatcctgacatctactagtcccgtggccactccaattcccggaaccggctacgcttatagcctgagactgacccctaggccagtctcacgcttcctgggcaacaatagcattctgtacgtgttttattccggaaacggaccaaaggcttctggaggggactattgcatccagagtaatattgtgttctcagacgagatcccagccagccaggatatgcccactaacactaccgacattacctacgtgggcgataatgccacttattccgtgcctatggtcacaagcgaagacgctaactccccaaatgtgaccgtcacagcattctgggcctggcccaacaatactgagaccgattttaagtgcaaatggacactgacttcaggcacccctagcgggtgtgaaaacatctctggcgccttcgctagtaatcgaacctttgatattacagtgtccggcctggggactgccccaaaaaccctgatcattacccggacagctactaacgcaacaactaccacacacaaagtgatcttcagcaaagctccccaccaccaccaccaccactgatgaggatcctagcatcatcatcatcattagtctggaagggcgaattgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 124 - DNA, 6533 bp, synthetic construct
  misc_feature 1..6533: note = Synthetic
  source 1..6533: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgcttctagacaccatgcagctcctgtgcgtgttctgtctggtgctgctgtgggaagtgggagccgcttctctgagtgaggtgaagctgcacctggacattgaaggccacgcctcccattacactatcccttggaccgagctgatggctaaagtgccaggactgtctcctgaggctctgtggcgggaagctaatgtgaccgaggatctggcctctatgctgaacagatacaagctgatctataaaaccagtggcacactggggattgctctggctgagccagtggacatccccgccgtgtcagaaggaagcatgcaggtggatgctagtaaggtgcatccaggggtgattagcggactgaacagcccagcttgcatgctgagcgctcctctggagaaacagctcttctactatatcggcaccatgctgcctaatacacggccacacagctacgtgttttatcagctcagatgtcatctgtcctacgtggccctgtctattaacggggacaagttccagtatacaggagctatgacttccaaatttctgatgggaacttacaagcgggtgaccgagaaaggcgatgaacacgtgctgtctctggtgttcgggaagacaaaagacctgcccgatctgagaggacccttttcctacccttctctgactagtgcccagtcaggcgactatagcctggtgatcgtgaccacattcgtgcactacgctaacttccataattattttgtgcccaatctgaaggatatgttttcccgggccgtgaccatgacagccgcttcttacgctagatatgtgctgcagaagctggtgctgctggagatgaaaggcgggtgccgggagcctgaactggacactgaaaccctgactaccatgttcgaggtgtccgtggccttctttaaagtgggacacgctgtgggagagacaggaaacggatgcgtggacctgagatggctggccaagagcttctttgaactgaccgtgctgaaagatatcattggaatctgttacggcgccacagtgaaaggaatgcagagctatggcctggagaggctggccgctatgctgatggccaccgtgaagatggaggaactgggccacctgacaactgagaaacaggaatacgctctgaggctggctaccgtgggatacccaaaggccggggtgtattccggactgattggaggcgccacatctgtgctgctgagtgcttataataggcacccactgttccagcccctgcatacagtgatgcgcgagactctgtttatcgggtctcatgtggtgctgcgggaactgagactgaatgtgaccacacagggacccaacctggccctgtaccagctcctgagtactgccctgtgctcagctctggagattggagaagtgctgaggggactggccctggggaccgagtcaggactgttcagcccttgttatctgtcactgaggtttgacctgactcgcgataagctgctgagcatggccccacaggaagctaccctggaccaggccgctgtgagcaatgccgtggatggattcctgggcaggctgtccctggagagggaagaccgcgatgcctggcacctgccagcttacaagtgcgtggaccgcctggataaagtgctgatgatcattcccctgatcaacgtgaccttcatcattagctccgacagggaagtgagaggcagcgctctgtacgaagcttccactacctatctgtctagttcactgtttctgtcacctgtgattatgaataagtgtagccagggagctgtggctggagagcccagacagatcccaaagattcagaacttcacacgcactcagaaaagttgcatcttctgtggctttgccctgctgtcatacgatgagaaagaagggctggagacaactacctatattacatctcaggaagtgcagaacagtatcctgagctccaattacttcgactttgataacctgcacgtgcattatctgctgctgacaactaacggcaccgtgatggagatcgctggactgtacgaggaaagggctcacgtggtgctggctatcattctgtatttcatcgcctttgctctgggcatttttctggtgcataagatcgtgatgttctttctgtgataggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 125 - DNA, 4826 bp, synthetic construct
  misc_feature 1..4826: note = Synthetic
  source 1..4826: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgcttctagacaccatgcgggccgtgggggtgttcctggctatctgcctggtgactatttttgtgctgccaacctggggaaactgggcttacccttgctgtcacgtgacccagctcagggcccagcatctgctggctctggagaacatcagcgacatttatctggtgtccaatcagacatgcgatgggttcagcctggcctccctgaacagccccaagaacggatctaatcagctcgtgatctcccggtgtgctaacggcctgaatgtcgtgagtttctttatctcaattctgaaaaggagctcctctgctctgacaggacacctgagggagctgctgaccacactggaaactctgtacggaagtttctcagtggaagacctgtttggcgccaacctgaatcggtatgcttggcatagaggcgggtgataggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 126 - DNA, 5084 bp, synthetic construct
  misc_feature 1..5084: note = Synthetic
  source 1..5084: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgcttctagacaccatggtcagcttcaaacaagtgcgggtgcccctgtttactgccatcgctctggtgattgtgctgctgctggcctacttcctgccacctcgggtcagaggaggaggaagagtggccgctgccgctatcacctgggtgccaaaacctaatgtggaagtgtggcctgtggacccaccacctccagtgaactttaataagacagccgagcaggaatatggcgataaagaagtgaagctgcctcactggaccccaacactgcatacattccaggtgccacagaactacactaaagctaattgcacttattgtaacaccagggagtacacatttagttataaggggtgctgtttctactttactaagaaaaagcacacctggaatggatgcttccaggcctgtgctgaactgtatccatgcacatacttttatggcccaactcccgacatcctgcccgtggtgaccaggaacctgaatgccattgagtccctgtgggtgggagtgtacagggtgggagaaggcaactggacctccctggatggcgggacattcaaagtgtaccagatttttggctctcattgcacttatgtgtctaagttcagtaccgtgcccgtgtcacaccatgagtgtagctttctgaagccttgcctgtgtgtgtctcagagaagcaactcctgataggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 127 - DNA, 2580 bp, synthetic construct
  misc_feature 1..2580: note = Synthetic
  source 1..2580: mol_type = other DNA
atgcagctcctgtgcgtgttctgtctggtgctgctgtgggaagtgggagccgcttctctgagtgaggtgaagctgcacctggacattgaaggccacgcctcccattacactatcccttggaccgagctgatggctaaagtgccaggactgtctcctgaggctctgtggcgggaagctaatgtgaccgaggatctggcctctatgctgaacagatacaagctgatctataaaaccagtggcacactggggattgctctggctgagccagtggacatccccgccgtgtcagaaggaagcatgcaggtggatgctagtaaggtgcatccaggggtgattagcggactgaacagcccagcttgcatgctgagcgctcctctggagaaacagctcttctactatatcggcaccatgctgcctaatacacggccacacagctacgtgttttatcagctcagatgtcatctgtcctacgtggccctgtctattaacggggacaagttccagtatacaggagctatgacttccaaatttctgatgggaacttacaagcgggtgaccgagaaaggcgatgaacacgtgctgtctctggtgttcgggaagacaaaagacctgcccgatctgagaggacccttttcctacccttctctgactagtgcccagtcaggcgactatagcctggtgatcgtgaccacattcgtgcactacgctaacttccataattattttgtgcccaatctgaaggatatgttttcccgggccgtgaccatgacagccgcttcttacgctagatatgtgctgcagaagctggtgctgctggagatgaaaggcgggtgccgggagcctgaactggacactgaaaccctgactaccatgttcgaggtgtccgtggccttctttaaagtgggacacgctgtgggagagacaggaaacggatgcgtggacctgagatggctggccaagagcttctttgaactgaccgtgctgaaagatatcattggaatctgttacggcgccacagtgaaaggaatgcagagctatggcctggagaggctggccgctatgctgatggccaccgtgaagatggaggaactgggccacctgacaactgagaaacaggaatacgctctgaggctggctaccgtgggatacccaaaggccggggtgtattccggactgattggaggcgccacatctgtgctgctgagtgcttataataggcacccactgttccagcccctgcatacagtgatgcgcgagactctgtttatcgggtctcatgtggtgctgcgggaactgagactgaatgtgaccacacagggacccaacctggccctgtaccagctcctgagtactgccctgtgctcagctctggagattggagaagtgctgaggggactggccctggggaccgagtcaggactgttcagcccttgttatctgtcactgaggtttgacctgactcgcgataagctgctgagcatggccccacaggaagctaccctggaccaggccgctgtgagcaatgccgtggatggattcctgggcaggctgtccctggagagggaagaccgcgatgcctggcacctgccagcttacaagtgcgtggaccgcctggataaagtgctgatgatcattcccctgatcaacgtgaccttcatcattagctccgacagggaagtgagaggcagcgctctgtacgaagcttccactacctatctgtctagttcactgtttctgtcacctgtgattatgaataagtgtagccagggagctgtggctggagagcccagacagatcccaaagattcagaacttcacacgcactcagaaaagttgcatcttctgtggctttgccctgctgtcatacgatgagaaagaagggctggagacaactacctatattacatctcaggaagtgcagaacagtatcctgagctccaattacttcgactttgataacctgcacgtgcattatctgctgctgacaactaacggcaccgtgatggagatcgctggactgtacgaggaaagggctcactctggcggctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 128 - protein, 859 aa, synthetic construct
  REGION 1..859: note = Synthetic
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHSGGSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 129 - DNA, 645 bp, synthetic construct
  misc_feature 1..645: note = Synthetic
  source 1..645: mol_type = other DNA
atgcccatgggcagcctgcagcccctggccaccctgtacctgctgggcatgctggtggctagcgtgctggccggaggaagagtggccgctgccgctatcacctgggtgccaaaacctaatgtggaagtgtggcctgtggacccaccacctccagtgaactttaataagacagccgagcaggaatatggcgataaagaagtgaagctgcctcactggaccccaacactgcatacattccaggtgccacagaactacactaaagctaattgcacttattgtaacaccagggagtacacatttagttataaggggtgctgtttctactttactaagaaaaagcacacctggaatggatgcttccaggcctgtgctgaactgtatccatgcacatacttttatggcccaactcccgacatcctgcccgtggtgaccaggaacctgaatgccattgagtccctgtgggtgggagtgtacagggtgggagaaggcaactggacctccctggatggcgggacattcaaagtgtaccagatttttggctctcattgcacttatgtgtctaagttcagtaccgtgcccgtgtcacaccatgagtgtagctttctgaagccttgcctgtgtgtgtctcagagaagcaactcctga

SEQ ID NO: 130 - protein, 214 aa, synthetic construct
  REGION 1..214: note = Synthetic
MPMGSLQPLATLYLLGMLVASVLAGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 131 - DNA, 414 bp, Epstein Barr Virus
  source 1..414: mol_type = other DNA
atgcgggccgtgggggtgttcctggctatctgcctggtgactatttttgtgctgccaacctggggaaactgggcttacccttgctgtcacgtgacccagctcagggcccagcatctgctggctctggagaacatcagcgacatttatctggtgtccaatcagacatgcgatgggttcagcctggcctccctgaacagccccaagaacggatctaatcagctcgtgatctcccggtgtgctaacggcctgaatgtcgtgagtttctttatctcaattctgaaaaggagctcctctgctctgacaggacacctgagggagctgctgaccacactggaaactctgtacggaagtttctcagtggaagacctgtttggcgccaacctgaatcggtatgcttggcatagaggcgggtga

SEQ ID NO: 132 - protein, 137 aa, Epstein Barr Virus; identical to SEQ ID NO: 65

SEQ ID NO: 133 - DNA, 1458 bp, synthetic construct
  misc_feature 1..1458: note = Synthetic
  source 1..1458: mol_type = other DNA
atgcccatgggcagcctgcagcccctggccaccctgtacctgctgggcatgctggtggctagcatggagttcctgaagaggagcttcgcccctctgaccgagaagcagtggcaggagatcgacaacagggccagggagatcttcaagacccagctgtacggcaggaagttcgtggacgtggagggcccctacggctgggagtacgccgcccaccccctgggcgaggtggaggtgctgagcgacgagaacgaggtggtgaagtggggcctgaggaagagcctgcccctgatcgagctgagggccaccttcaccctggacctgtgggagctggacaacctggagaggggcaagcccaacgtggacctgagcagcctggaggagaccgtgaggaaggtggccgagttcgaggacgaggtgatcttcaggggctgcgagaagagcggcgtgaagggcctgctgagcttcgaggagaggaagatcgagtgcggcagcacccccaaggacctgctggaggccatcgtgagggccctgagcatcttcagcaaggacggcatcgagggcccctacaccctggtgatcaacaccgacaggtggatcaacttcctgaaggaggaggccggccactaccccctggagaagagggtggaggagtgcctgaggggcggcaagatcatcaccacccccaggatcgaggacgccctggtggtgagcgagaggggcggcgacttcaagctgatcctgggccaggacctgagcatcggctacgaggacagggagaaggacgccgtgaggctgttcatcaccgagaccttcaccttccaggtggtgaaccccgaggccctgatcctgctgaagtccggatctggcggcggtagcggcggtggcggaggaagagtggccgctgccgctatcacctgggtgccaaaacctaatgtggaagtgtggcctgtggacccaccacctccagtgaactttaataagacagccgagcaggaatatggcgataaagaagtgaagctgcctcactggaccccaacactgcatacattccaggtgccacagaactacactaaagctaattgcacttattgtaacaccagggagtacacatttagttataaggggtgctgtttctactttactaagaaaaagcacacctggaatggatgcttccaggcctgtgctgaactgtatccatgcacatacttttatggcccaactcccgacatcctgcccgtggtgaccaggaacctgaatgccattgagtccctgtgggtgggagtgtacagggtgggagaaggcaactggacctccctggatggcgggacattcaaagtgtaccagatttttggctctcattgcacttatgtgtctaagttcagtaccgtgcccgtgtcacaccatgagtgtagctttctgaagccttgcctgtgtgtgtctcagagaagcaactcctga

SEQ ID NO: 134 - protein, 485 aa, synthetic construct
  REGION 1..485: note = Synthetic
MPMGSLQPLATLYLLGMLVASMEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKSGSGGGSGGGGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 135 - DNA, 2040 bp, synthetic construct
  misc_feature 1..2040: note = Synthetic
  source 1..2040: mol_type = other DNA
atgcagctcctgtgcgtgttctgtctggtgctgctgtgggaagtgggagccgcttctctgagtgaggtgaagctgcacctggacattgaaggccacgcctcccattacactatcccttggaccgagctgatggctaaagtgccaggactgtctcctgaggctctgtggcgggaagctaatgtgaccgaggatctggcctctatgctgaacagatacaagctgatctataaaaccagtggcacactggggattgctctggctgagccagtggacatccccgccgtgtcagaaggaagcatgcaggtggatgctagtaaggtgcatccaggggtgattagcggactgaacagcccagcttgcatgctgagcgctcctctggagaaacagctcttctactatatcggcaccatgctgcctaatacacggccacacagctacgtgttttatcagctcagatgtcatctgtcctacgtggccctgtctattaacggggacaagttccagtatacaggagctatgacttccaaatttctgatgggaacttacaagcgggtgaccgagaaaggcgatgaacacgtgctgtctctggtgttcgggaagacaaaagacctgcccgatctgagaggacccttttcctacccttctctgactagtgcccagtcaggcgactatagcctggtgatcgtgaccacattcgtgcactacgctaacttccataattattttgtgcccaatctgaaggatatgttttcccgggccgtgaccatgacagccgcttcttacgctagatatgtgctgcagaagctggtgctgctggagatgaaaggcgggtgccgggagcctgaactggacactgaaaccctgactaccatgttcgaggtgtccgtggccttctttaaagtgggacacgctgtgggagagacaggaaacggatgcgtggacctgagatggctggccaagagcttctttgaactgaccgtgctgaaagatatcattggaatctgttacggcgccacagtgaaaggaatgcagagctatggcctggagaggctggccgctatgctgatggccaccgtgaagatggaggaactgggccacctgacaactgagaaacaggaatacgctctgaggctggctaccgtgggatacccaaaggccggggtgtattccggactgattggaggcgccacatctgtgctgctgagtgcttataataggcacccactgttccagcccctgcatacagtgatgcgcgagactctgtttatcgggtctcatgtggtgctgcgggaactgagactgaatgtgaccacacagggacccaacctggccctgtaccagctcctgagtactgccctgtgctcagctctggagattggagaagtgctgaggggactggccctggggaccgagtcaggactgttcagcccttgttatctgtcactgaggtttgacctgactcgcgataagctgctgagcatggccccacaggaagctaccctggaccaggccgctgtgagcaatgccgtggatggattcctgggcaggctgtccctggagagggaagaccgcgatgcctggcacctgccagcttacaagtgcgtggaccgcctggataaagtgctgatgatcattcccctgatcaacgtgaccttcatcattagctccgacagggaagtgagaggcagcgctctgtacgaagcttccactacctatctgtctagttcactgtttctgtcacctgtgattatgaataagtgtagccagggagctgtggctggagagcccagacagatcccaaagattcagaacttcacacgcactcagaaaagttgcatcttctgtggctttgccctgctgtcatacgatgagaaagaagggctggagacaactacctatattacatctcaggaagtgcagaacagtatcctgagctccaattacttcgactttgataacctgcacgtgcattatctgctgctgacaactaacggcaccgtgatggagatcgctggactgtacgaggaaagggctcactga

SEQ ID NO: 136 - protein, 679 aa, synthetic construct
  REGION 1..679: note = Synthetic
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAH

SEQ ID NO: 137 - DNA, 84 bp, Picornavirus
  source 1..84: mol_type = other DNA
agcgggtccggagctccagtgaaacagaccctgaactttgacctgctgaagctggcaggggatgtggagagcaatcctggccca

SEQ ID NO: 138 - protein, 28 aa, Picornavirus
SGSGAPVKQTLNFDLLKLAGDVESNPGP

SEQ ID NO: 139 - DNA, 72 bp, Homo sapiens
  source 1..72: mol_type = other DNA
atgccaatgggcagcctgcagccactggcaactctgtacctgctgggaatgctggtggcatccgtcctggca

SEQ ID NO: 140 - protein, 24 aa, Homo sapiens
MPMGSLQPLATLYLLGMLVASVLA

SEQ ID NO: 141 - DNA, 3087 bp, synthetic construct
  misc_feature 1..3087: note = NAM encoding ferritin-gH-F2A-gL
  source 1..3087: mol_type = other DNA
atgcagctgctgtgtgtgttctgtctggtcctgctgtgggaagtcggagccgcctctctgagcgaagtgaaactgcacctggatattgaagggcacgcctctcattacaccatcccttggacagagctgatggctaaggtgcctggactgagtccagaagctctgtggcgcgaggcaaacgtgaccgaagacctggcctccatgctgaatcgatacaaactgatctacaagacatctgggactctgggaatcgccctggctgaacccgtggacattcctgctgtcagtgagggctcaatgcaggtggatgcaagcaaagtgcacccaggagtcatctctggcctgaacagtcccgcctgcatgctgtccgctcctctggagaagcagctgttctactatattgggacaatgctgccaaacactagaccccacagctacgtgttttatcagctgaggtgtcatctgagctacgtcgcactgtccatcaatggagacaaattccagtatactggcgccatgacctctaagtttctgatgggaacctacaaaagggtgacagaaaagggcgatgagcacgtgctgagcctggtcttcggcaaaaccaaggacctgcccgatctgcgcgggccttttagctacccatccctgacatctgcccagagtggcgactatagcctggtcatcgtcaccacattcgtgcactacgctaacttccataattattttgtccctaacctgaaggatatgttttccagggcagtgactatgaccgccgcttcttacgcccgctatgtgctgcagaaactggtcctgctggagatgaagggaggatgccgagaaccagagctggacacagaaactctgactaccatgttcgaggtgtccgtcgctttctttaaagtggggcacgcagtcggagaaacaggcaatgggtgcgtggacctgagatggctggccaaaagcttctttgagctgaccgtgctgaaggatatcattggcatctgttacggggctacagtcaagggcatgcagtcctatgggctggagcggctggcagccatgctgatggccaccgtgaaaatggaggaactgggacacctgacaactgaaaagcaggagtacgccctgagactggctactgtgggctacccaaaggccggagtctatagcggactgatcggaggagcaacctcagtgctgctgagcgcttataaccgacaccccctgttccagcctctgcatactgtgatgcgggaaaccctgtttattggctcccatgtggtcctgcgagagctgcggctgaacgtgaccacacaggggcccaatctggctctgtaccagctgctgtctacagcactgtgcagtgccctggaaatcggagaggtgctgaggggactggcactgggaactgaatccggactgttctctccctgttatctgagtctgaggtttgacctgactcgcgataagctgctgtcaatggctcctcaggaggcaaccctggaccaggctgcagtgtcaaacgcagtcgatggcttcctgggacgactgagcctggaaagagaggacagggatgcatggcacctgcctgcctacaaatgcgtggacagactggataaggtcctgatgatcattccactgatcaatgtgaccttcatcattagctccgaccgagaagtccgaggctccgcactgtacgaggcttctactacctatctgtctagttcactgtttctgtcacccgtgatcatgaacaaatgtagccagggagcagtcgcaggagagccacgacagatccccaaaattcagaatttcacccgaacacagaagtcttgcattttctgtggatttgccctgctgagttacgatgaaaaggagggcctggaaacaactacctatatcacaagtcaggaggtgcagaattcaattctgagctccaactacttcgactttgataatctgcacgtgcattatctgctgctgacaactaacgggaccgtcatggaaatcgcaggactgtacgaggaaagagcacactcaggaggaagcggagagtcccaggtgaggcagcagttctctaaagacattgagaagctgctgaacgaacaagtgaataaggagatgcagtctagtaacctgtacatgagtatgtcaagctggtgctatacccactcactggacggagcaggcctgttcctgtttgatcacgccgctgaggaatacgaacatgccaagaaactgatcatttttctgaacgagaacaacgtgcctgtccagctgacatcaatcagcgctccagaacataaattcgagggcctgactcagatctttcagaaggcatacgaacacgagcagcatatttccgaatctatcaacaatattgtggaccacgccatcaagagcaaggatcatgcaaccttcaattttctgcagtggtacgtggccgagcagcacgaggaagaggtcctgttcaaagacatcctggataagatcgaactgattggaaacgagaatcatggcctgtacctggccgatcagtatgtgaaaggcattgctaaatctcgaaagagtgggtcacggaagcgaagaagcgggtccggagctccagtgaaacagaccctgaactttgacctgctgaagctggcaggggatgtggagagcaatcctggcccaatgagggccgtgggggtcttcctggctatctgtctggtgaccatttttgtcctgccaacatggggaaactgggcctacccatgctgtcacgtgacccagctgcgagctcagcatctgctggcactggagaacatcagcgacatctacctggtgagcaatcagacatgcgatgggttctctctggccagtctgaattcacctaaaaacggatctaatcagctggtcatcagtaggtgtgctaacggcctgaatgtggtcagtttctttatctcaattctgaagcggtcctctagtgccctgacaggccacctgagagaactgctgaccacactggagactctgtacgggtctttcagtgtggaggacctgtttggagcaaacctgaatcgctatgcatggcatcgaggaggatga

SEQ ID NO: 142 - protein, 1028 aa, synthetic construct
  REGION 1..1028: note = Protein encoded by SEQ ID NO:141 (ferritin-gH-F2A-gL)
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHSGGSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGSRKRRSGSGAPVKQTLNFDLLKLAGDVESNPGPMRAVGVFLAICLVTIFVLPTWGNWAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGG

SEQ ID NO: 143 - DNA, 3825 bp, synthetic construct
  misc_feature 1..3825: note = NAM encoding ferritin-gH-F2A-gL-F2A-gp42
  source 1..3825: mol_type = other DNA
atgcagctgctgtgtgtgttctgtctggtcctgctgtgggaagtcggagccgcctctctgagcgaagtgaaactgcacctggatattgaagggcacgcctctcattacaccatcccttggacagagctgatggctaaggtgcctggactgagtccagaagctctgtggcgcgaggcaaacgtgaccgaagacctggcctccatgctgaatcgatacaaactgatctacaagacatctgggactctgggaatcgccctggctgaacccgtggacattcctgctgtcagtgagggctcaatgcaggtggatgcaagcaaagtgcacccaggagtcatctctggcctgaacagtcccgcctgcatgctgtccgctcctctggagaagcagctgttctactatattgggacaatgctgccaaacactagaccccacagctacgtgttttatcagctgaggtgtcatctgagctacgtcgcactgtccatcaatggagacaaattccagtatactggcgccatgacctctaagtttctgatgggaacctacaaaagggtgacagaaaagggcgatgagcacgtgctgagcctggtcttcggcaaaaccaaggacctgcccgatctgcgcgggccttttagctacccatccctgacatctgcccagagtggcgactatagcctggtcatcgtcaccacattcgtgcactacgctaacttccataattattttgtccctaacctgaaggatatgttttccagggcagtgactatgaccgccgcttcttacgcccgctatgtgctgcagaaactggtcctgctggagatgaagggaggatgccgagaaccagagctggacacagaaactctgactaccatgttcgaggtgtccgtcgctttctttaaagtggggcacgcagtcggagaaacaggcaatgggtgcgtggacctgagatggctggccaaaagcttctttgagctgaccgtgctgaaggatatcattggcatctgttacggggctacagtcaagggcatgcagtcctatgggctggagcggctggcagccatgctgatggccaccgtgaaaatggaggaactgggacacctgacaactgaaaagcaggagtacgccctgagactggctactgtgggctacccaaaggccggagtctatagcggactgatcggaggagcaacctcagtgctgctgagcgcttataaccgacaccccctgttccagcctctgcatactgtgatgcgggaaaccctgtttattggctcccatgtggtcctgcgagagctgcggctgaacgtgaccacacaggggcccaatctggctctgtaccagctgctgtctacagcactgtgcagtgccctggaaatcggagaggtgctgaggggactggcactgggaactgaatccggactgttctctccctgttatctgagtctgaggtttgacctgactcgcgataagctgctgtcaatggctcctcaggaggcaaccctggaccaggctgcagtgtcaaacgcagtcgatggcttcctgggacgactgagcctggaaagagaggacagggatgcatggcacctgcctgcctacaaatgcgtggacagactggataaggtcctgatgatcattccactgatcaatgtgaccttcatcattagctccgaccgagaagtccgaggctccgcactgtacgaggcttctactacctatctgtctagttcactgtttctgtcacccgtgatcatgaacaaatgtagccagggagcagtcgcaggagagccacgacagatccccaaaattcagaatttcacccgaacacagaagtcttgcattttctgtggatttgccctgctgagttacgatgaaaaggagggcctggaaacaactacctatatcacaagtcaggaggtgcagaattcaattctgagctccaactacttcgactttgataatctgcacgtgcattatctgctgctgacaactaacgggaccgtcatggaaatcgcaggactgtacgaggaaagagcacactcaggaggaagcggagagtcccaggtgaggcagcagttctctaaagacattgagaagctgctgaacgaacaagtgaataaggagatgcagtctagtaacctgtacatgagtatgtcaagctggtgctatacccactcactggacggagcaggcctgttcctgtttgatcacgccgctgaggaatacgaacatgccaagaaactgatcatttttctgaacgagaacaacgtgcctgtccagctgacatcaatcagcgctccagaacataaattcgagggcctgactcagatctttcagaaggcatacgaacacgagcagcatatttccgaatctatcaacaatattgtggaccacgccatcaagagcaaggatcatgcaaccttcaattttctgcagtggtacgtggccgagcagcacgaggaagaggtcctgttcaaagacatcctggataagatcgaactgattggaaacgagaatcatggcctgtacctggccgatcagtatgtgaaaggcattgctaaatctcgaaagagtgggtcacggaagcgaagaagcgggtccggagctccagtgaaacagaccctgaactttgacctgctgaagctggcaggggatgtggagagcaatcctggcccaatgagggccgtgggggtcttcctggctatctgtctggtgaccatttttgtcctgccaacatggggaaactgggcctacccatgctgtcacgtgacccagctgcgagctcagcatctgctggcactggagaacatcagcgacatctacctggtgagcaatcagacatgcgatgggttctctctggccagtctgaattcacctaaaaacggatctaatcagctggtcatcagtaggtgtgctaacggcctgaatgtggtcagtttctttatctcaattctgaagcggtcctctagtgccctgacaggccacctgagagaactgctgaccacactggagactctgtacgggtctttcagtgtggaggacctgtttggagcaaacctgaatcgctatgcatggcatcgaggaggaagaaagaggcgatcaggcagcggagcacctgtcaaacagaccctgaacttcgacctgctgaagctggctggagatgtggagagcaatcccgggcctatgccaatgggcagcctgcagccactggcaactctgtacctgctgggaatgctggtggcatccgtcctggcaggaggacgagtggcagcagctgcaatcacatgggtccccaaacctaacgtggaagtctggccagtggacccccctccaccagtcaactttaataagaccgccgaacaggagtatggcgataaagaggtgaagctgcctcactggactccaaccctgcatactttccaggtgcctcagaactacaccaaagccaattgcacatattgtaacactagagagtacaccttttcttataaggggtgctgtttctactttacaaagaaaaagcacacttggaacggatgcttccaggcttgtgcagagctgtatccatgcacttacttttatggaccaaccccagacatcctgccagtggtcaccaggaacctgaatgccattgaaagcctgtgggtgggagtctaccgagtgggagagggcaattggacaagcctggatgggggaactttcaaagtgtaccagatctttggctcccattgcacctatgtcagcaagttctccacagtgcccgtctcacaccatgagtgtagctttctgaagccttgcctgtgtgtgagccagcggtccaactcttga

SEQ ID NO: 144 - protein, 1274 aa, synthetic construct
  REGION 1..1274: note = Protein encoded by SEQ ID NO:143 (ferritin-gH-F2A-gL-F2A-gp42)
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHSGGSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGSRKRRSGSGAPVKQTLNFDLLKLAGDVESNPGPMRAVGVFLAICLVTIFVLPTWGNWAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGGRKRRSGSGAPVKQTLNFDLLKLAGDVESNPGPMPMGSLQPLATLYLLGMLVASVLAGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 145 - DNA, 2595 bp, synthetic construct
  misc_feature 1..2595: note = gH-(SGGG)2-ferritin sequence
  source 1..2595: mol_type = other DNA
atgcagctcctgtgcgtgttctgtctggtgctgctgtgggaagtgggagccgcttctctgagtgaggtgaagctgcacctggacattgaaggccacgcctcccattacactatcccttggaccgagctgatggctaaagtgccaggactgtctcctgaggctctgtggcgggaagctaatgtgaccgaggatctggcctctatgctgaacagatacaagctgatctataaaaccagtggcacactggggattgctctggctgagccagtggacatccccgccgtgtcagaaggaagcatgcaggtggatgctagtaaggtgcatccaggggtgattagcggactgaacagcccagcttgcatgctgagcgctcctctggagaaacagctcttctactatatcggcaccatgctgcctaatacacggccacacagctacgtgttttatcagctcagatgtcatctgtcctacgtggccctgtctattaacggggacaagttccagtatacaggagctatgacttccaaatttctgatgggaacttacaagcgggtgaccgagaaaggcgatgaacacgtgctgtctctggtgttcgggaagacaaaagacctgcccgatctgagaggacccttttcctacccttctctgactagtgcccagtcaggcgactatagcctggtgatcgtgaccacattcgtgcactacgctaacttccataattattttgtgcccaatctgaaggatatgttttcccgggccgtgaccatgacagccgcttcttacgctagatatgtgctgcagaagctggtgctgctggagatgaaaggcgggtgccgggagcctgaactggacactgaaaccctgactaccatgttcgaggtgtccgtggccttctttaaagtgggacacgctgtgggagagacaggaaacggatgcgtggacctgagatggctggccaagagcttctttgaactgaccgtgctgaaagatatcattggaatctgttacggcgccacagtgaaaggaatgcagagctatggcctggagaggctggccgctatgctgatggccaccgtgaagatggaggaactgggccacctgacaactgagaaacaggaatacgctctgaggctggctaccgtgggatacccaaaggccggggtgtattccggactgattggaggcgccacatctgtgctgctgagtgcttataataggcacccactgttccagcccctgcatacagtgatgcgcgagactctgtttatcgggtctcatgtggtgctgcgggaactgagactgaatgtgaccacacagggacccaacctggccctgtaccagctcctgagtactgccctgtgctcagctctggagattggagaagtgctgaggggactggccctggggaccgagtcaggactgttcagcccttgttatctgtcactgaggtttgacctgactcgcgataagctgctgagcatggccccacaggaagctaccctggaccaggccgctgtgagcaatgccgtggatggattcctgggcaggctgtccctggagagggaagaccgcgatgcctggcacctgccagcttacaagtgcgtggaccgcctggataaagtgctgatgatcattcccctgatcaacgtgaccttcatcattagctccgacagggaagtgagaggcagcgctctgtacgaagcttccactacctatctgtctagttcactgtttctgtcacctgtgattatgaataagtgtagccagggagctgtggctggagagcccagacagatcccaaagattcagaacttcacacgcactcagaaaagttgcatcttctgtggctttgccctgctgtcatacgatgagaaagaagggctggagacaactacctatattacatctcaggaagtgcagaacagtatcctgagctccaattacttcgactttgataacctgcacgtgcattatctgctgctgacaactaacggcaccgtgatggagatcgctggactgtacgaggaaagggctcactctggcggcggtagcggcggtggctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagagcggatcctag

SEQ ID NO: 146 - protein, 864 aa, synthetic construct
  REGION 1..864: note = Amino acid sequence encoded by SEQ ID NO:146 (gH-(SGGG)2-ferritin sequence)
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHSGGGSGGGSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS